(19) United States
(12) Patent Application Publication
(10) Pub. No.: US 2025/0059537 A1
(43) Pub. Date: Feb. 20, 2025

(54) THERAPEUTIC OLIGONUCLEOTIDES HAVING AN INTER-NUCLEOSIDE AMIDE LINKAGE

(71) Applicant: OXFORD UNIVERSITY INNOVATION LIMITED, Oxford (GB)

(72) Inventors: Tom BROWN, Oxford (Oxfordshire) (GB); Afaf Helmy EL-SAGHEER, Oxford (Oxfordshire) (GB); Ysobel Ruth BAKER, Oxford (Oxfordshire) (GB); Pawan KUMAR, Oxford (Oxfordshire) (GB)

(21) Appl. No.: 18/719,979

(22) PCT Filed: Dec. 16, 2022
(86) PCT No.: PCT/GB2022/053283

§ 371 Date: Jun. 14, 2024

(30) Foreign Application Priority Data
Dec. 16, 2021 (GB) 2118332.2

(51) Int. Cl. C12N 15/113

(52) U.S. Cl. CPC C12N 15/113; C12N 2310/11; C12N 2310/315; C12N 2310/321; C12N 2310/3231

(57) ABSTRACT

The present invention relates to a therapeutic oligonucleotide having a 5′ and a 3′ end and comprising a sequence of nucleosides linked together by inter-nucleoside linkages, wherein: at least one inter-nucleoside linkage is an amide linker moiety; at least one inter-nucleoside linkage is a phosphorothioate linker moiety; and at least one nucleoside present in the oligonucleotide is a locked nucleoside; wherein the at least one locked nucleoside is directly attached to the 3′ end or the 5′ end of the amide linker moeity. The oligonucleotide has preferably structure (IIa) or (llb):

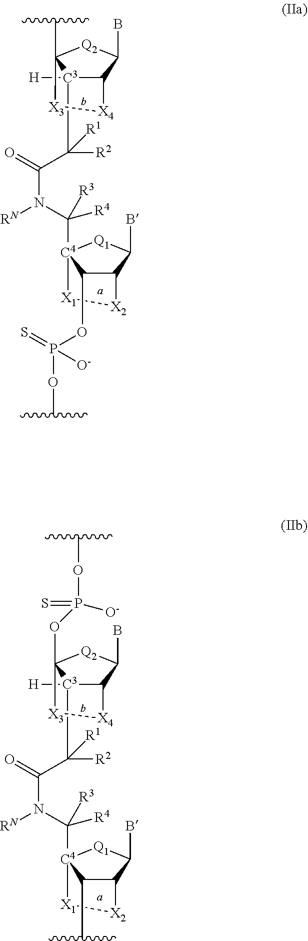

The project leading to this application has received funding from the European Union's Horizon 2020 research and innovation programme under the Marie Sklodowska-Curie grant agreement No 656872.

### INTRODUCTION

The present invention relates to oligonucleotides.

### BACKGROUND OF THE INVENTION

Oligonucleotides (ONs) are fundamental to many areas of molecular biology and are essential tools in technologies such as DNA sequencing, forensic and genetic analysis. They can also be used therapeutically.

Although the concept of binding RNA to correct disease was first demonstrated in 1978, antisense oligonucleotides (ASOs) are only just starting to deliver on their initial promise. To date, nine ASOs have now been approved for clinical use indicating their increasing therapeutic potential. All of these, however, are for rare, severe life limiting diseases.

The field recently received a boost by the approval by the European regulators of the oligonucleotide drug Inclisiran for lowering LDL cholesterol levels in patients with hypercholesterolemia. Unlike previous oligonucleotide therapeutics, Inclisiran is used to treat a common disease. However, although a major advance, and an insight into what the field can offer, Inclisiran is a special case of delivery to the liver. For other organs, inefficient biodistribution, poor cellular delivery, and toxicity prevent the wider adoption of this technology.

To address these issues new oligonucleotide analogues are greatly sought after. There are RNA targets for many more diseases than there are conventional protein targets, including incurable cancers, genetic disorders, and debilitating infectious diseases, hence improvements in ASO chemistry is likely to have huge societal benefit.

Therapeutic oligonucleotides are short single-stranded analogues of DNA that bind to RNA to regulate gene expression and alter protein synthesis^{1, 2}. They act as steric blockers of translation³, recruit RNase-H leading to degradation of mRNA⁴, or modulate pre-mRNA splicing⁵⁻⁷. They can also be formulated as double-stranded siRNA constructs for gene silencing⁸. Oligonucleotides have attracted much attention as therapeutic agents due to their logical design criteria based on Watson Crick base pairing, high target specificity, and extensive range of potential disease targets. Indeed, there are far more potential RNA targets than conventional protein targets for human diseases such as cancers, genetic disorders, and debilitating infectious diseases, many of which are undruggable using existing approaches⁹. Hence improvements in ASO chemistry are likely to have huge societal benefit.

Therapeutic oligonucleotides hold great promise against currently untreatable diseases, but are hampered by poor cellular uptake and limited bioavailability.

To achieve a therapeutic response an oligonucleotide must be stable in vivo, bind to its target RNA with high selectivity and affinity, and display good pharmacokinetic properties². Unmodified oligonucleotides are rapidly digested by nucleases in cells and are therefore unsuitable for use as drugs. This limitation has led to a plethora of modifications aiming to provide nuclease resistance whilst retaining high target affinity¹⁰. The most successful constructs combine 2′-sugar substitutions, including 2′-OMe (2′OMe), 2′-O-(2-methoxyethyl) and 2′-fluoro,¹¹⁻¹³ with phosphorothioate (PS) backbones^{12, 14} The PS group is essential; it provides resistance to in vivo degradation and improves cell uptake. However, it also reduces RNA target affinity, which is overcome by the additional 2′-sugar modifications². Whilst these developments have led to the FDA and EMA approval of several oligonucleotide therapies¹⁵, limited efficacy, off-target effects and toxicity issues inhibit the wider adoption of oligonucleotides in the clinic¹⁶. Improvements in these areas would be transformative.

Poor uptake into cells remains a major obstacle; generally less than 1% of an administered oligonucleotide typically reaches its target¹⁷. Reducing the net anionic charge of the oligonucleotide by full or partial replacement of the phosphodiester backbone with neutral linkages is a potential means of increasing cell permeability and nuclease resistance^{10, 18} However, most of the current modifications suffer from reduced duplex stability when hybridised to RNA¹⁰. In contrast, peptide nucleic acid (PNA),¹⁹ which is uncharged, has high target affinity, but poor aqueous solubility and inefficient cell penetration. This makes it therapeutically unsuitable,²¹ although studies to address this issue are ongoing²².

Oligonucleotides containing the artificial amide backbone AM1 have shown initial promise²³⁻²⁵ (FIG. 1), having potential applications in the siRNA field^{26,27}. They form more stable duplexes with complementary RNA (A-form) than DNA (B-form).²³ However, synthesis of the required carboxylic acid monomers has not been established for all four canonical nucleobase analogues²⁹, limiting the potential of this modification.

Locked nucleic acid oligonucleotides (LNA-ONs) are well established³²; binding to complementary RNA targets with very high affinity^{33, 34, 35}. However, LNA-ONs have not yet been clinically approved, largely due to challenges with toxicity.³² The extreme duplex stabilising effects of LNA can result in binding to imperfectly matched RNA strands, causing undesirable off-target effects. Nevertheless, LNA is a powerful modification for enhancing target affinity, and combining it with artificial DNA backbones is an exciting prospect. In this context LNA has been mixed with charge-neutral backbones including various triazoles³⁶⁻³⁸, carbamates³⁹, and amides^{40, 41}. In all cases where duplex stability was determined, stabilisation with RNA targets only occurred when the LNA was positioned on the 3′-side of the modified backbone where it directly influences the attached phosphodiester backbone. The LNA sugar has consistently failed to stabilise artificial linkages when located on their 5′-side.

The present invention was devised with the foregoing in mind.

### SUMMARY OF THE INVENTION

According to one aspect of the present invention, there is provided an oligonucleotide as defined herein, or a pharmaceutically acceptable salt or solvate thereof.

According to a second aspect of the present invention, there is provided a process for making an oligonucleotide as defined herein, or a pharmaceutically acceptable salt or solvate thereof.

According to a third aspect of the present invention, there is provided an oligonucleotide as defined herein, or a pharmaceutically acceptable salt or solvate thereof, for use in therapy.

According to a fourth aspect of the present invention, there is provided a use of an oligonucleotide as defined herein, or a pharmaceutically acceptable salt or solvate thereof, as antisense RNA or interference RNA (RNAi, e.g. siRNA or miRNA) or an RNA component of a CRISPR-Cas system (e.g. crRNA, tracrRNA or gRNA).

According to another aspect of the present invention, there is provided a method for amplifying an oligonucleotide sequence as defined herein.

According to another aspect of the present invention, there is provided a method for replicating an oligonucleotide sequence as defined herein.

According to another aspect of the present invention, there is provided a method for producing a ribonucleic acid (RNA) sequence or deoxyribonucleic acid (DNA) sequence as defined herein.

### DETAILED DESCRIPTION OF THE INVENTION

### Definitions

Unless otherwise stated, the following terms used in the specification and claims have the following meanings set out below.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or examples of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” includes both straight and branched chain alkyl groups. References to individual alkyl groups such as “propyl” are specific for the straight chain version only and references to individual branched chain alkyl groups such as “isopropyl” are specific for the branched chain version only. For example, “(1-6C)alkyl” includes (1-4C)alkyl, (1-3C)alkyl, propyl, isopropyl and t-butyl. A similar convention applies to other radicals, for example “phenyl(1-6C)alkyl” includes phenyl(1-4C)alkyl, benzyl, 1-phenylethyl and 2-phenylethyl.

The term “(m-nC)” or “(m-nC) group” used alone or as a prefix, refers to any group having m to n carbon atoms.

The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

The phrase “oligonucleotide of the invention” means those oligonucleotides which are disclosed herein, both generically and specifically, or pharmaceutically acceptable salts or solvates thereof.

The term “oligonucleotide” refers to a polynucleotide strand. It will be appreciated by those skilled in the art that an oligonucleotide has a 5′ and a 3′ end and comprises a sequence of nucleosides linked together by inter-nucleoside linkages.

The terms “oligonucleotide analogue” and “nucleotide analogue” refer to any modified synthetic analogues of oligonucleotides or nucleotides respectively that are known in the art. Examples of oligonucleotide analogues include peptide nucleic acids (PNAs), morpholino oligonucleotides, phosphorothioate oligonucleotides, phosphorodithioate oligonucleotides, alkylphosphonate oligonucleotides, acylphosphonate oligonucleotides and phosphoramidate oligonucleotides.

“Nucleobase” refers to a substituted or unsubstituted nitrogen-containing parent heteroaromatic ring of a type that is commonly found in nucleic acids. typically, but not necessarily, the nucleobase is capable of forming Watson-Crick and/or Hoogsteen hydrogen bonds with an appropriately complementary nucleobase. The nucleobases may be naturally occurring, such as the naturally-occurring encoding nucleobases A, G, C, T and U, or they may be modified or synthetic. The term “nucleobase” as defined herein therefore refers to both naturally occurring nucleobases which function as the fundamental units of genetic code (i.e. adenine (A), cytosine (C), guanine (G), thymine (T), and uracil (U)) and also any modified or synthetic nucleobases which are known in the art. The skilled person will appreciate there to be numerous natural and synthetic nucleobase analogues available in the art which could be employed in the present invention. As such, the skilled person will readily be able to identify suitable nucleobase analogues for use in the present invention. Commonly available nucleobase analogues are commercially available from a number of sources (for example, see the Glen Research catalogue).

It will also be appreciated that the term “modified nucleobases” covers but is not limited to universal/degenerate bases (e.g. 3-nitropyrrole, 5-nitroindole and hypoxanthine); fluorescent bases (e.g. tricyclic cytosine analogues (tCO, tCS) and 2-aminopurine); base analogues bearing reactive groups selected from alkynes, thiols or amines; and base analogues that can crosslink oligonucleotides to DNA, RNA or proteins (e.g. 5-bromouracil or 3-cyanovinyl carbazole).

Common modified or synthetic nucleobases include 2-aminoadenine, 5-propynylcytosine, 5-propynyluracil, 5-methylcytosine, 3-methyluracil, 5,6-dihydrouracil, 4-thiouracil, 5-bromouracil, 5-chlorouracil, 5-iodouracil, 6-dimethyl aminopurine, 6-methyl amino purine, 2-amino purine, 2,6-diamino purine, 6-amino-8-bromo purine, inosine, 5-methyl cytosine, 7-deazaadenine, 7-deazaguanosine, 3-cyanovinyl carbazole, 3-nitropyrrole, 5-nitroindole, hypoxanthine and G-clamp (a tricyclic aminoethyl-phenoxazine 2′-deoxyCytidine analogue which has the structure

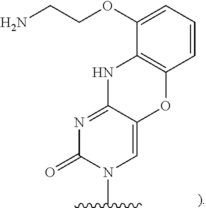 [SMILES: *n1cc2c(nc1=O)Nc1c(OCCN)cccc1O2]

Additional non-limiting examples of modified or synthetic nucleobases of which the target nucleic acid may be composed can be found in Fasman, CRC PRACTICAL HANDBOOK OF BIOCHEMISTRY AND McOLECULAR BIOLOGY, 1985, pp. 385-392; Beilstein's Handbuch der Organischen Chemie, Springer Verlag, Berlin and Chemical Abstracts, the entirety of which is incorporated herein by reference, and which provide references to publications describing the structures, properties and preparation of such nucleobases.

As will be recognized by those of skill in the art, many of the above-described modified or synthetic nucleobases are capable of forming Watson-Crick base pairing interactions with the naturally occurring encoding nucleobases A, T, C, G and U. However, in certain embodiments of the invention, it may be desirable to include in a nucleobase polymer synthetic nucleobases which are not capable of forming Watson-Crick base pairs with either the naturally occurring encoding nucleobases A, T, C, G, and U and/or common analogs thereof, but that are capable of forming non-standard (i.e., non-Watson-Crick) base pairs with one another. Nucleobases having these properties are referred to herein as “non-standard synthetic” nucleobases. Examples of such non-standard synthetic nucleobases include, but are not limited to, iso-guanine (iso-G), iso-cytosine (iso-C), xanthine (X), kappa (K), nucleobase H, nucleobase J, nucleobase M and nucleobase N (see U.S. Pat. No. 6,001,983). These non-standard synthetic nucleobases base-pair with one another to form the following non-standard base pairs: iso-C·iso-G, K·X, H·J and M·N. Each of these non-standard base pairs has three hydrogen bonds. Additional non-standard synthetic nucleobases, as well as methods for their synthesis and methods for incorporating them into nucleobase polymers are found in U.S. Pat. Nos. 5,432,272, 5,965,364 and 6,001,983, the disclosures of which are incorporated herein by reference.

The nucleobase is attached to a sugar moiety (typically ribose or deoxyribose) or a ribose or deoxyribose mimic, for example a chemically modified sugar derivative (e.g. a chemically modified ribose or deoxyribose) or a cyclic group that functions as a synthetic mimic of a ribose or deoxyribose sugar moiety (e.g. the morpholino ring present in morpholino oligonucleotides).

A chemically modified sugar derivative includes sugars modified at the 2′ position, for example to include 2′-O-methyl, 2′-O-methoxy-ethyl, 2′-NH₂ and 2′-F modifications. Such sugars may be located in any of the nucleotides present in the oligonucleotides of the present invention.

The term “nucleoside” is used herein to refer to a moiety composed of a sugar/a ribose or deoxyribose mimic bound to a nucleobase/nucleobase analogue. The term nucleoside as used herein excludes the inter-nucleoside linkage that connects adjacent nucleosides together. An “inter-nucleoside linkage” is a linking group that connects the rings of the sugar/ribose or deoxyribose mimic of adjacent nucleosides. Thus, a “nucleotide” is a nucleoside with one or more inter-nucleoside linkage attached.

The terms “locked nucleic acid”, “LNA” or “locked nucleoside” are used herein to refer to nucleic acids or nucleosides comprising a ribose or deoxyribose moiety in which the conformation of the ribose or deoxyribose ring is fixed or locked in a specific conformation, typically by a bridging group. Typically the bridging group connects the 2′ and 4′ carbon atoms of the ribose or deoxyribose rings and locks the ribose or deoxyribose in the 3′-endo conformation (which is often found in A-form duplexes). Examples of locked nucleic acid/nucleoside structures are well known in the art and are commercially available.

A suitable pharmaceutically acceptable salt of an oligonucleotide of the invention is, for example, an acid-addition salt of an oligonucleotide of the invention which is sufficiently basic, for example, an acid-addition salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of an oligonucleotide of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

It is also to be understood that certain oligonucleotides of the invention may exist in solvated as well as unsolvated forms such as, for example, hydrated forms.

### Oligonucleotides of the Invention

According to one aspect of the present invention, there is provided an oligonucleotide having a 5′ and a 3′ end and comprising a sequence of nucleosides linked together by inter-nucleoside linkages, wherein:
at least one inter-nucleoside linkage is an amide linker moiety;
at least one inter-nucleoside linkage is a phosphorothioate linker moiety; and
at least one nucleoside present in the oligonucleotide is a locked nucleoside;
wherein the at least one locked nucleoside is directly attached to the 3′ end or the 5′ end of the amide linker moeity;
   or a pharmaceutically acceptable salt or solvate thereof.

It will be appreciated by those skilled in the art that an inter-nucleoside linkage will have a 5′ end (or 5′ side) that links to the nucleoside on the 5′ side, and 3′ end (or 3′ side) that links to the nucleoside on the 3′ side of linkage. The 3′ and 5′ nomenclature is well established in the nucleic acid field.

The inventors have surprisingly found that the provision of an amide linker moiety, a phosphorothioate linker moiety and a locked nucleoside is associated with increased cell uptake of the modified oligonucleotide and also associated with reduced toxicity.

In addition, the oligonucleotides of the present invention are much more stable to nuclease degradation when compared to corresponding oligonucleotides comprising just locked nucleosides alone. This indicates that the oligonucleotides of the present invention will be suitable for use in vivo.

The combination of the two aforementioned advantages (namely the increased nuclease stability together with the increase in the thermal melting temperatures observed upon binding of the oligonucleotides of the present invention to complimentary DNA or RNA stands) makes the oligonucleotides of the present invention particularly advantageous.

In an embodiment, the at least one locked nucleoside is either directly attached to the 3′ end of the amide linker moiety. Suitably, the amide linker is attached to the 4′ carbon of the locked ribose or deoxyribose ring of the locked nucleoside.

The oligonucleotide may comprise multiple locked nucleosides in its sequence, for example there may be two, three, four, five or more locked nucleosides present. The additional locked nucleosides may be present at any position in the oligonucleotide.

In an embodiment, a the at least one locked nucleoside is directly attached to the 5′ end of the amide linker moiety. Suitably, the amide linker is attached to the 3′ carbon atom of the ribose or deoxyribose ring of the locked nucleoside.

In a particular embodiment, the oligonucleotide comprises at least two locked nucleosides, one of which is directly attached to the 3′ end of the amide linker moiety and the other of which is directly attached to the 5′ end of the amide linker moiety. This particular embodiment of the invention is expected to result in the oligonucleotide binding to complementary RNA tighter than in embodiments in which there is only one locked nucleoside or in comparison to an oligonucleotide comprising no locked nucleosides.

### The Amide Linker Moiety

Amide linkages known in the art are present in the oligonucleotides of the present invention. The amide linker moeity is an inter-nucleoside linkage that acts as a charge neutral mimic of the phosphodiester linkages found in naturally occurring polynucleotides.

Amide linkages suitably have the structure shown below:

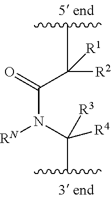

wherein:
R¹ and R² are each independently selected from hydrogen, (1-2C)alkyl, hydroxy, amino or halo;
R³ and R⁴ are each independently selected from hydrogen, (1-2C)alkyl, hydroxy, amino or halo; and
R^{N} is selected from hydrogen or (1-2C)alkyl.

Suitably, R¹ and R² are each independently selected from hydrogen or methyl.

Suitably, R³ and R⁴ are each independently selected from hydrogen or methyl.

Suitably, R^{N} is selected from hydrogen or methyl.

Suitably, each of R¹, R², R³, R⁴ and R^{N} is hydrogen.

### The Phosphorothioate Moiety

Phosphorothioate linkages known in the art are present in the oligonucleotides of the present invention. The phosphorothioate linker moeity is an inter-nucleoside linkage that acts as a mimic of the phosphodiester linkages found in naturally occurring polynucleotides.

The at least one phosphorothioate linkage may be located at any suitable position throughout the oligonucleotide. For example, the at least one phosphorothioate linker may be located at one or more of the following positions:
a. directly attached to the 3′ end of the dinucleotide moiety according to formula (I);
b. positioned 2, 3 or 4 nucleosides along from the 3′ end of the dinucleotide moiety according to formula (I);
c. directly attached to the 5′ end of the dinucleotide moiety according to formula (I); and/or
d. positioned 2, 3 or 4 nucleosides along from the 5′ end of the dinucleotide moiety according to formula (I).

Suitably, the at least one phosphorothioate linker is:
directly attached to the 3′ end of the dinucleotide moiety according to formula (I); and/or
directly attached to the 5′ end of the dinucleotide moiety according to formula (I).

A phosphorothioate linkage can be represented by either of the following structures:

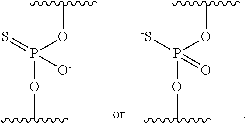

In some embodiments, it may be that all phosphodiester Inkages in the oligonucleotide are replaced with a phosphorothioate linkage. In some embodiments however, some phosphodiester Inkages may be present in the oligonucleotide.

Suitably, more than 75% of the phosphodiester Inkages in the oligonucleotide are replaced with a phosphorothioate linkage. More suitably, more than 90% of the phosphodiester Inkages in the oligonucleotide are replaced with a phosphorothioate linkage. In some embodiments 100% of the phosphodiester Inkages in the oligonucleotide are replaced with a phosphorothioate linkage.

### The Locked Nucleoside

Locked nucleic acids are well known in the art. Any suitable locked nucleoside may be used in the present invention.

The locked nucleic acid may be at a terminal position or may be located centrally.

Typically, the locked nucleoside has the general structure shown below:

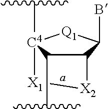

wherein:
   Q₁ is selected from CR^{p}R^{q}, O, S or NR^{a}, wherein R^{p} and R^{q} are each independently selected from H, (1-4C)alkyl or halo, and R^{a} is selected from hydrogen or (1-4C)alkyl;
   B′ is a nucleobase or nucleobase analogue; and
   either
      a) one of X₁ and X₂ is (CR^{a}R^{b})ₓ (where x is selected from 1 or 2) and the other is selected from CR^{a1}R^{b1}, O, NR^{c} or S;
         wherein each of R^{a}, R^{b}, R^{a1} and R^{b1} are independently selected from hydrogen, (1-2C)alkyl, hydroxy, amino, halo or mercapto; and R^{c} is selected from hydrogen or a (1-6C)alkyl; or
      b) one of X₁ and X₂ is O and the other is NR^{c}.

### Preferred Embodiments of the Invention

In another embodiment, the oligonucleotide of the present invention comprises at least one inter-nucleoside linkage which is a phosphorothioate linker moiety, and a moiety of the formula:

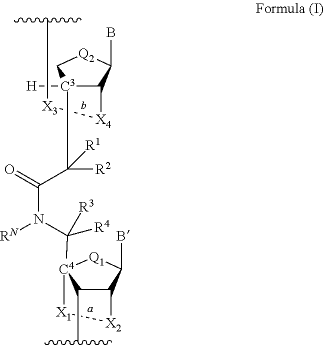

wherein:
C³ is a 3′ carbon;
C⁴ is a 4′ carbon;
Q₁ is selected from CR^{p}R^{q}, O, S or NR^{a}, wherein R^{p} and R^{q} are each independently selected from H, (1-4C)alkyl or halo and R^{a} is selected from hydrogen or (1-4C)alkyl; Q₂ is selected from CR^{p}R^{q}, O, S or NR^{a}, wherein R^{p} and R^{q} are each independently selected from H, (1-4C)alkyl or halo and R^{a} is selected from hydrogen or (1-4C)alkyl; B and B′ are each independently a nucleobase;
bonds a and b are either both present, or one of bonds a and b is absent; either:
   a) if bond a is present, one of X₁ and X₂ is (CR^{a}R^{b})ₓ (where x is selected from 1 or 2) and the other is selected from CR^{a1}R^{b1}, O, NR^{c} or S; or
   b) if bond a is present, one of X₁ and X₂ is O and the other is NR^{c}; or
   c) if bond a is absent, one of X₁ and X₂ is H and the other is selected from H, C₁₋₄alkoxy, F, OH, OR^{c}, O(CH₂)ₙOR^{c} (where n is selected from 1, 2 or 3) or NH₂;
   wherein each of R^{a}, R^{b}, R^{a1} and R^{b1} are independently selected from hydrogen, (1-2C)alkyl, hydroxy, amino, halo or mercapto; and R^{c} is selected from hydrogen or a (1-6C)alkyl;
either:
   a) if bond b is present, one of X₃ and X₄ is (CR^{d}R^{e})_{y} (wherein y is selected from 1 or 2) and the other is selected from CR^{d1}R^{e1}, O, NR^{f} or S; or
   b) if bond b is present, one of X₃ and X₄ is O and the other is NR^{f};
   c) if bond b is absent, one of X₃ and X₄ is H and the other is selected from H, C₁₋₄alkoxy, F, OH, OR^{f}, O(CH₂)ₘOR^{f} (where m is selected from 1, 2 or 3) or NH₂;
   wherein each of R^{d}, R^{e}, R^{d1} and R^{e1} are independently selected from hydrogen, (1-2C)alkyl, hydroxy, amino, halo or mercapto; and
   R^{f} is selected from hydrogen or a (1-6C)alkyl;
R¹ and R² are each independently selected from hydrogen, (1-2C)alkyl, hydroxy, amino or halo;
R³ and R⁴ are each independently selected from hydrogen, (1-2C)alkyl, hydroxy, amino or halo; and
R^{N} is selected from hydrogen or (1-2C)alkyl.

Particular oligonucleotides of the invention include, for example, oligonucleotides comprising a moeity formula I, or pharmaceutically acceptable salts and/or solvates thereof, wherein, unless otherwise stated, each of Q₁, Q₂, bond a, bond b, X₁, X₂, X₃, X₄, R¹, R², R³, R⁴ and R^{N}, and any associated substituent groups has any of the meanings defined hereinbefore or in any of paragraphs (1) to (76) hereinafter:—
(1) Q₁ is selected from CH₂, CF₂, O or S.
(2) Q₁ is O or S.
(3) Q₁ is O.
(4) if bond a is present, then one of X₁ and X₂ is CR^{a}R^{b} and the other is selected from CR^{a1}R^{b1}, O, NR^{c} or S; or
   if bond a is absent, one of X₁ and X₂ is H and the other is selected from H, C₁₋₄alkoxy, F, OH, OR^{c}, O(CH₂)ₙOR^{c} (where n is selected from 1, 2 or 3) or NH₂; wherein:
      each of R^{a}, R^{b}, R^{a1} and R^{b1} are independently selected from hydrogen, (1-2C)alkyl, hydroxy, amino or halo; and
      R^{c} is selected from hydrogen or (1-6C)alkyl;
(5) if bond a is present, then one of X₁ and X₂ is CR^{a}R^{b} and the other is selected from O, NR^{c} or S; or
   if bond a is absent, one of X₁ and X₂ is H and the other is selected from H, C₁₋₄alkoxy, F, OH, OR^{c}, O(CH₂)ₙOR^{c} (where n is an integer selected from 1 or 2) or NH₂;
      wherein:
      R^{a} and R^{b} are independently selected from hydrogen, (1-2C)alkyl, hydroxy, amino or halo; and
      R^{c} is selected from hydrogen or (1-2C)alkyl;
(6) if bond a is present, one of X₁ and X₂ is CR^{a}R^{b} and the other is selected from O, NR^{c} or S; or
   if bond a is absent, one of X₁ and X₂ is H and the other is selected from H, methoxy, F, OH, O(CH₂)₂OMe or NH₂;
      wherein
      R^{a} and R^{b} are independently selected from hydrogen, methyl or fluoro; and
      R^{c} is selected from hydrogen or a methyl;
(7) if bond a is present, X₁ is CR^{a}R^{b} and X₂ is selected from O, NR^{c} or S; or if bond a is absent, X₁ is H and X₂ is selected from H, methoxy, F, OH, O(CH₂)₂OMe;
   wherein:
   R^{a} and R^{b} are independently selected from hydrogen or methyl, and
   R^{c} is selected from hydrogen or methyl.
(8) if bond a is present, X₁ is CH₂ and X₂ is O; or
   if bond a is absent, X₁ is H and X₂ is H or OH.
(9) Q₂ is selected from CH₂, CF₂, O or S.
(10) Q₂ is O or S.
(11) Q₂ is O.
(12) if bond b is present, then one of X₃ and X₄ is CR^{d}R^{e} and the other is selected from CR^{d1}R^{e1}, O, NR^{f} or S; or
   if bond b is absent, one of X₃ and X₄ is H and the other is selected from H, C₁₋₄alkoxy, F, OH, OR^{f}, O(CH₂)ₘOR^{f} (where m is selected from 1, 2 or 3) or NH₂;
      wherein:
      each of R^{d}, R^{e}, R^{d1} and R^{e1} are independently selected from hydrogen, (1-2C)alkyl, hydroxy, amino or halo; and
      R^{f} is selected from hydrogen or (1-6C)alkyl.
(13) if bond b is present, then one of X₃ and X₄ is CR^{d}R^{e} and the other is selected from O, NR^{c} or S; or
   if bond b is absent, one of X₃ and X₄ is H and the other is selected from H, C₁₋₄alkoxy, F, OH, OR^{f}, O(CH₂)ₘOR^{f} (where m is an integer selected from 1 or 2) or NH₂;
      wherein:
      R^{d} and R^{e} are independently selected from hydrogen, (1-2C)alkyl, hydroxy, amino or halo; and
      R^{f} is selected from hydrogen or (1-2C)alkyl.
(14) if bond b is present, one of X₃ and X₄ is CR^{d}R^{e} and the other is selected from O NR^{c} or S; or
   if bond b is absent, one of X₃ and X₄ is H and the other is selected from H, methoxy, F, OH, O(CH₂)₂OMe or NH₂;
      wherein
      R^{d} and R^{e} are independently selected from hydrogen, methyl or fluoro; and
      R^{f} is selected from hydrogen or a methyl.
(15) if bond b is present, X₃ is CR^{d}R^{e} and X₄ is selected from O, NR^{c} or S; or if bond b is absent, X₃ is H and X₄ is selected from H, methoxy, F, OH, O(CH₂)₂OMe;
   wherein:
   R^{d} and R^{e} are independently selected from hydrogen or methyl, and
   R^{f} is selected from hydrogen or methyl.
(16) if bond b is present, X₃ is CH₂ and X₄ is O; or
   if bond b is absent, X₃ is H and X₄ is H or OH.
(17) R¹ and R² are each independently selected from hydrogen or methyl.
(18) R¹ and R² are hydrogen.
(19) R³ and R⁴ are each independently selected from hydrogen or methyl.
(20) R³ and R⁴ are hydrogen.
(21) R^{N} is selected from hydrogen or methyl.
(22) R^{N} is hydrogen.
(23) Either:
   bond a is present and bond b is absent;
   bond b is present and bond a is absent; or
   both bond a and b are present.
(24) Bond a is present and bond b is absent.
(25) Bond b is present and bond a is absent.
(26) Both bond a and b are present.

Suitably, Q₁ is as defined in any one of paragraphs (1) to (3). Most Suitably, Q₁ is as defined in paragraph (3).

Suitably, X₁ and X₂ are as defined in any one of paragraphs (4) to (8). More suitably, X₁ and X₂ are as defined in any one of paragraphs (6) to (8). Most Suitably, X₁ and X₂ are as defined in paragraph (8).

Suitably, Q₂ is as defined in any one of paragraphs (9) to (11). Most Suitably, Q₂ is as defined in paragraph (11).

Suitably, X₃ and X₄ are as defined in any one of paragraphs (12) to (16). More suitably, X₃ and X₄ are as defined in any one of paragraphs (14) to (16). Most Suitably, X₃ and X₄ are as defined in paragraph (16).

Suitably, R¹ and R² are as defined in paragraph (17) or (18). Most Suitably, R¹ and R² are as defined in paragraph (18).

Suitably, R¹ and R² are as defined in paragraph (17) or (18). Most Suitably, R¹ and R² are as defined in paragraph (18).

Suitably, R^{N} is as defined in paragraph (21) or (22). Most Suitably, R^{N} is defined in paragraph (22).

Bonds a and b are as defined in any one of paragraphs (23) to (26). Most suitably, bonds a and b are as defined in paragraph (26).

In the oligonucleotides according to Formula (I), it may be that:
both of bonds a and b are present, or
only one of bonds a and b is present,
thus the oligonucleotide comprises a moiety of Formula (Ia), (Ib) or (Ic), shown below:

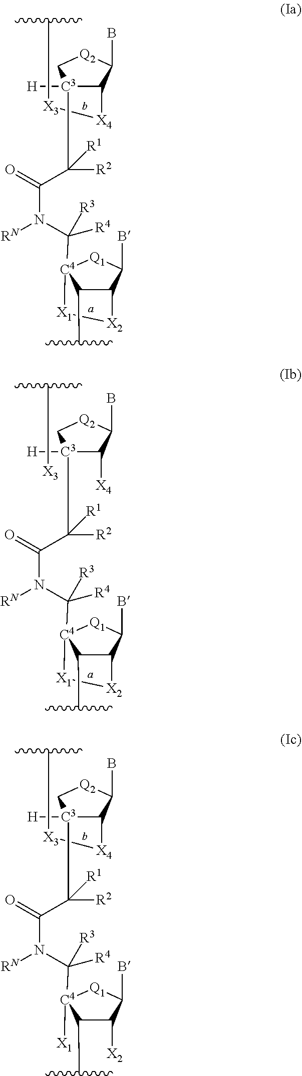

wherein C³, C⁴, Q₁, Q₂, B, B′, X₁, X₂, X₃, X₄, R¹, R², R³, R⁴ and R^{N} are as defined herein.

In a particular group of oligonucleotides of the present invention, both of bonds a and b are present, thus the oligonucleotide comprises a moiety of Formula (Ia) below:

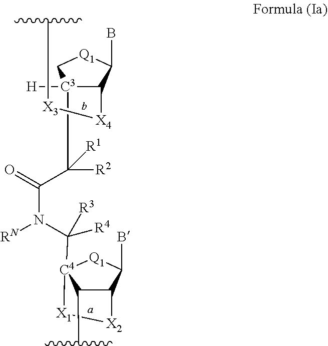

wherein C³, C⁴, Q₁, Q₂, B, B′, X₁, X₂, X₃, X₄, R¹, R², R³, R⁴ and R^{N} are as defined herein.

In a particular group of oligonucleotides of the present invention, at least one phosphorothioate linkage is adjacent to the moeity of Formula (I), thus the oligonucleotide comprises a moiety of Formula (IIa) or (IIb) below:

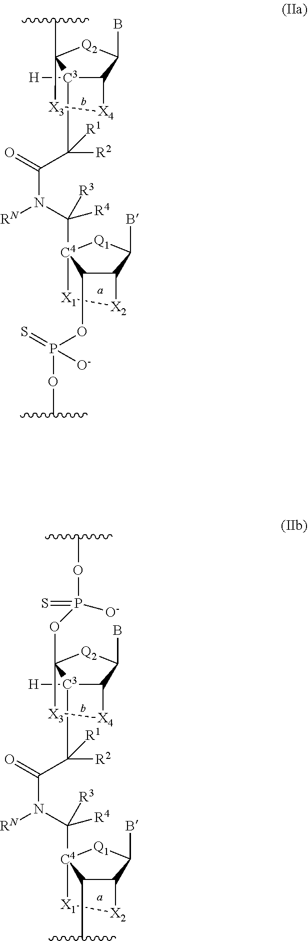

wherein C³, C⁴, Q₁, Q₂, B, B′, X₁, X₂, X₃, X₄, R¹, R², R³, R⁴ and R^{N} are as defined herein.

In a particular group of oligonucleotides of the present invention, at least one phosphorothioate linkage is adjacent to the moeity of Formula (I), and both of bonds a and b are present, thus the oligonucleotide comprises a moiety of Formula (IIc) or (IId) below:

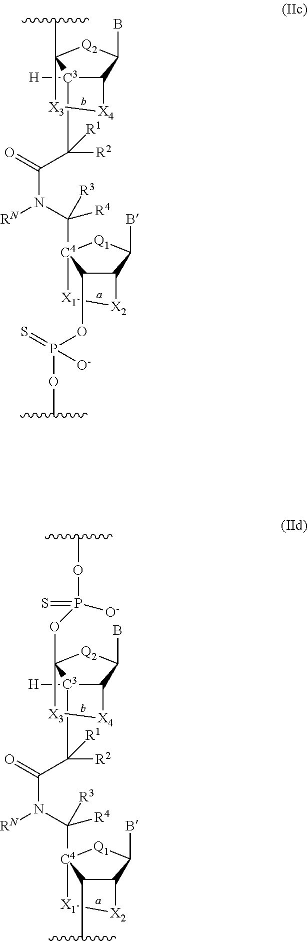

wherein C³, C⁴, Q₁, Q₂, B, B′, X₁, X₂, X₃, X₄, R¹, R², R³, R⁴ and R^{N} are as defined herein.

In a particular group of oligonucleotides of the present invention, at least one phosphorothioate linkage is adjacent to the moeity of Formula (I), both of bonds a and b are present, X₁ is CH₂, X₂ is O, X₃ is CH₂, X₂ is O, Q₁ is O, Q₂ is O, and R¹, R², R³ and R⁴ are hydrogen, thus the oligonucleotide comprises a moiety of Formula (IIe) or (IIf) below:

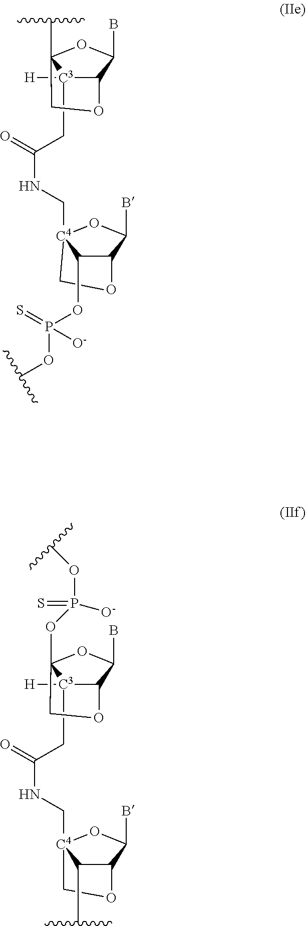

wherein B and B′ are as defined herein.

The oligonucleotides of the present invention will also comprise further nucleotides as part of the oligonucleotide chain. Such nucleotides may include an unmodified or modified sugar moiety as part of the nucleoside. Sugar modified nucleosides are known to the skilled person. The oligonucleotides of the present invention may therefore comprise one or more modified sugar moieties in the sequence (e.g. a 2′OMe sugar).

Suitable nucleosides in the oligonucleotide may have the structural formula shown below:

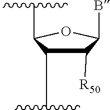

wherein B″ is a nucleobase and R₅₀ is selected from H, C₁-alkoxy, F, OH, OR^{g}, O(CH₂)ₚOR⁹ (where p is selected from 1, 2 or 3) or NH₂, wherein R⁹ is selected from hydrogen or a (1-6C)alkyl. Suitably, R⁹ is hydrogen or methyl.
Suitably, R₅₀ is selected from H, OH, OMe, O(CH₂)₂OMe or F. Thus, the sugar moeity of the nucleoside may be modified or unmodified.

More suitably, R₅₀ is selected from H, OH or OMe.

In an “unmodified” sugar moiety, R₅₀ is H (DNA) or OH (RNA). In a “modified” sugar moiety, R₅₀ may be OMe, O(CH₂)₂OMe or F, suitably OMe.

In a particular group of oligonucleotides of the present invention, the oligonucleotide comprises a moiety of Formula (III) below:

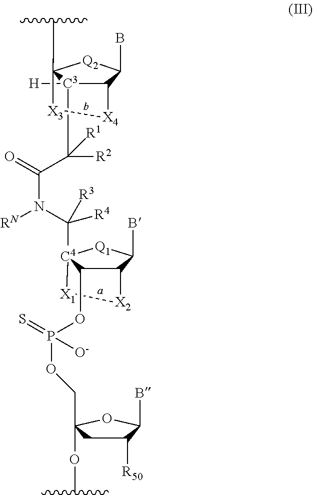

wherein C³, C⁴, Q₁, Q₂, B, B′, X₁, X₂, X₃, X₄, R¹, R², R³, R⁴, R^{N} and R₅₀ are as defined herein.

In a particular group of oligonucleotides of the present invention, in the moiety of formula (III), both of bonds a and b are present, thus the oligonucleotide comprises a moiety of Formula (III):

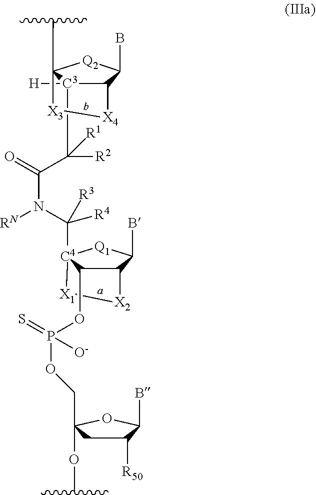

wherein C³, C⁴, Q₁, Q₂, B, B′, X₁, X₂, X₃, X₄, R¹, R², R³, R⁴ and R^{N} are as defined herein.

In a particular group of oligonucleotides of the present invention, in the moiety of formula (III), both of bonds a and b are present, X₁ is CH₂, X₂ is O, X₃ is CH₂, X₂ is O, Q₁ is O, Q₂ is O, and R¹, R², R³ and R⁴ are hydrogen, thus the oligonucleotide comprises a moiety of Formula (lie) or (IIf) below:

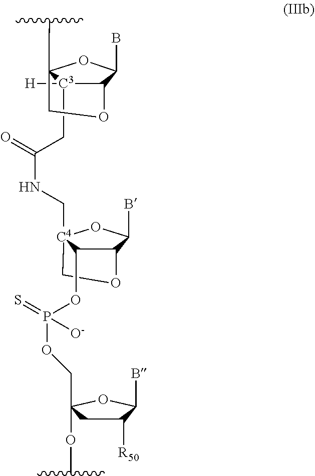

wherein B, B′, B″ and R₅₀ are as defined herein.

### Synthesis

The oligonucleotides of the present invention can be prepared using techniques known in the art.

The preparation of oligonucleotides comprising one or more locked nucleosides in their sequence is known in the art.

Further examples of how to synthesise the oligonucleotides of the present invention are set out in the accompanying examples.

### Uses and Applications

The oligonucleotides of the present invention may be used for a wide variety of applications in fields such as, for example, medicine, genetic testing, gene editing, diagnostics, agriculture, industrial biotechnology, biological research and forensics.

It will be appreciated that certain oligonucleotides of the present invention will have potential therapeutic applications. Examples include antisense RNA oligonucleotides of the present invention as well as certain siRNA and miRNA oligonucleotides.

Another example, is oligonucleotides associated with Clustered Regularly Interspaced Short Palindromic Repeats in combination with CRISPR Associated sequences (CRISPR-Cas) systems, such as for example CRISPR RNA (crRNA), pre-crRNA, tracrRNA and guideRNA (gRNA). Such oligonucleotides find therapeutic utility in the treatment of diseases via e.g. gene therapy as well as in the treatment of infections via selective killing of pathogenic organisms.

In another aspect, the present invention provides an oligonucleotide as defined herein for use in therapy. Examples of potential therapeutic uses of such oligonucleotides include the treatment of cancer, genetic disorders, metabolic disorders, viral infections and bacterial infections. Thus, the present invention provides an oligonucleotide as defined herein a viral infection, cancer, a genetic disorder, a metabolic disease or a bacterial infection.

In another aspect, the present invention provides an oligonucleotide as defined herein for use in the treatment of a viral infection.

In another aspect, the present invention provides an oligonucleotide as defined herein for use in the inhibition of viral messenger RNA.

In another aspect, the present invention provides an oligonucleotide as defined herein for use in the treatment of cancer.

In another aspect, the present invention provides an oligonucleotide as defined herein for use in the inhibition of messenger RNA of a cancer-causing gene.

In another aspect, the present invention provides an oligonucleotide as defined herein for use in the treatment of a genetic disorder, for example diseases caused by loss of function of important endogenous genes, typically in exon-skipping applications (see for example Crooke et al, Antisense technology: A review; JBC Reviews, Vol 296, January 2021), e.g. Duchenne muscular dystrophy, spinal muscular atrophy (SMA).

In another aspect, the present invention provides an oligonucleotide as defined herein for use in the treatment of a metabolically-related disease that is caused by over-production of a specific protein.

In another aspect, the present invention provides an oligonucleotide as defined herein for use in the treatment of a bacterial infection.

In another aspect, the present invention provides a method of treating a viral infection in a subject in need of such treatment, the method comprising administering to said subject a therapeutically effective amount of an oligonucleotide as defined herein, or a pharmaceutically acceptable salt or solvate thereof.

In another aspect, the present invention provides a method of treating cancer in a subject in need of such treatment, the method comprising administering to said subject a therapeutically effective amount of an oligonucleotide as defined herein, or a pharmaceutically acceptable salt or solvate thereof.

In another aspect, the present invention provides a method of treating a genetic disorder in a subject in need of such treatment, the method comprising administering to said subject a therapeutically effective amount of an oligonucleotide as defined herein, or a pharmaceutically acceptable salt or solvate thereof.

In another aspect, the present invention provides a method of inhibiting viral messenger RNA in a subject, the method comprising administering to said subject a therapeutically effective amount of an oligonucleotide as defined herein, or a pharmaceutically acceptable salt or solvate thereof.

In another aspect, the present invention provides a method of treating a bacterial infection in a subject in need of such treatment, the method comprising administering to said subject a therapeutically effective amount of an oligonucleotide as defined herein, or a pharmaceutically acceptable salt or solvate thereof.

In another aspect, the present invention provides a method of treating metabolically-related disease that is caused by over-production of a specific protein in a subject in need of such treatment, the method comprising administering to said subject a therapeutically effective amount of an oligonucleotide as defined herein, or a pharmaceutically acceptable salt or solvate thereof.

In another aspect, the present invention provides a method of inhibiting messenger RNA in a subject, the method comprising administering to said subject a therapeutically effective amount of an oligonucleotide as defined herein, or a pharmaceutically acceptable salt or solvate thereof. Suitably, the messenger RNA is messenger RNA of a cancer causing gene.

The present invention further relates to the use of the oligonucleotides of the present invention as
(i) antisense RNA;
(ii) exon skipping RNA;
(iii) interference RNA (e.g. siRNA or miRNA) or
(iv) an RNA component of a CRISPR-Cas system.

### Illustrative Examples of Oligonucleotides in CRISPR-Cas Systems

In general terms, there are two main classes of CRISPR-Cas systems (Makarova et al. Nat Rev Microbiol. 13:722-736 (2015)), which encompass five major types and 16 different subtypes based on cas gene content, cas operon architecture, Cas protein sequences, and process steps (Makarova et al. Biol Direct. 6:38 (2011); Makarova and Koonin Methods Mol Biol. 1311:47-75 (2015); Barrangou, R. Genome Biology 16:247 (2015)). This classification in either Class 1 or Class 2 is based upon the Cas genes involved in the interference stage.

Class 1 systems have a multi-subunit crRNA-effector complex such as Cascade-Cas3, whereas Class 2 systems have a crRNA-effector complex having a single Cas protein, such as Cas9, Cas12 (previously referred to as Cpf1) and Cas 13a (previously referred to as C2c2). For Type II systems there is a second RNA component tracrRNA which hybridises to crRNA to form a crRNA:tracr RNA duplex, these two RNA components may be linked to form single guide RNA.

RNA components in such CRISPR-Cas systems may be adapted to be an oligonucleotide in accordance with the invention. It would be a matter of routine for a person of ordinary skill in the art to synthesise a crRNA, pre-crRNA, tracrRNA or guideRNA having at least one inter-nucleoside linkage which is a triazole linker moiety between two nucleosides with a locked nucleoside positioned at the 3′ end of the triazole linker moiety, and which retains the desired function of the RNA component (e.g., to guide the crRNA:effector complex to a target site). Standard methods are known in the art for testing whether oligonucleotides of the invention when used as such CRISPR RNA components retain the desired function (e.g. by comparing the desired function to that of a control CRISPR RNA component which has the same nucleosides without any-triazole linker moieties between nucleosides or locked nucleosides).

The term “CRISPR RNA components” or “RNA component of a CRISPR-Cas system” is used herein, as in most CRISPR-Cas systems, the nucleic acid sequences which guide the effector protein(s) to a desired target sequence are RNA components. However, CRISPR hybrid DNA/RNA polynucleotides which can also function to guide effector protein(s) to a desired target site in a DNA or RNA sequence are also known in the art—see for example Rueda et al. (Mapping the sugar dependency for rational generation of a DNA-RNA hybrid-guided Cas9 endonuclease, Nature Communications 8, Article Number: 1610 (2017)). Accordingly, reference to CRISPR RNA components herein may also encompass hybrid RNA/DNA components such as crDNA/RNA, tracrDNA/RNA or gDNA/RNA.

Advantageously the oligonucleotides of the invention may have particular utility in in vivo gene therapy applications. For example, one way of carrying out in vivo therapy using a Type II CRISPR-Cas system involves delivering the Cas9 and tracrRNA via a virus, which can assemble inactive complexes inside of cells. The crRNA can then be administered later to assemble and selectively activate CRISPR/Cas9 complexes, which would then go on to target and edit specific sites in the human genome, such as disease relevant genes (Gagnon and Corey, Proc. Natl. Acad. Sci. USA 112:15536-15537, 2015; Rahdar, et al, Proc. Natl. Acad. Sci. USA 112:E7110-71 17, 2015). For this gene therapy approach to work the crRNA should be extremely resistant to nucleases and cellular degradation, as well as confer high activity and specificity to the assembled CRISPR/Cas9 complex. Hence, the increased stability of the oligonucleotides of the invention to degradation is highly desirable. Alternatively, crRNA:effector complexes (i.e. CRISPR-Cas complexes, such as CRISPR/Cas9) can be assembled in vitro and directly transfected into cells for genome editing (Liang, et al, J. Biotechnol. 208:44-53, 2015; Zuris, et al, Nat. Biotechnol. 33:73-80, 2015). Special transfection reagents, such as CRISPRMAX (Yu, et al, Biotechnol. Lett. 38:919-929, 2016), have been developed for this purpose. Oligonucleotides of the invention when used as crRNAs may improve this approach by offering stability against degradation.

Accordingly, the oligonucleotides of the invention when used as CRISPR RNA components can advantageously be used for the various applications of CRISPR-Cas systems known in the art including: gene-editing, gene activation (CRISPRa) or gene interference (CRISPRi), base-editing, multiplex engineering (CRISPRm), DNA amplification, diagnostics (e.g. SKERLOCK or DETECTR), cell recording (e.g. CAMERA), typing bacteria, antimicrobial applications, synthesising new chemicals etc.

Suitably, in diagnostic applications such as SHERLOCK and DETECTR the oligonucleotides of the invention can be used as RNA components such as the “sacrificial RNA molecules” used to create a signal.

### DESCRIPTION OF THE DRAWINGS

Embodiments of the invention will be described, by way of example only, with reference to the accompanying drawings, which are described below:

FIG. 1: Therapeutic oligonucleotide modifications and the strategy for combining these.
a. Comparing individual therapeutic oligonucleotide modifications. Coding system: ↑=good; →=intermediate; ↓=poor.
b. Overview of this study and the key monomers developed.

FIG. 2: Synthesis of LNA-acid monomers and the structures of other monomers used in this study. a. Synthesis of the DMT-protected LNA ethanoic acids 9a-e. b. X-ray crystal structures confirming the (E)-configuration in 4 and the stereochemistry at the 3′-carbon in 5. c. Phosphoramidites 10⁵⁰ and 11 (commercially available) and the DMT-protected 3′-ethanoic acid DNA-monomer 12^{51,52} used to synthesise oligonucleotides.

FIG. 3: Solid-phase synthesis of amide-phosphodiester chimeras. Dashed lines indicate presence or absence of 2′-4′-methylene bridge.

FIG. 4: Denaturing polyacrylamide gel electrophoresis (PAGE) analysis of modified ONs after incubation in FBS:PBS (1:1). t=incubation time in hours. Underlined bases indicate a locked sugar and * is an amide bond in place of a phosphodiester.

FIG. 5: Structures of amide and LNA-amide modified DNA:RNA duplexes. a. Structural identity of amide and LNA-amide modifications and the torsion angles of the amide backbone (5′ε→3′δ). Pink steps show the modification position (left) and the overlay of all structure shows clear similarities (right). b. Backbone overlay comparing the LNA-amide-LNA step in ON30^{xLNA-Am-LNA} (purple) with the phosphodiester in ON26^{xDNA} (grey). c. Overlay of all amide backbones with or without LNA modifications.

FIG. 6. LNA-flanked amides increase the gymnotic delivery and activity of ONs that contain PS linkages. In addition, they retain exon-skipping activity and are less toxic in combination with LF2000. In all cases luciferase activity was measured and normalised to both protein quantity and untreated cells. Experimental details are given in the Example Section. a. HeLa pLuc/705 cells were transfected at 100 nM ON using LF2000. b. Dose response for active ONs in a. c. Cells were treated with ONs without transfection agent. d. Viability of the HeLa pLuc/705 cells following lipofection with ONs using LF2000 determined using WST-1 assay. e. Change in cell viability after treatment with 200 nM ONs complexed with LF2000 compared to untreated, scale bar=200 m. For all graphs (a-d) Data are means±SD and values for ON18^{2′OMe/4LAL/13PS} and ON20^{2′OMe/17PS} were compared by t test analysis. ns represents P>0.05, * represents P<0.05, ** represents P<0.01, *** represents P<0.001. Each dot represents one replicate (n=3). ON31^{2′OMe/17PS scrambled}: CCUCAUUCACUCGAUUCA. In FIG. 6a, the top listed ON in the legend (ON31) corresponds to the left most bar, the bottom listed ON in the legend (ON20). This ordering continues for each concentration in FIGS. 6b and 6c.

FIG. 7: Proposed neighbouring group participation accounting for the facile displacement of the 5′-mesyl by a hydroxide. Here the carboxylate displaces the 5′-mesyl forming a lactone which is subsequently opened by hydrolysis.

FIG. 8: Synthesis route used for 5′-amino LNA phosphoramidite 10 via the synthesis for S8 reported by Koskin et al.² and how it compares with the route previously reported by Obika et al.³ We have previously reported the synthesis of S14 and S11¹, but have now reduced the equivalents of NaN₃ from 7 to 2 during the S_{N}2 step and have identified conditions to reduce the azide and cleave the benzyl in a single step, improving the scalability of the synthesis.

FIG. 9. UV melting studies for modified ONs against complementary DNA (ON7) (Supplementary Table 2). Left) Representative UV melting curves measured using 3 μM of each ON in pH 7.0 phosphate buffer containing 200 mM NaCl; Right) 1^{st} derivative of melting curves.

FIG. 10. UV melting studies for modified ONs against complementary RNA (ON8) (Supplementary Table 2). Left) Representative UV melting curves measured using 3 μM of each ON in pH 7.0 phosphate buffer containing 200 mM NaCl; Right) 1^{st} derivative of melting curves.

FIG. 11. UV melting studies for modified ONs against RNA with a C mismatch 5′ of the amide (ON9) (Supplementary Table 2). Left) Representative UV melting curves measured using 3 μM of each ON in pH 7.0 phosphate buffer containing 200 mM NaCl; Right) 1^{st} derivative of melting curves.

FIG. 12. UV melting studies for modified ONs against RNA with a C mismatch 3′ of the amide (ON10) (Supplementary Table 2). Left) Representative UV melting curves measured using 3 μM of each ON in pH 7.0 phosphate buffer containing 200 mM NaCl; Right) 1^{st} derivative of melting curves.

FIG. 13. UV melting studies for modified ONs against RNA with a G mismatch 5′ of the amide (ON11) (Supplementary Table 2). Left) Representative UV melting curves measured using 3 μM of each ON in pH 7.0 phosphate buffer containing 200 mM NaCl; Right) 1^{st} derivative of melting curves.

FIG. 15. UV melting studies for modified ONs against RNA with a G mismatch 3′ of the amide (ON12) (Supplementary Table 2). Left) Representative UV melting curves measured using 3 μM of each ON in pH 7.0 phosphate buffer containing 200 mM NaCl; Right) 1^{st} derivative of melting curves.

FIG. 15. UV melting studies comparing 0, 1 and 4 additions of LNA-amide against a complementary DNA target (ON21) (Supplementary Table 3). Left) Representative UV melting curves measured using 3 μM of each ON in pH 7.0 phosphate buffer containing 200 mM NaCl; Right) 1^{st} derivative of melting curves.

FIG. 16. UV melting studies comparing 0, 1 and 4 additions of LNA-amide against a complementary RNA target (ON22) (Supplementary Table 3). Left) Representative UV melting curves measured using 3 μM of each ON in pH 7.0 phosphate buffer containing 200 mM NaCl; Right) 1^{st} derivative of melting curves.

FIG. 17. UV melting studies for modified ONs against truncated DNA target ON23 (Supplementary Table 3). Left) Representative UV melting curves measured using 3 μM of each ON in pH 7.0 phosphate buffer containing 200 mM NaCl; Right) 1^{st} derivative of melting curves.

FIG. 18. UV melting studies for modified ONs against truncated RNA target ON24 (Supplementary Table 3). Left) Representative UV melting curves measured using 3 μM of each ON in pH 7.0 phosphate buffer containing 200 mM NaCl; Right) 1^{st} derivative of melting curves.

FIG. 19. Denaturing polyacrylamide gel electrophoresis (PAGE) analysis of modified ONs after incubation in FBS:PBS (1:1). t=incubation time in hours. Underlined bases indicate a locked sugar and * is an amide bond in place of a phosphodiester.

FIG. 20. Example of crystals obtained from vapor diffusion.
A) ON26^{xDNA}:ON27^{xRNA}
B) ON29^{xLNA-Am-DNA}:ON27^{xRNA}, C) ON30^{xLNA-Am-LNA}:ON27^{xRNA}

FIG. 21. A) Hydrogen-bonding interactions of the 7 A-T, 8 A-T base pair steps surrounding the site of modification. Unmodified (ON26^{xDNA}), amide (ON28^{xDNA-Am-DNA}), LNA-Am (ON29^{xLNA-Am-DNA}) and LNA-Am-LNA (ON30^{xLNA-Am-LNA}). Modifications are contained within the DNA strand (left side) and hydrogen bond distances between Watson-Crick base pairs are shown. B) Overlay of above structures showing base pair step structural similarity.

FIG. 22. Pseudorotation vs torsion angles of angle δ used to define sugar pucker conformations. Torsion data points were calculated using w3DNA 2.0 software and each point represents a single sugar conformation within a corresponding duplex. Each duplex has 20 data points and the clustering of these points can be interpreted to determine duplex form. The theoretical torsion angle is represented by equation (δ=40 cos(P+144)+120). In general, A-form duplexes have consistent pseudorotations 0-60° known as 3′-endo conformation. B-form duplexes have a larger distribution of pseudorotations 0-240°. Legend) Unmodified=ON26^{xDNA}:ON27^{xRNA}; LNA-Am-LNA=ON30^{xLNA-Am-LNA}:ON27^{xRNA}; Amide=ON28^{xDNA-Am-DNA}:ON27^{xRNA}; LNA-Am=ON29^{xLNA-Am-DNA}: ON27^{xRNA}.

FIG. 23. Torsion angle comparison between amide modified backbones. Specific torsions are given by their respective symbol (ε→δ, right). Modified backbones are located at the 7A-T base pair step. Also included is the 7A-T phosphodiester step from the unmodified duplex. In general, all amide backbones, regardless of LNA modification have similar torsion angles. Legend) Unmodified=ON26^{xDNA}:ON27^{xRNA}; LNA-Am-LNA=ON30^{xLNA-Am-LNA}:ON27^{xRNA}; Amide=ON28^{xDNA-Am-DNA}:ON27^{xRNA}; LNA-Am=ON29^{xLNA-Am-DNA}:ON27^{xRNA}.

FIG. 24. LNA-flanked amides increase the gymnotic delivery and activity of ONs that contain PS linkages. Luciferase activity was measured and normalised to both protein quantity and untreated cells.

FIG. 25. Amount of protein per well 24 h after pLuc705 HeLa cells were transfected with ONs using lipofectamine 2000 (LF2000) at the concentrations indicated. A significant drop in protein was observed at higher concentrations indicating toxicity. Higher protein was observed for the amide modified ON, suggesting that LNA-flanked amides reduce toxicity of ONs transfected with LF2000.

FIG. 26. Microscope images 24 h after transfection with LF2000 at the concentrations indicated. The cell morphology suggests that addition of an amide reduces the toxicity of Th-ONs transfected with LF2000 at higher concentrations. Scale bars represent 400 m.

### EXAMPLES

### Summary

Therapeutic oligonucleotides hold great promise against currently untreatable diseases, but are hampered by poor cellular uptake and limited bioavailability.

We investigated if combining the amide linkage (AM1) with locked-nucleic acid (LNA)^{30, 31} and phosphorothioate would overcome the specific limitations of each modification individually (FIG. 1), and produce ‘reduced-charge’ oligonucleotides (ONs) with high target affinity, superior cell uptake and nuclease resistance. In this study we found that as AM1 is a closer analogue of the phosphodiester⁴², surrounding it with 5′- and 3′-LNA could significantly improve duplex stability. This would allow us to incorporate multiple additions of AM1 into a therapeutic oligonucleotide without loss of target affinity.

We show that antisense oligonucleotides containing artificial amide linkages flanked with locked nucleic acid (LNA) within a phosphorothioate backbone have improved cellular uptake, RNA target affinity, nuclease resistance and potency.

To construct such oligonucleotides, analogues of LNA (A, T, C, 5-methyl-C and G), where the 3′—OH group is replaced with an ethanoic acid group, were synthesised in 8 steps, comparable with LNA phosphoramidite synthesis. These were coupled to 5′-amino groups in growing oligonucleotides during standard solid-phase assembly to form inter-nucleoside amide bonds in high yield. X-ray crystal structures of the modified oligonucleotides hybridised to complementary RNA show that the artificial backbone causes minimal structural deviation. 2′OMe phosphorothioate splice-switching oligonucleotides containing just four LNA-amide linkages display greatly improved gymnotic activity relative to oligonucleotides lacking amides, highlighting the therapeutic potential of this technology.

Herein is described the synthesis of LNA-amide-phosphorothioate and LNA-amide-phosphodiester chimeric oligonucleotides, we demonstrate duplex stabilisation and mismatch discrimination, and show that oligonucleotides containing this modification are very stable to nucleases. X-ray crystallography studies with complementary RNA show that the LNA-amide combination does not significantly perturb the A-form duplex. In a biological context, we show that LNA-amide ONs containing 2′OMe sugars and PS backbones are highly effective in a cellular exon-skipping (RNA splice modulation) assay and show greatly improved cellular uptake.

### General Synthetic Procedures

### Amide-Modified Oligonucleotide Synthesis

Oligonucleotide segment synthesis. Oligonucleotide synthesis was performed on an Applied Biosystems 394 automated DNA/RNA synthesiser on a 1.0 μmole scale using a standard phosphoramidite cycle of detritylation, coupling, and oxidation. No capping step was used. All β-cyanoethyl phosphoramidite monomers were dissolved in anhydrous MeCN (10% CH₂Cl₂ was added when 2′OMe U phosphoramidite was used) to a concentration of 0.1 M immediately prior to use. 5-(Benzylthio)-1H-tetrazole (BTT) activator (0.3 M) was used with a coupling time of 50 s for normal dA, dG, dC and T phosphoramidites, this was extended to 6 min for 2′OMe and LNA phosphoramidites. Standard iodine oxidiser was used for phosphodiester oligonucleotides. For the phosphorothioate oligonucleotides, 3-ethoxy-1,2,4-dithiazoline-5-one (EDITH) was used as the sulfurisation agent, and the solid support was washed with MeCN after each phosphoramidite coupling before the sulfurisation step. Sulfurisation time was initially 3 min, and after this period fresh EDITH was sent to the synthesis column and left for another 3 min. Further details are given below.

Amino monomer addition. The MMT-protected 5′-amino phosphoramidite monomer (either LNA 10⁵⁰ or commercially available deoxythymidyl 11) was dissolved in anhydrous MeCN to a concentration of 0.1 M immediately prior to use. The same coupling conditions as above were used, but the coupling time was extended to 10 min. The MMT protecting group was cleaved on the Applied Biosystems 394 automated synthesiser using 3% TCA in CH₂Cl₂ with an extended cleavage time of 2 min. The solid support was then washed with acetonitrile on the synthesiser for 3 min. To improve the coupling efficiency in the next step, the solid support was washed with 0.5% (v/v) N-methylmorpholine in DMF (1×1 mL) followed by DMF (3×1 mL).

Amide bond formation on resin (peptide coupling). All amide couplings were performed manually on the synthesis column. A solution with 10 equivalents of acid monomer, 10 equivalents of PyBOP and 30 equivalents of N-methylmorpholine was first prepared in 400 μL of DMF. This was then taken up into a 1 mL syringe and loaded onto the column before a second 1 mL syringe was attached to the other end of the synthesis column. The mixture was agitated every 10 min for 1 h. The columns were then washed with DMF (3×1 mL) followed by MeCN (5×1 mL) and dried by passing argon through the column. The column was then returned to the synthesiser to continue oligonucleotide synthesis.

Cleavage of oligonucleotides from resin, deprotection and purification. LNA-amide containing oligonucleotides were isolated with the final 5′-DMT protecting group still in place (DMT-ON). Following solid phase synthesis, the cyanoethyl groups were removed by a 15 min treatment with 20% diethylamine in MeCN. The resin was then washed with MeCN (5×1 mL) and dried by passing a stream of argon through the synthesis column. The oligonucleotides were cleaved from the solid support and deprotected by heating in concentrated aqueous ammonia in a sealed glass vial at 55° C. for 5 hours. The ammonia was removed under reduced pressure prior to oligonucleotide purification. The DMT-ON oligonucleotides were purified by reverse-phase high performance liquid chromatography (RP-HPLC) and lyophilised. They were then dissolved in 0.5 mL of 80% acetic acid and left for 1 hour at room temperature to remove the DMT group. The solution was neutralised with 0.5 mL of triethylammonium acetate buffer (2 M, pH 7) and the detritylated oligonucleotides were desalted using a NAP-10 column (Cytiva) then freeze dried.

### Results and Discussion

### Synthesis of LNA-Amide Monomers for Oligonucleotide Assembly is Efficient and Scalable

Efficient synthesis of chemically modified oligonucleotides is essential for fundamental studies and therapeutic applications. We have devised a combined solid-phase phosphoramidite coupling/amide bond formation approach which enables the straightforward assembly of oligonucleotides containing LNA-flanked amides, and which could be easily automated and/or adapted by others in the field. In our strategy a maximum of eight monomers are required to make all possible nucleobase sequences containing amide linkages (four carboxylic acids and four amines).

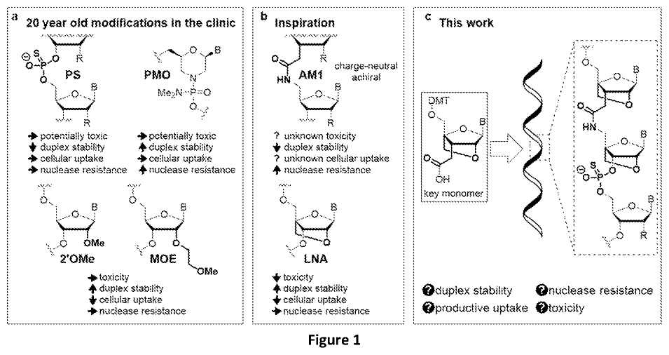

Synthesis of the required 5′-dimethoxytrityl (DMT)-protected 3-ethanoic acid LNA-monomers was achieved in just 8 steps from 1, the same as for standard LNA phosphoramidites following the most commonly cited route⁴³ (FIG. 2a). We first built the sugar with the C3′-ester 5, before addition of the nucleobase. This approach avoids complications associated with forming a C—C bond at the 3′-side of a nucleoside using the Barton-McCombie reaction^{23-25, 44} or the hydrogenation step if a Wittig reaction is used^{45, 46}, which would ultimately limit the range of heterocyclic bases that can be added. Key intermediate 5 was readily prepared as follows on multiple gram scale in 81% overall yield from commercially available 1 without the need for chromatographic separation. Hydrogenolysis of compound 1 afforded alcohol 2 which was subsequently oxidised to give 3. Olefination of 3 with (carbethoxymethylene)triphenylphosphorane (Wittig reaction) selectively yielded 4 as the (E)-stereoisomer (FIG. 2b). Catalytic hydrogenation of 4 using Pd/C and H₂ gave 5 as a single stereoisomer (FIG. 2b). This stereoselectivity was predicted because the 1,2-O-isopropylidene groups on the α-face of furanosyl carbohydrate derivatives direct incoming H₂ to the β-face⁴⁷. Key intermediate 5 was then converted to the 1,2-di-O-acetate glycosyl donor 6 following a procedure reported by Arzel et al.⁴⁸, avoiding the formation of a lactone which occurred when the acetonide was cleaved in the presence of water.

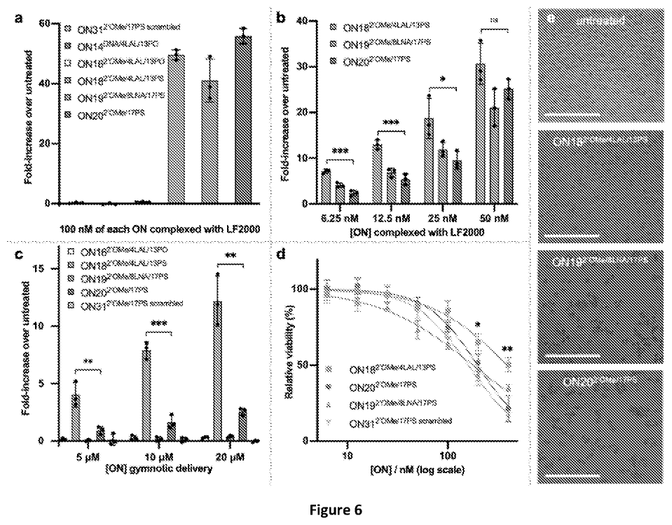

The pathways to each monomer then diverged, with Vorbruggen conditions^{43, 49} utilised for addition of the nucleobases to access 7a-e. Subsequent simultaneous unmasking of the 3′-carboxyl and 2′-hydroxyl groups by treatment with hydroxide, followed by cyclisation to form the 2′-4′-oxymethylene bridge, then 5′-mesyl deprotection, gave the hydroxy-LNA acid compounds 8a-e. The progress of the reaction was rapid; mesyl deprotection using hydroxide ion conventionally requires several days under reflux conditions⁴³. We postulate that the acceleration in rate is due to neighbouring group participation whereby the carboxylate anion displaces the 5′-mesyl group, forming a lactone that is subsequently opened by hydrolysis (FIG. 7). Finally, we treated the resulting hydroxy-acids 8a-e with 4,4′-dimethoxytrityl chloride (DMT-Cl) in pyridine to give the DMT-protected LNA analogue nucleosides 9a-e.

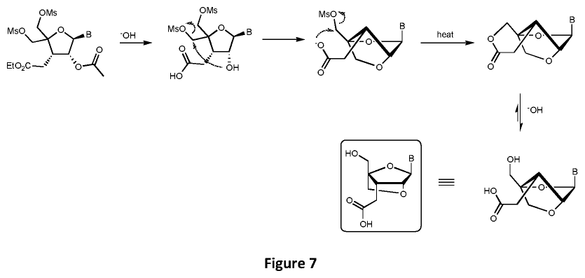

Using this strategy we were able to access all four canonical nucleoside analogues along with the 5-methylcytidine version which is often used in antisense experiments to increase target affinity¹⁶. Additionally, we required phosphoramidites 10⁵⁰ and commercially available 11 with monomethoxytrityl (MMT)-protected 5′-amino groups, and the 5′-DMT-protected thymidine 3′-ethanoic acid monomer 12^{51, 52} (FIG. 2c). Whilst the required 5′-amino LNA phosphoramidite 10 had been previously synthesised⁵⁰, we chose to develop a more efficient route (FIG. 8).

### Amide-Phosphodiester Chimeras can be Synthesised in High Yield and Purity

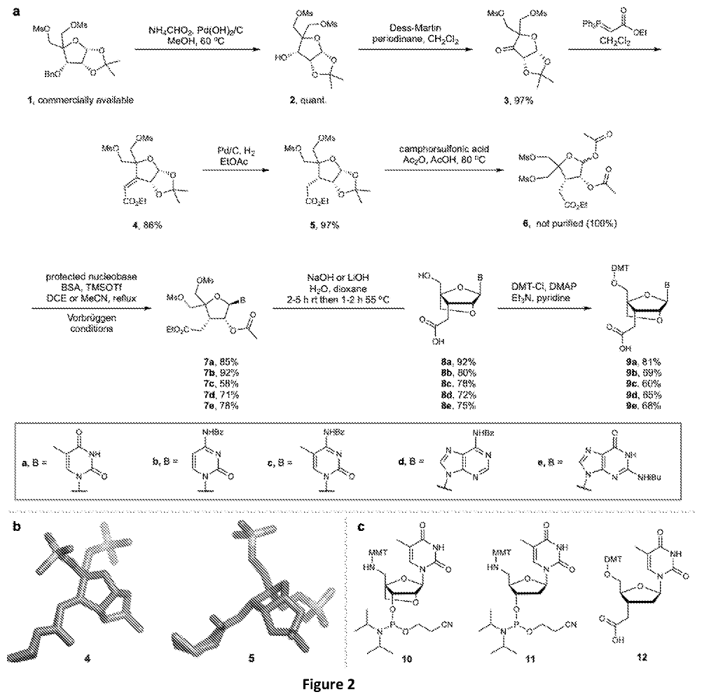

An overview of our oligonucleotide synthesis strategy is shown in FIG. 3. A phosphoramidite monomer with an MMT-protected 5′-amino group, either LNA 10⁵⁰ or deoxythymidyl 11, is added to the oligonucleotide, and the amine is deprotected using trichloroacetic acid (TCA). An LNA-acid (or DNA-acid⁵³) monomer is coupled to the free amine using PyBOP activating agent (benzotriazol-1-yloxytripyrrolidinophosphonium hexafluorophosphate) in the presence of a non-nucleophilic base (N-methylmorpholine) to form the amide bond. Oligonucleotide synthesis is then resumed, starting with the TCA-mediated removal of the DMT group.

The process can be repeated to install multiple non-contiguous amides in the same oligonucleotide. To demonstrate this, DMT-protected LNA acids 9a-e, phosphoramidites 10 and 11, and DNA acid 12^{51, 52} (FIG. 3), were used to synthesise several oligonucleotides, some of which contain multiple additions of LNA-amide, 2′OMe sugars and PS linkages (Supplementary Table 1). In all cases, we obtained the oligonucleotides in high yield and purity. High-performance Liquid chromatography (HPLC) and mass spectral data demonstrated the high incorporation efficiency for the DMT-protected LNA acids 9a-e.

### LNA Sugars Stabilise Duplexes Containing the Artificial Amide DNA Backbone

To evaluate the compatibility of AM1 with LNA, we synthesised ONs with a single amide linkage flanked by either no LNA (ON1^{DNA-Am-DNA}), an LNA 5′ to the amide (ON2^{LNA-Am-DNA}) an LNA 3′ to the amide (ON3^{DNA-Am-LNA}), or LNA on both sides of the amide (ON4^{LNA-Am-LNA}) along with LNA without amide (ON5^{LNA-LNA}) and DNA without amide (ON6^{DNAcontrol}). We compared the duplex denaturation temperatures (Tₘs) after hybridisation with DNA and RNA complementary strands. In the DNA:RNA hybrids, ON2^{LNA-Am-DNA} showed a significant increase in duplex stability (+3.0° C.) compared to the unmodified ON6^{DNAcontrol}, and an increase of +3.4° C. compared to ‘amide only’ ON1^{DNA-Am-DNA}. This supports our hypothesis that LNA can stabilise artificial backbones that are close analogues of canonical phosphodiester linkages. As expected, ON4^{LNA-Am-LNA} in which the amide is surrounded by LNA sugars, gave the greatest increase in stability of the amide modified ONs (+5.1° C.). It is noteworthy that ON2^{LNA-Am-DNA} and ON4^{LNA-Am-LNA} provide the first examples of an LNA sugar with an immediate 3′-non-phosphorus DNA backbone stabilising a duplex.

RNA sequence selectivity of the amide-containing ONs was excellent; ONs 1-4 all showed significant duplex destabilisation when hybridised to an RNA strand with a single mismatched base pair. ON2-4 with the various combinations of LNA and the amide linkage all had lower duplex stability than the LNA alone (ON5^{LNA-LNA}), allowing several amide linkages to be incorporated into an oligonucleotide without producing excessively high duplex stability (Table 2, discussed further below). In summary, an amide linkage flanked by LNA on both sides gives excellent DNA:RNA duplex stabilisation and mismatch discrimination.

In duplexes with DNA targets, ONs with all combinations of LNA and DNA sugars around the amide linkage were very slightly destabilising (between −0.1° C. to −2.6° C.), indicating their selectivity for RNA over DNA.

TABLE 1
Comparison of the melting temperatures (Tₘ) of duplexes containing a single
amide substitution of the phosphodiester backbone flanked by LNA on the 5′, 3′
or both sides within a DNA backbone hybridised to DNA or RNA

| | | DNA | RNA | RNA 5′ | RNA 3′ | RNA 5′ | RNA 3′ |
|---|---|---|---|---|---|---|---|
| | | match | match | C | C | G | G |
| | | Tm | Tm | mismatch | mismatch | mismatch | mismatch |
| | Sequence (5′ → | (ΔTₘ) | (ΔTₘ) | Tₘ (ΔTₘ) | Tm (ΔTₘ) | Tₘ (ΔTₘ) | Tₘ (ΔTₘ) |
| ON | 3′) | ON7 | ON8 | ON9 | ON10 | ON11 | ON12 |
| ON1^{DNA-} | CGACGCT*TGCA | 61.2 | 61.0 | 47.0 | 50.2 | 57.3 | 56.6 |
| ^{Am-DNA} | GC | (−2.3) | (−0.4) | (−14.0) | (−10.8) | (−3.7) | (−4.4) |
| ON2^{LNA-} | CGACGCT*TGCA | 63.1 | 64.4 | 50.2 | 51.0 | 58.5 | 60.3 |
| ^{Am-DNA} | GC | (−0.4) | (+3.0) | (−14.2) | (−13.4) | (−5.9) | (−4.1) |
| ON3^{DNA-} | CGACGCT*TGCA | 60.9 | 63.9 | 50.1 | 52.5 | 58.8 | 56.9 |
| ^{Am-LNA} | GC | (−2.6) | (+2.5) | (−13.8) | (−11.4) | (−5.1) | (−7.0) |
| ON4-^{LNA-} | CGACGCT*TGCA | 63.4 | 66.5 | 53.3 | 55.1 | 59.8 | 60.5 |
| ^{Am-LNA} | GC | (−0.1) | (+5.1) | (−13.2) | (−11.4) | (−6.7) | (−6.0) |
| ON5^{LNA-} | CGACGCTTGCA | 69.6 | 74.1 | 59.7 | 59.9 | 68.6 | 67.3 |
| ^{LNA} | GC | (+6.1) | (+12.7) | (−14.4) | (−14.2) | (−5.5) | (−6.8) |
| ON6^{DNAcontrol} | CGACGCTTGCA | 63.5 | 61.4 | 46.2 | 51.2 | 57.4 | 57.4 |
| | GC | | | (−15.2) | (−10.2) | (−4.0) | (−4.0) |

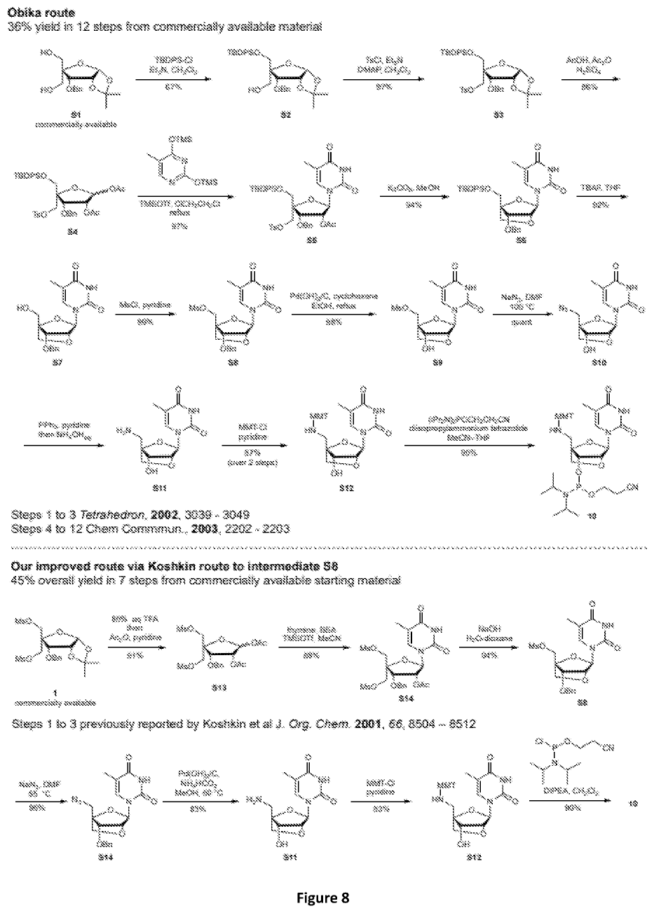

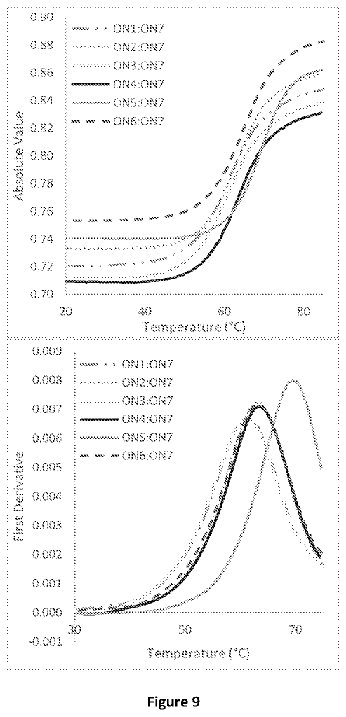

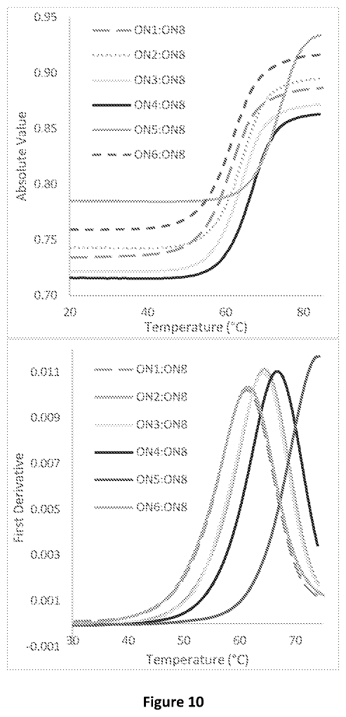

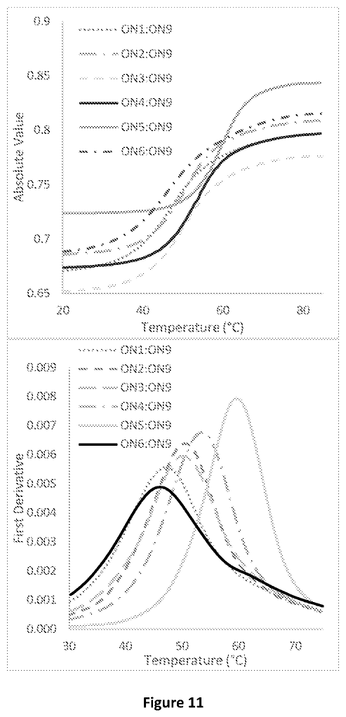

Tₘ values were measured using 3.0 μM concentrations of each oligonucleotide strand in 10 mM phosphate buffer (pH 7.0) containing 200 mM NaCl. T indicates a locked sugar and * is an amide bond in place of a phosphodiester. Tₘ values were calculated as the maximum of the first derivative of the melting curve (A₂₆₀ vs temperature) and reported as the average of at least two independent experiments. ΔTₘ for matched sequences=modified−ON6^{DNAcontrol}; ΔTₘ for mismatched=RNA mismatch−match. Target ON sequences, where an underlined base denotes the mismatch: ON7=GCTGCAAGCGTCG; ON8=GCUGCAAGCGUCG; ON9=GCUGCACGCGUCG; ON10=GCUGCCAGCGUCG; RNA ON11=GCUGCAGGCGUCG. ON12=GCUGCGAGCGUCG. Representative melting curves are given (FIG. 9-12).

The stabilisation induced by the LNA amide combination is cumulative and general (Table 2a). In a different biologically relevant sequence context from above, addition of one amide flanked with LNA to unmodified DNA (ON13^{DNA/1LAL/16PO}) increases duplex stability by 1.8° C., whereas addition of four amides flanked by LNA (ON14^{DNA/4LAL/13PO}) gives an increase of 5.1° C. A much stronger trend is observed for duplexes with RNA, where a single amide flanked by LNA increases the DNA:RNA hybrid Tₘ by 2.2° C., and four LNA-flanked amides increase it by an impressive 13.0° C. This selectivity for RNA over DNA is an important advantage when developing therapeutic oligonucleotides to selectively target RNA.

It was not possible to directly determine the duplex melting temperatures of the 2′OMe oligonucleotides containing multiple LNA amide additions as the duplexes were too stable. Instead, duplex melting temperatures were measured against shorter 10-mer DNA (ON23) and RNA (ON24) targets complementary to the 5′-portion (Table 2b-d). In all cases the combination of LNA and amide greatly increased duplex stability and, as expected, PS linkages reduced the stability of duplexes relative to phosphodiesters.

TABLE 2
Comparison of the relative melting temperatures of duplexes containing 0, 1 or
4 amide linkages flanked by LNA on both sides and hybridised to DNA or RNA

| | | | DNA | RNA |
|---|---|---|---|---|
| ON | Sequence (5′ → 3′) | backbone | Tₘ (ΔTₘ) | Tₘ (ΔTₘ) |
| a | | | | |
| ON13^{DNA/1LAL/16PO} | CCTCTTACC*TCAGTTACA | DNA, PO | 60.9 | 65.8 |
| | | | (+1.8) | (+2.2) |
| ON14^{DNA/4LAL/13PO} | CC*TCT*TACC*TCAGT*TACA | DNA, PO | 64.2 | 76.6 |
| | | | (+5.1) | (+13.0) |
| ON15^{DNA/17PO} | CCTCTTACCTCAGTTACA | DNA, PO | 59.1 | 63.6 |
| b | | | | |
| ON14^{DNA/4LAL/13PO} | CC*TCT*TACC*Tcagt*taca | DNA, PO | 46.5 | 64.3 |
| | | | (+7.0) | (+17.5) |
| ON15^{DNA/17PO} | CCTCTTACCTcagttaca | DNA, PO | 39.5 | 46.8 |
| c | | | | |
| ON16^{2′OMe/4LAL/13PO} | C^{Me}C*TCT*TAC^{Me}C*Tcagt*taca | 2′OMe, | 53.2 | 72.3 |
| | | PO | (+19.1) | (+9.3) |
| ON17^{2′OMe/17PO} | CCUCUUACCUcaguuaca | 2′OMe, | 34.1 | 63.0 |
| | | PO | | |
| d | | | | |
| ON18^{2′OMe/4LAL/13PS} | C^{Me}C*TCT*TAC^{Me}C*Tcagt*taca | 2′OMe, | 48.1 | 67.4 |
| | | PS | (+25.4) | (+10.3) |
| ON19^{2′OMe/8LNA/17PS} | C^{Me}CTCTTAC^{Me}CTcagttaca | 2′OMe, | 59.0 | >80 |
| | | PS | (+36.3) | |
| ON20^{2′OMe/17PS} | CCUCUUACCUcaguuaca | 2′OMe, | 22.7 | 57.1 |
| | | PS | | |

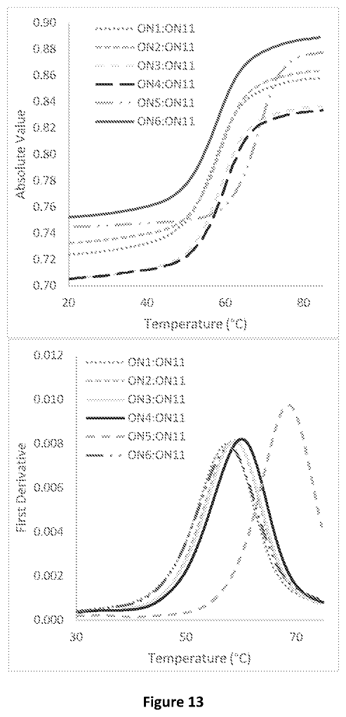

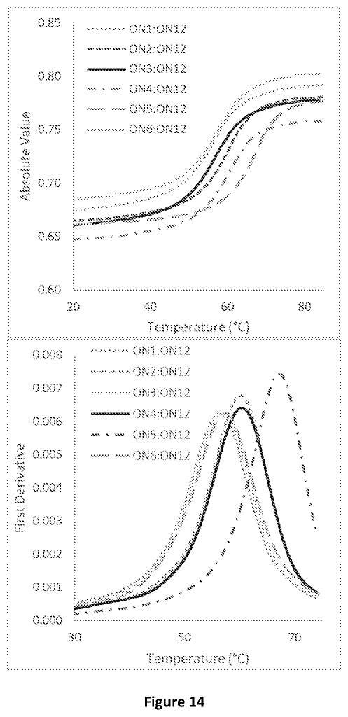

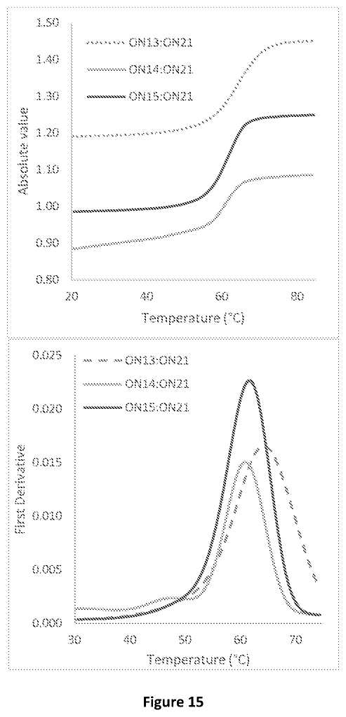

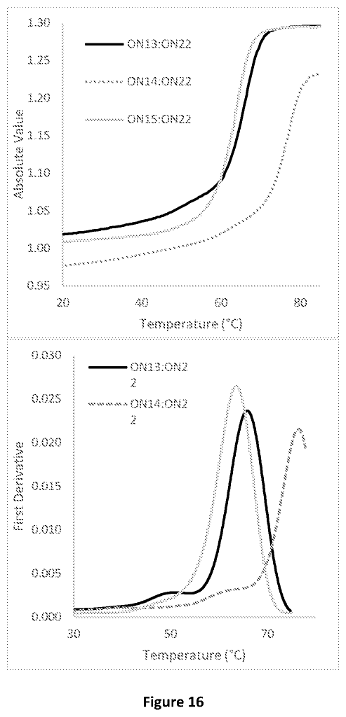

Experimental conditions as in Table 1. a. comparison of DNA ONs against full length targets, b. comparison of DNA ONs against 10-mer targets, c. comparison of 2′OMe/PO ONs, d. comparison of 2′OMe/PS ONs. Backbone: PO=phosphodiester, PS=phosphorothioate, DNA=deoxyribose sugars, 2′OMe=2′OMe RNA sugars. Underlined bases indicate a locked sugar and * indicates an amide bond in place of a phosphodiester. The ON code indicates the number of each backbone linkage where LAL stands for LNA-flanked amide bonds. DNA target (ON21)=TGTAACTGAGGTAAGAGG; RNA target (ON22)=UGUAACUGAGGUAAGAGG. Truncated DNA target (ON23)=AGGTAAGAGG. Truncated RNA target (ON24)=AGGUAAGAGG. ΔTₘ=modified−control. Bases in lowercase italic remain single stranded on duplex formation and do not contribute to Tₘ. Representative melting curves are given (FIG. 15-18).

### The Combination of LNA and Amide Provides Extreme Nuclease Resistance

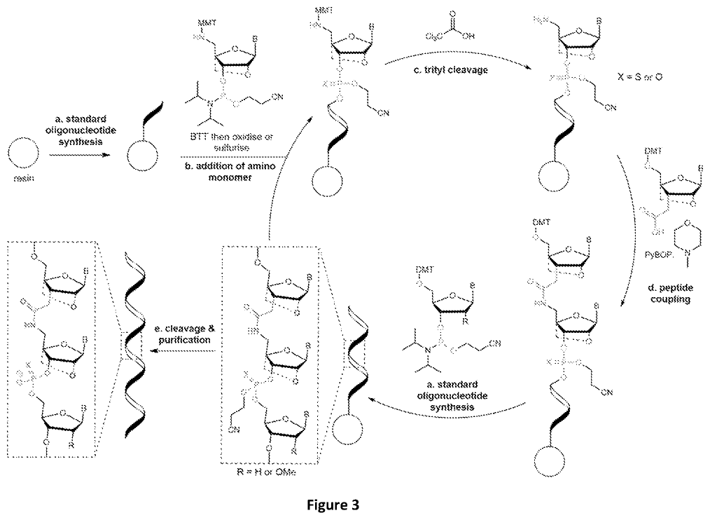

To evaluate whether the combination of LNA and amide confers greater nuclease resistance than LNA alone, we incubated unmodified DNA (ON15^{DNAN17PO}) and DNA with four amide linkages flanked with LNA (ON14^{DNA4LAL/13PO}) in a 1:1 mixture of phosphate buffered saline (PBS) and foetal bovine serum (FBS) to mimic the in vivo environment, and compared it with DNA with the same eight LNA sugars without amide linkages (ON25^{DNA8LNA/17PO}). We sampled aliquots at different time intervals, and analysed them by gel electrophoresis (FIG. 4). The results show that the combination of LNA and amide linkages offer the greatest resistance to nucleases. Both oligonucleotides lacking amide linkages had partially degraded within 1 hour whereas ON14^{DNA/4LAL/13PO} remained intact after 8 hours.

X-Ray Crystallography Reveals that LNA Amide Modifications Cause Minimal Structural Deviation.

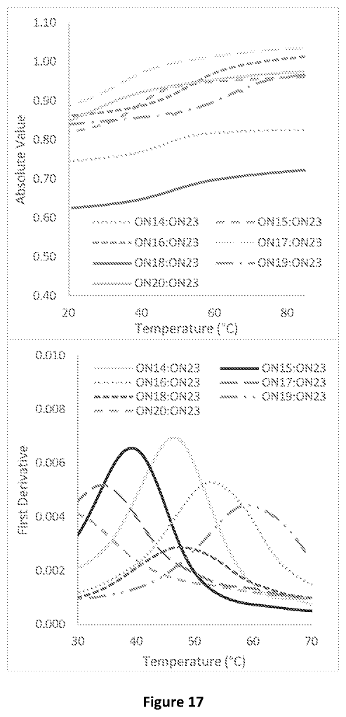

X-ray structures were carried out to determine the effects of LNA and amide modifications on duplex conformation. These are the first crystal structures of DNA:RNA hybrids that contain amide linkages. The X-ray structure of an amide-modified RNA:RNA duplex was determined previously, but this was a self-complementary sequence with amide linkages in both strands. Moreover, the amide linkages were surrounded by multiple mismatched base pairs, an unusual construct that cannot exist outside the solid-state²⁷. The sequence of the LNA-amide modified DNA:RNA hybrid duplexes was based on the corresponding unmodified hybrid PDB 1PJO⁴ (Table 3). Crystals of DNA with a single amide linkage flanked entirely by DNA (ON28^{xDNA-Am-DNA}) an LNA 5′ to the amide (ON29^{xLNA-Am-DNA}), and LNA on both sides of the amide (ON30^{xLNA-Am-LNA}) all hybridised to complementary RNA (ON27^{xRNA}), diffracted to between 2.5-2.8 A resolution (FIG. 19). The structure of the unmodified DNA:RNA duplex (ON26^{xDNA}:ON27^{xRNA}) was obtained for comparison. The data collection and refinement statistics are given in Supplementary Table 2.

TABLE 3
Sequences of oligonucleotides used in
crystallographic studies

| | Sequence (5′ → | |
|---|---|---|
| ON | 3′) | backbone |
| ON26^{xDNA} | CTTTTCTTTG | DNA |
| ON27^{xRNA} | CAAAGAAAAG | RNA |
| ON28^{xDNA-Am-DNA} | CTT*TTCTTTG | DNA |
| ON29^{xLNA-Am-DNA} | CTT*TTCTTTG | DNA |
| ON30^{xLNA-Am-LNA} | CTT*TTCTTTG | DNA |

T indicates a locked sugar and * indicates an amide bond in place of a phosphodiester.

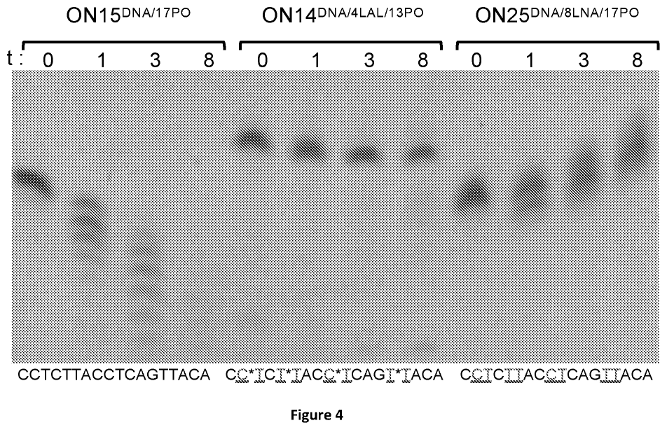

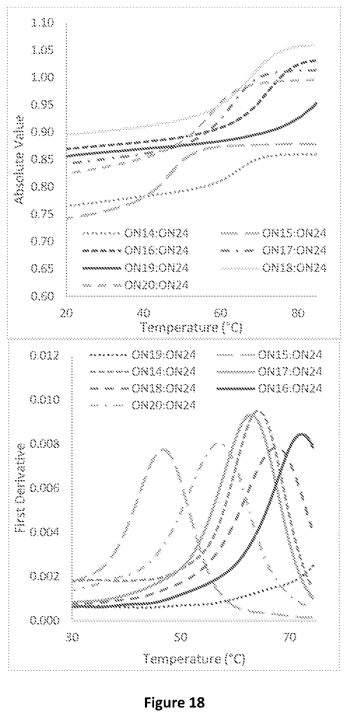

The hybrids all adopt an A-form duplex structure. Importantly, structures of the modified duplexes containing the amide and LNA-amide backbones (FIG. 5a) are consistent with the unmodified duplex (all-atom root-mean-square deviation (RMSD) 0.4 A). Analysis of sugar puckers (FIG. 20) shows that most cluster around the C₃′-endo conformation, a trait associated with A-form duplexes. Inclusion of the amide and LNA-amide modifications has no significant impact on global duplex structure, and a superimposition of all structures reveals that the base pairs and backbones are structurally consistent (FIG. 5a, FIG. 21). Amide and LNA modifications do not cause significant alterations to inter-base hydrogen-bonding distances, and all base pairs adopt a canonical Watson-Crick structure, indicating that any thermodynamic improvements are not due to unusual changes in hydrogen bonding interactions.

In agreement with the amide-DNA:RNA hybrid NMR structure by Rosners²⁸ in which the DNA strand contained multiple amides, our X-ray studies indicate that the amide linkage is a close mimic of the phosphodiester backbone (FIG. 5b). Both are four-atom linkages, hence similar in length, and the amide carbonyl is orientated in the same direction as one of the phosphodiester P—O bonds. Although the resolution of the X-ray structure is insufficient to unambiguously show interactions with water molecules in this region, the amide must interact with surrounding solvent, and the amide and phosphate oxygen atoms can participate in similar solvent hydrogen bonding interactions. However, unlike the phosphate, the amide has only a single hydrogen-bond acceptor. Interestingly, the global hydration of the amide-containing duplex has been shown by NMR to be relatively unchanged compared to the unmodified duplex²⁸.

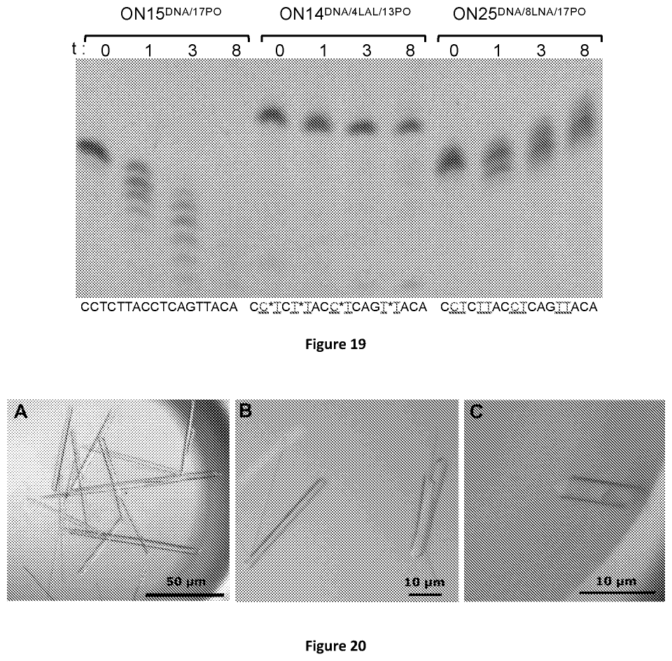

In FIG. 5c, the structures of all amide backbones are overlaid to assess the effects of the LNA modifications. Between each structure, the orientation of the backbone is consistent, directing the amide oxygen into the major groove. Other atomic positions of the backbones also show close similarity, and the presence of 3′-LNA causes no significant distortion. 5′-LNA does however cause some structural displacement; the 5′-sugars in the LNA-amide and LNA-amide-LNA structures are shifted slightly outwards compared to the DNA-amide and unmodified strands. Despite this, the positioning of the amide backbone remains consistent between each structure. The amide adopts the expected trans-conformation, and LNA on the 5′-side of the amide has little effect on backbone torsion angles (FIG. 5c, FIG. 22). In summary, LNA and amide modifications have minimal effect on the duplex structure, and the amides are excellent mimics of natural phosphodiesters.

### The Combination of LNA-Amide and PS Greatly Enhance Gymnotic Delivery and Exon-Skipping Activity

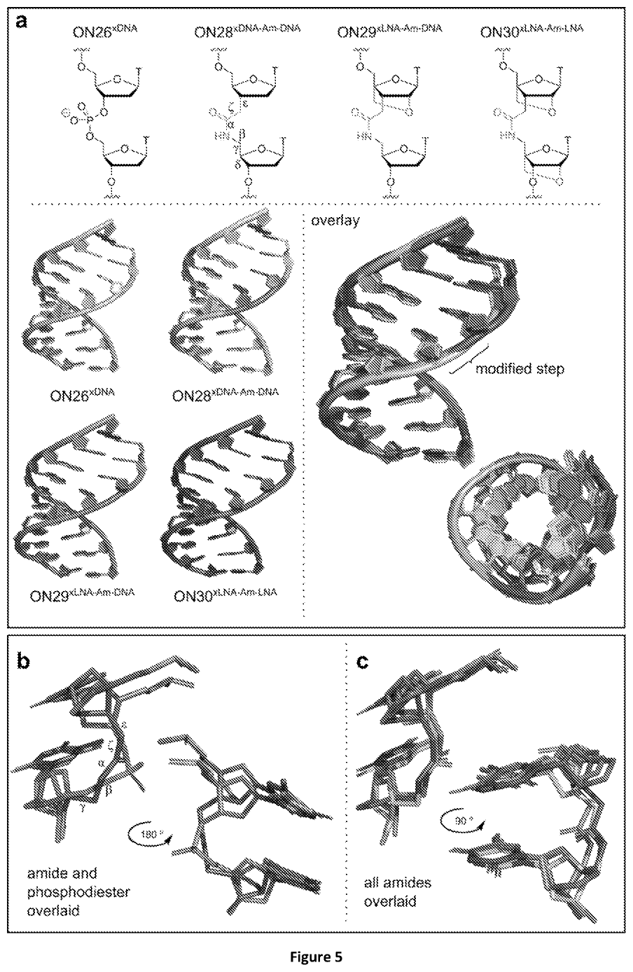

Poor cellular uptake and cellular toxicity remain major obstacles when developing therapeutic oligonucleotide agents. We sought to evaluate the biological activity of the LNA-amide combination using the HeLa pLuc/705 cell line⁵⁵. This cell line carries a luciferase-encoding gene that is interrupted by a mutated ß-globin intron⁵⁵. The mutation creates a 5˜-splice site which in turn activates a cryptic 3′-splice site, resulting in incorrect mRNA splicing and the production of non-functional luciferase. An oligonucleotide that hybridises to the mutant 5′-splice site prevents incorporation of the aberrant intron. This restores the luciferase pre-mRNA splicing pattern to produce functional luciferase, which is quantified by LIuminometry. The oligonucleotides in Table 2 were designed to be complementary to this aberrant splice site. Oligonucleotides ON14^{DNA/4LAL/13PO} ON16^{2′OMe/4LAL/13PO}, and ON18^{2′OMe/4LAL/13PS} have the LNA-amide modification in the same position and were designed to evaluate LNA-amide in combination with the DNA, 2′OMe/phosphodiester, and 2′OMe/phosphorothioate backbones respectively. We decided to evaluate ONs 14 and 16 with phosphodiester backbones in these exon-skipping studies as neither LNA or the amide linkages are compatible with RNase-H^{2,56}, and the LNA-amide modification strongly protects ONs against nuclease degradation. In addition, it allowed us to determine the consequences of the absence of the PS linkages on delivery/activity of LNA-amide ONs. Three controls were included: ON20^{2′OMe/17PS} to determine whether the LNA-amide linkage can improve the biological activity of a therapeutic oligonucleotide⁵⁵, ON17^{2′OMe/17PO} to evaluate the importance of the PS linkage independent of LNA or amide linkages, and ON19^{2′OMe/8LNA/17PS} with LNA sugars but no amide linkages to determine if the enhanced duplex stability of LNA was responsible for any observed increase in activity. A scrambled control (ON31^{2′OMe/17PS scrambled}, FIG. 6) with a 2′OMe/PS backbone was also included in the study to rule out off target effects leading to activity.

To compare the biological activity independent of cell uptake, Lipofectamine 2000 (LF2000), a cationic liposome transfection/delivery reagent, was used. All three target-complementary PS-ONs were active in the assay (ON20^{2′OMe/17PS}, ON18^{2′OMe/4LAL/13PS}, and ON19^{2′OMe/8LNA/17PS}), whereas the PO-ONs (ON14^{DNA/4LAL/13PO}, ON16^{2′OMe/4LAL/13PO} and ON17^{2′OMe/17PO}) were all inactive at 100 nM (FIG. 6a). These results indicate that phosphorothioate modification in a fully ‘target-complementary’ sequence is necessary for splice-switching activity. This agrees with previous studies and could result from the PS group leading to nuclear enrichment⁵⁷ of the oligonucleotides, and/or recruiting ILF2/3 to the RNA transcript⁵⁸. Notably, the addition of amide bonds flanked with LNA significantly improved the splice-switching activity of 2′OMe/PS ONs at the lower concentrations (6.25 nM and 12.5 nM), probably as a result of improved target affinity (FIG. 6b).

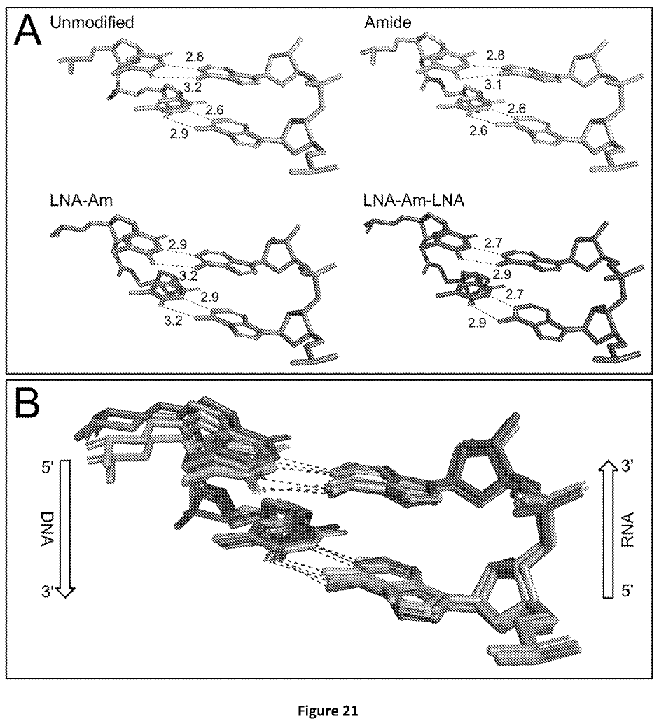

Next, we compared the naked (gymnotic) uptake of the ONs. These conditions more closely represent in vivo applications where transfection agents such as LF2000 cannot be used. We seeded cells at low confluency, added the oligonucleotides in fresh media after 16 h, and measured luciferase activity after a further 96 h. The presence of just four amide bonds flanked both sides by LNA (ON18^{2′OMe/4LAL/13PS}) significantly increased the activity in a dose-dependent manner when compared with ON20^{2′OMe/17PS} (FIG. 6c). Greater than 5-fold increase in activity was observed for gymnotic delivery which compared to a maximum of 3-fold increase in activity for the LF2000-mediated transfection. This suggests that synergy between the PS and LNA-amide modifications leads to enhanced productive delivery into cells. These results confirm our hypothesis that the combination of PS, amide, and LNA would result in ONs with improved therapeutic properties, possibly due to a combination of reduced charge from the amides and interactions with the PS backbone. Interestingly, ON19^{2′OMe/8LNA/PS} with LNA and no amides showed only slight dose response in activity, even at the highest concentration tested (FIG. 6c, FIG. 23). This could be due to binding to off-targets, altered rigidity, or undesirable secondary structures induced by extreme stability caused by the LNA sugars, reducing the ability of the ON to interact with the cell surface, a mechanism for productive uptake. ON16^{2′OMe/4LAL/13PO} with LNA-amides and no PS linkages also displayed slight gymnotic splice-switching activity (FIG. 23).

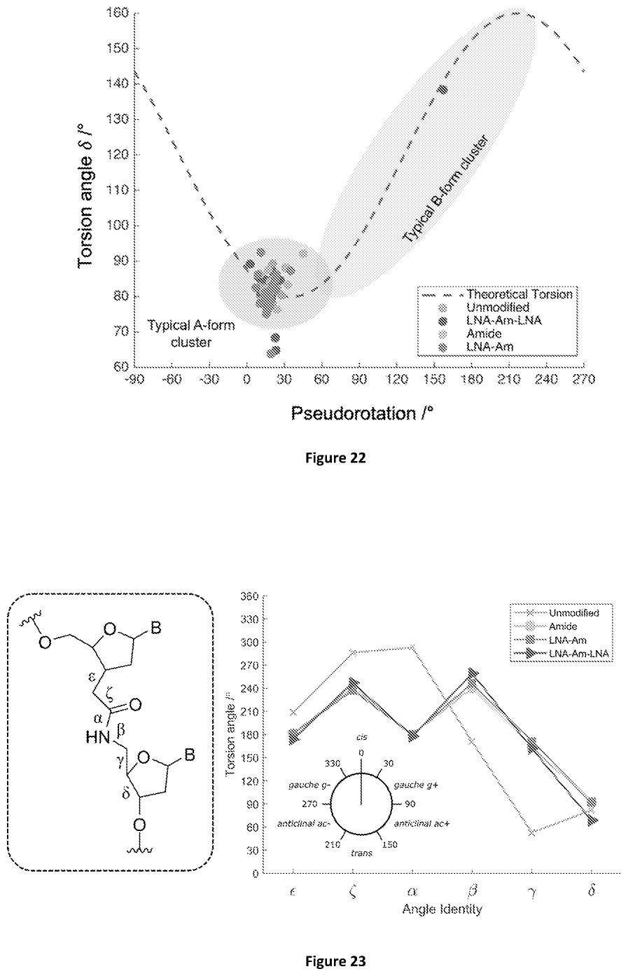

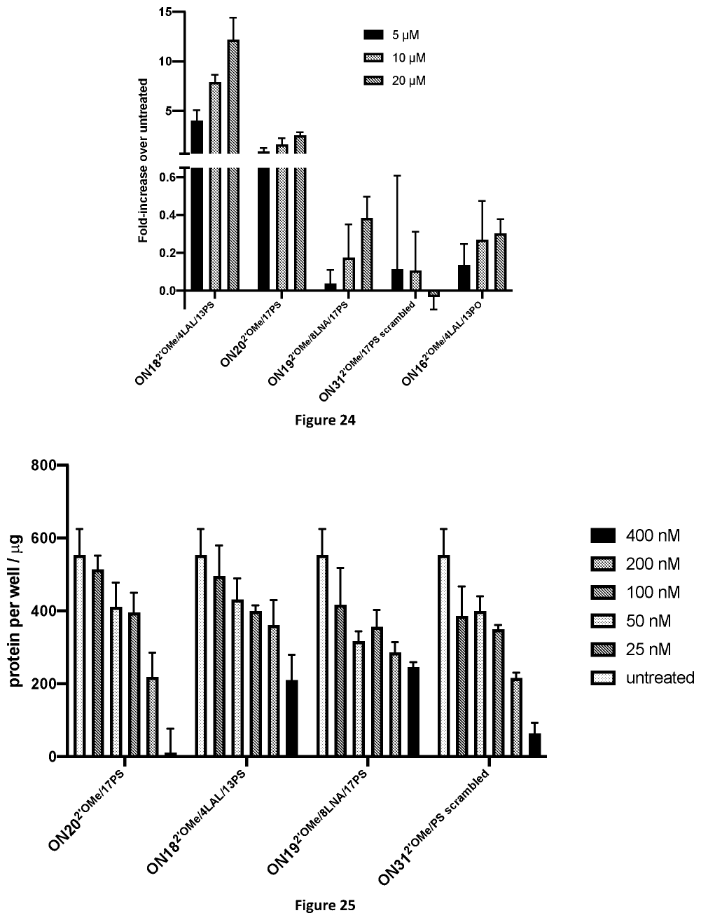

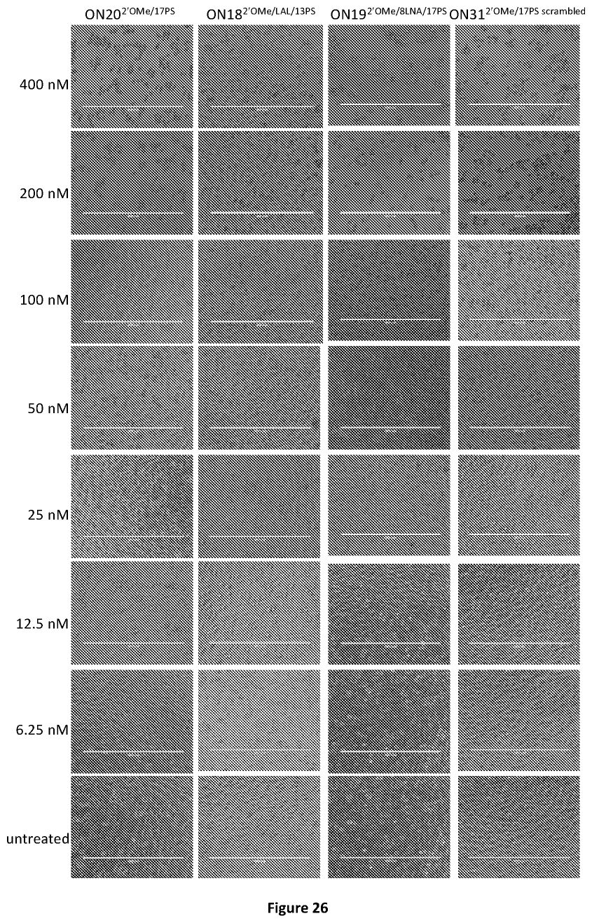

We compared the viability of the HeLa cells following lipofection using a WST-1 cell proliferation assay (FIG. 6d). At the highest concentration tested (400 nM) the cells treated with ON20^{2′OMe/17PS} were only 21% viable, whereas the cells treated with the same concentration of ON18^{2′OMe/4LAL/13PS} were 50% viable, demonstrating that the LNA-amide linkage significantly reduces the cytotoxicity of ONs delivered with LF2000. This is verified by analysis of the protein levels (FIG. 24) and visible cell death (FIG. 6e, and FIG. 25). It supports the use of the combination of LNA-amide, 2′OMe and PS modifications for in-vitro studies using transfection agents such as LF2000. Interestingly, the oligonucleotide containing 8 LNA sugars without amide linkages (ON19^{2′OMe/8LNA/17PS}) had a poor toxicity profile. This could be due to off-target effects and could also explain why, despite showing the highest affinity towards RNA in the UV melting studies, the LNA modified ON19^{2′OMe/LNA/17PS} was not most active in the exon-skipping assay. Further detailed studies are required to determine the therapeutic relevance of the above findings on toxicity.

Given that cell uptake remains a major challenge when developing new therapeutic oligonucleotides, the results shown in FIG. 6 highlight the potential of our modification strategy; the synergy of LNA-flanked amide linkages and phosphorothioate modifications result in an oligonucleotide with superior biological activity.

### CONCLUSIONS

We have developed methodology to synthesise LNA monomers with 3′-ethanoic acid and 5-amino groups and incorporate them into oligonucleotides that combine the favourable properties of LNA sugars, amide backbones, and phosphorothioate groups synergistically. The methodology is high yielding and has the potential to be automated, an important consideration for therapeutic oligonucleotide development. The resulting constructs have remarkable resistance to enzymatic degradation, and bind to complementary RNA with affinity and selectivity superior to unmodified ONs, but crucially not as tightly as LNA. X-ray crystallography revealed that the artificial backbone causes minimal structural deviation in DNA:RNA hybrids, consistent with the excellent affinity of the modified ONs for complementary RNA. Oligonucleotides with alternating LNA-amide and phosphodiester (or phosphorothioate) backbones cannot give rise to LNA mononucleotides (modified dNTPs) in the presence of cellular nucleases, and their favourable toxicity profile relative to LNA may reflect this. Cell studies with gymnotic delivery revealed that the substitution of just four LNA-flanked amides in a 2′OMe phosphorothioate background significantly improves naked (gymnotic) uptake. Poor cellular uptake is currently a major barrier in oligonucleotide technologies in general, and we propose that combining the PS and LNA modifications with charge-neutral amide backbones such as AM1 could lead to more potent oligonucleotides for clinical applications.

### Supplementary Information and Experimental Section

### Small Molecule Synthesis

### General

Unless otherwise stated, reactions were performed in oven-dried glassware under an inert argon using anhydrous solvents. Anhydrous solvents were collected from an mBraun SPS-800 bench top solvent purification system, having passed through anhydrous alumina columns. Solvents for phosphitylation reaction were degassed by bubbling with argon before used and pyridine and CH₂Cl₂ were further purified by distillation over KOH or CaH respectively. Anhydrous dichloroethane (Aldrich) was used as supplied without further purification. 3-O-Benzyl 4-C-(methanesulfonyloxymethyl)-5-O-methanesulfonyl-1,2-O-isopropylidene-a-D-ribofuranose was purchased from Carbosynth. All other chemicals were used as obtained from commercial sources without further purification.

Thin layer chromatography (TLC) was performed using Merck pre-coated 0.23 mm thick plates of Kieselgel 60 F254 and visualised using UV (λ=254 nm) or by staining with KMnO₄, p-anisaldehyde, dinitrophenylhydrazine, iodine, or ninhydrin (depending on functionality). All retention factors (R_{f}) are given to 0.01 with the solvent system reported in parentheses. Column chromatography was carried out using Geduran Silica Gel 60 from Merck.

Melting points (mp) were measured using Gallenkamp melting point apparatus and are uncorrected.

¹H, ¹³C and ³¹P NMR spectra were recorded on a BrukerAVIIIHD 400, BrukerAVII 500 (with a ¹³C cryoprobe), or Bruker NEO 600 (with broadband helium cryoprobe) spectrometer operating at 400, 500 or 600 MHz respectively using an internal deuterium lock at ambient probe temperatures. ¹H NMR chemical shifts (6) are quoted to the nearest 0.01 ppm and are referenced relative to residual solvent peak. Coupling constants (J) are given to the nearest 0.1 Hz. The following abbreviations are used to indicate the multiplicity of signals: s=singlet, d=doublet, t=triplet, q=quartet, m=multiplet, and br=broad. Data is reported as follows: chemical shift (multiplicity, coupling constant(s), integration). ¹³C NMR chemical shifts (6) are quoted to the nearest 0.1 ppm and are reference relative to the deuterated solvent peak. NMR assignments are supported by DEPT, COSY, HMQC, and HMBC where necessary.

High resolution mass spectra (HRMS) were recorded on a Thermo Scientific Exactive Mass Spectrometer equipped with a Waters Equity autosampler and pump by the University of Oxford Chemistry Departmental Mass Spectrometry Service, and reported mass values are within ±5 ppm mass units unless otherwise stated.

Unless otherwise stated, yields refer to analytically pure compounds.

### 4-C-(Methanesulfonyloxymethyl)-5-O-methanesulfonyl-1,2-O-isopropylidene-α-D-ribofuranose 2

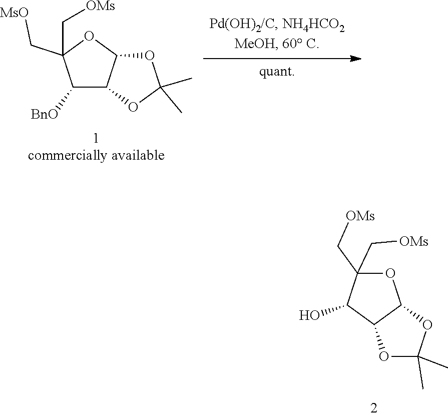 [SMILES: CC1(C)O[C@H]2OC(COS(C)(=O)=O)(COS(C)(=O)=O)[C@@H](OCc3ccccc3)[C@H]2O1 ; CC1(C)O[C@H]2OC(COS(C)(=O)=O)(COS(C)(=O)=O)[C@@H](O)[C@H]2O1]

Commercially available 3-O-benzyl 4-C-(methanesulfonyloxymethyl)-5-O-methanesulfonyl-1,2-O-isopropylidene-a-D-ribofuranose 1 (9.8 g, 21.1 mmol) and ammonium formate (10 g, 159 mmol, 7.5 eq) were dissolved in MeOH (250 mL) and 20 wt % palladium hydroxide on carbon (1.48 g, 2.11 mmol, 10 mol %) was added. The flask was flushed with argon and the reaction was stirred at 60° C. overnight. A large volume of gas is generated within the first hour of the reaction presenting a risk of over-pressurisation. The reaction was filtered through celite to remove the catalyst and the solvent was removed under vacuum. The resulting solid was dissolved in EtOAc (100 mL), washed with a half-saturated aqueous solution of NaCl (2×100 mL), dried over MgSO₄, and evaporated to dryness to give 2 (7.9 g, 21.0 mmol) as a white solid in quantitative yield.

TLC (EtOAc:40-60 petroleum ether (PE), 7:3 v/v) R_{f}: 0.36;

¹H NMR (400 MHz, CDCl₃): δ 5.87 (d, J=4.0 Hz, 1H), 4.75 (dd, J=6.1, 4.0 Hz, 1H), 4.65 (d, J=11.6 Hz, 1H), 4.50 (d, J=11.6 Hz, 1H), 4.39 (dd, J=7.7, 6.1 Hz, 1H), 4.33 (d, J=10.9 Hz, 1H), 4.29 (d, J=10.9 Hz, 1H), 3.12 (s, 3H), 3.08 (s, 3H), 2.88 (d, J=7.7 Hz, 1H), 1.67-1.66 (s, 3H), 1.38 (s, 3H);

¹³C NMR (101 MHz, CDCl₃): δ 114.1, 104.7, 84.3, 79.2, 72.7, 69.3, 68.6, 38.0, 37.7, 26.2, 26.0; HRMS (m/z): [M+Na]⁺ calcd. for C₁₁H₂₀O₁₀NaS₂⁺, 399.0390; found, 399.0389.

### 4-C-(Methanesulfonyloxymethyl)-5-O-methanesulfonyl-1,2-O-isopropylidene-α-D-erythro-pentofuranos-3-ulose 3

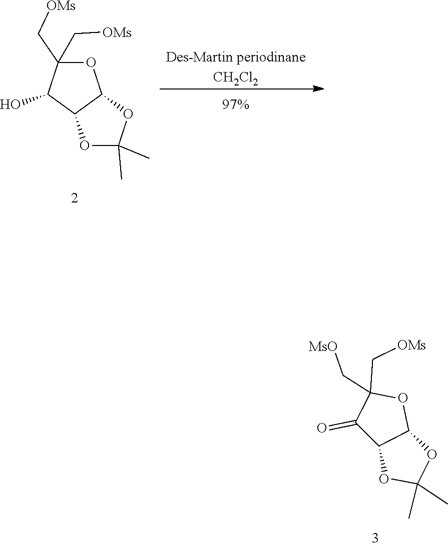 [SMILES: CC1(C)O[C@H]2OC(COS(C)(=O)=O)(COS(C)(=O)=O)[C@@H](O)[C@H]2O1 ; CC1(C)O[C@H]2OC(COS(C)(=O)=O)(COS(C)(=O)=O)C(=O)[C@H]2O1]

Alcohol 2 (17.4 g, 46.3 mmol) and Dess-Martin periodinane (29.7 g, 70.0 mmol, 1.5 eq) were dissolved in CH₂Cl₂ (300 mL) and the reaction was stirred at room temperature overnight. A solution of Na₂S₂O₃ in saturated aqueous NaHCO₃ (10% w/v, 200 mL) was added slowly to the reaction and stirring was continued until bubbling ceased. The biphasic mixture was filtered through celite to remove the white precipitate and the organic layer collected. The organic layer was washed twice more with saturated NaHCO₃ (200 mL), dried over Na₂SO₄, and evaporated to dryness to give 3 (16.7 g, 44.7 mmol) as colourless oil in 97% yield which crystallised on standing. This was used without further purification

TLC (EtOAc:40-60 PE, 6:4 v/v) R_{f}: 0.24;

¹H NMR (400 MHz, CDCl₃): δ 6.16 (d, J=4.1 Hz, 1H), 4.52 (d, J=4.1 Hz, 1H), 4.46 (d, J=11.0 Hz, 1H), 4.45 (d, J=11.7 Hz, 1H), 4.33 (d, J=11.7 Hz, 1H), 4.32 (d, J=11.0 Hz, 1H), 3.11 (s, 3H), 3.03 (s, 3H), 1.56 (s, 3H), 1.39 (s, 3H);

¹³C NMR (101 MHz, CDCl₃): δ 205.0, 115.6, 103.0, 84.3, 76.7, 69.5, 68.9, 38.2, 37.9, 27.2, 26.8; HRMS (m/z): [M+Na]⁺ calcd. for C₁₁H₁₈O₁₀NaS₂⁺, 397.0234; found, 397.0231.

### Ethyl 2-((3aR,6aR)-2,2-dimethyl-5,5-bis(((methylsulfonyl)oxy)methyl)dihydrofuro[2,3-d][1,3]dioxol-6(5H)-ylidene)acetate 4

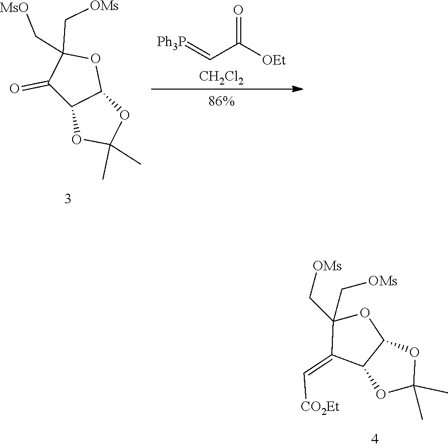

A solution of compound 3 (15.1 g, 40.4 mmol) and (carbethoxymethylene)triphenylphosphorane (16.9 g, 48.5 mmol, 1.2 eq) in CH₂Cl₂ (80 mL) was stirred at room temperature overnight. After removal of the solvent the resulting orange gum was triturated with EtOH, forming a white precipitate. The precipitate was collected by filtration, washed with cold EtOH, and dried under vacuum. The crude product was then purified by recrystallisation from hot EtOH to yield alkene 4 (15.4 g, 33.1 mmol) in 86% yield.

TLC (EtOAc:40-60 PE, 6:4 v/v) R_{f}: 0.64;

mp: 88-93° C. (crystallised from EtOH);

¹H NMR (400 MHz, CDCl₃): δ 6.07 (d, J=1.3 Hz, 1H), 5.93 (d, J=3.7 Hz, 1H), 5.79 (dd, J=3.7, 1.3 Hz, 1H), 4.53-4.19 (m, 6H), 3.11 (s, 3H), 3.07 (s, 3H), 1.59 (s, 3H+H₂O), 1.41 (s, 3H), 1.32 (t, J=7.1 Hz, 3H);

¹³C NMR (101 MHz, CDCl₃): δ 164.0, 151.2, 121.4, 114.5, 105.6, 85.0, 78.7, 70.1, 69.5, 61.5, 38.2, 37.9, 27.2, 26.4, 14.2;

HRMS (m/z): [M+Na]⁺ calcd. for C₁₅H₂₄O₁₁NaS₂⁺, 467.0652; found, 467.0652. Ethyl 2-((3aR,6S,6aR)-2,2-dimethyl-5,5-bis(((methylsulfonyl)oxy)methyl)tetrahydrofuro[2,3-d][1,3]dioxol-6-yl)acetate 5

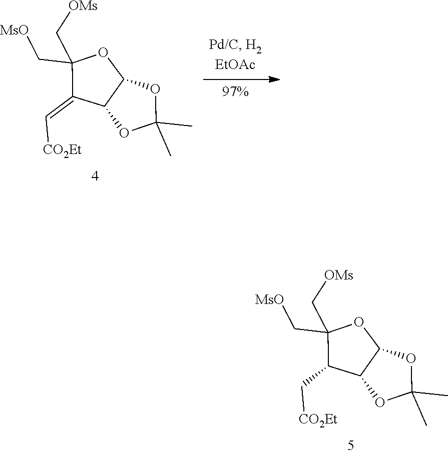 [SMILES: CCOC(=O)/C=C1\[C@H]2OC(C)(C)O[C@H]2OC1(COS(C)(=O)=O)COS(C)(=O)=O ; CCOC(=O)C[C@H]1[C@H]2OC(C)(C)O[C@H]2OC1(COS(C)(=O)=O)COS(C)(=O)=O]

A solution of 4 (13.0 g, 29.3 mmol) in EtOAc (300 mL) was placed under an atmosphere of argon before 5% palladium on activated carbon (1.3 g, 1.5 mmol, 5 mol %) was added. The flask was evacuated under vacuum and refilled with H₂ gas three times to ensure a hydrogen atmosphere and vigorously stirred overnight. The reaction was monitored by NMR. Once complete, the mixture was filtered through a pad of celite and the solvent removed under reduced pressure to yield 5 (12.7 g, 28.4 mmol) as a colourless solid in 97% yield which was used without further purification. The use of Pd/C and hydrogen gas along with flammable solvents poses a significant fire risk.

TLC (EtOAc:40-60 PE, 6:4 v/v) R_{f}: 0.64;

mp: 88-95° C. (crystallised from a mixture of MeOH and EtOH);

¹H NMR (400 MHz, CDCl₃): δ 5.85 (d, J=3.9 Hz, 1H), 4.87 (dd, J=5.2, 3.9 Hz, 1H), 4.59 (d, J=10.8 Hz, 1H), 4.31 (d, J=10.7 Hz, 1H), 4.30 (d, J=10.8 Hz, 1H), 4.23 (d, J=10.7 Hz, 1H), 4.18 (apparent dq, J=7.1, 2.2 Hz, 2H), 3.12 (s, 3H), 3.07 (s, 3H), 2.91-2.65 (m, 2H), 2.57 (dd, J=16.9, 5.5 Hz, 1H), 1.60 (s, 3H), 1.30 (s, 3H), 1.28 (t, J=7.2 Hz, 3H);

¹³C NMR (101 MHz, CDCl₃): δ 171.3, 112.9, 105.3, 84.1, 81.7, 70.1, 68.2, 61.3, 42.7, 38.1, 37.7, 28.6, 26.3, 25.5, 14.2;

HRMS (m/z): [M+Na]⁺ calcd. for C₁₅H₂₆O₁₁NaS₂⁺, 469.0809; found, 469.0811.

### (3R,4S)-4-(2-Ethoxy-2-oxoethyl)-5,5-bis(((methylsulfonyl)oxy)methyl)tetrahydrofuran-2,3-diyl diacetate 6

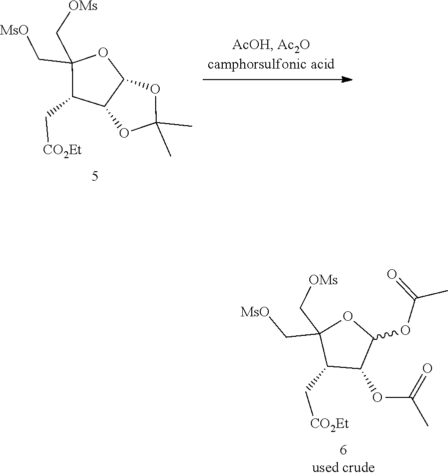 [SMILES: CCOC(=O)C[C@H]1[C@H]2OC(C)(C)O[C@H]2OC1(COS(C)(=O)=O)COS(C)(=O)=O ; CCOC(=O)C[C@H]1[C@@H](OC(C)=O)C(OC(C)=O)OC1(COS(C)(=O)=O)COS(C)(=O)=O]

To a solution of compound 5 (4.9 g, 11.0 mmol) in acetic acid (50 mL) and acetic anhydride (38 mL) was added camphorsulfonic acid (CSA) (120 mg, 0.52 mmol) and the solution was stirred at 80° C. for 90 min. A second portion of CSA (120 mg, 0.52 mmol) was added and stirring continued at 80° C. for 90 min. This was repeated twice more, and the reaction left to stir overnight at 80° C. The volatiles were removed under reduced pressure and the resulting brown gum was co-evaporated with toluene (3×50 mL), dissolved in EtOAc (150 mL), washed with a saturated aqueous solution of NaHCO₃ (5×100 mL), washed with brine (1×100 mL), dried over Na₂SO₄, and evaporated to dryness to yield crude compound 6 (5.3 g, assume quantitative) which was used without purification in the next step. We found the compound was not stable to column chromatography.

TLC (EtOAc:40-60 PE, 6:4 v/v) R_{f}: 0.38;

¹H NMR (400 MHz, CDCl₃): δ 6.10 (s, 1H), 5.32 (d, J=5.0 Hz, 1H), 4.40 (d, J=3.3 Hz, 2H), 4.28 (s, 2H), 4.20-4.11 (m, 2H), 3.08 (s, 3H), 3.07 (s, 3H), 3.03 (dd, J=7.7, 4.9 Hz, 1H), 2.62 (t, J=7.5 Hz, 2H), 2.14 (s, 3H), 2.11 (s, 3H), 1.26 (t, J=7.2 Hz, 3H);

¹³C NMR (101 MHz, CDCl₃): δ 170.9, 169.5, 169.3, 97.8, 84.6, 77.8, 71.4, 66.9, 61.6, 40.4, 38.0, 37.6, 28.6, 21.1, 20.8, 14.2;

HRMS (m/z): [M+Na]⁺ calcd. for C₁₆H₂₆O₁₃NaS₂⁺, 513.0707; found, 513.0706.

### Thymine LNA Acid Precursor 7a

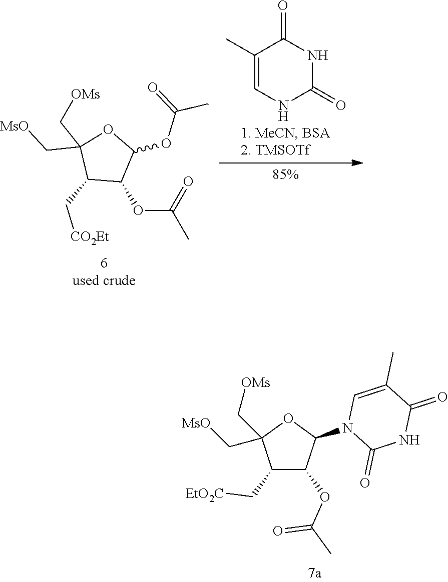

Crude compound 6 (2.5 g, 5.1 mmol) and thymine (0.8 g, 6.4 mmol, 1.25 eq) were co-evaporated with anhydrous MeCN (3×15 mL). The mixture was then dissolved in anhydrous MeCN (12.5 mL) and bis(trimethylsilyl)acetamide (BSA) (3.5 mL, 14.2 mmol, 2.8 eq) was added. The solution was heated to reflux for 1 h. The reaction was cooled to room temperature and TMSOTf (1.25 mL, 6.8 mmol, 1.4 eq) was added. The reaction was then heated to reflux overnight resulting in a dark red solution. The reaction was cooled to room temperature, diluted with CH₂Cl₂ (12.5 mL), and a half saturated aqueous solution of NaHCO₃ (25 mL) was added with stirring (bubbles are generated). The organic layer became a pale-yellow colour and was subsequently washed with saturated aqueous NaHCO₃ (25 mL) and brine (25 mL). The organic phase was dried over Na₂SO₄ and evaporated to dryness. The crude brown foam was purified by column chromatography (0-7% MeOH in CH₂Cl₂) to give compound 7a (2.42 g, 4.4 mmol) as a beige foam in 85% yield.

TLC (CH₂Cl₂:MeOH, 17:1 v/v) R_{f}: 0.55;

¹H NMR (400 MHz, CDCl₃): δ 9.29 (s, 1H), 7.03 (d, J=1.2, 1H), 5.58 (dd, J=7.6, 1.7, 1H), 5.49 (d, J=1.7, 1H), 4.47 (s, 2H), 4.38 (d, J=11.0, 1H), 4.32 (d, J=11.0, 1H), 4.15 (q, J=7.1, 2H), 3.55 (ddd, J=9.6, 7.6, 6.2, 1H), 3.10 (s, 3H), 3.09 (s, 3H), 2.65 (dd, J=16.8, 9.6, 1H), 2.57 (dd, J=16.8, 6.2, 1H), 2.14 (s, 3H), 1.91 (d, J=1.2, 3H), 1.25 (t, J=7.1, 3H);

¹³C NMR (101 MHz, CDCl₃): δ 170.8, 170.3, 163.9, 150.3, 138.5, 111.6, 95.8, 85.5, 78.5, 70.1, 67.4, 61.5, 41.4, 37.9, 37.6, 29.1, 20.8, 14.3, 12.4;

HRMS (m/z): [M+Na]⁺ calcd. for C₁₉H₂₈N₂O₁₃NaS₂⁺, 579.0925; found 579.0924.

### N⁴-Benzoylcytosine LNA acid precursor 7b

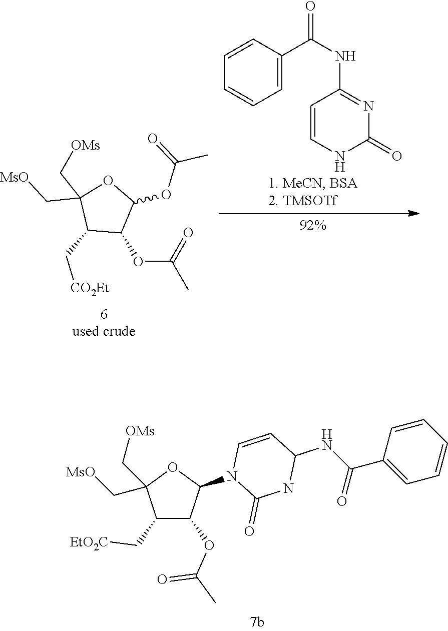 [SMILES: CCOC(=O)C[C@H]1[C@@H](OC(C)=O)C(OC(C)=O)OC1(COS(C)(=O)=O)COS(C)(=O)=O ; O=C(Nc1cc[nH]c(=O)n1)c1ccccc1 ; CCOC(=O)C[C@H]1[C@@H](OC(C)=O)[C@H](N2C=CC(NC(=O)c3ccccc3)NC2=O)OC1(COS(C)(=O)=O)COS(C)(=O)=O]

Compound 6 (1.04 g, 2.1 mmol) and N⁴-benzoylcytosine (0.912 g, 4.0 mmol, 2.0 eq) were co-evaporated with anhydrous MeCN (3×15 mL). The mixture was then dissolved in anhydrous MeCN (12.5 mL) and BSA (1.0 mL, 4.1 mmol, 1.9 eq) was added. The suspension was heated to reflux for 1 h. The reaction was cooled to room temperature and TMSOTf (0.45 mL, 2.5 mmol, 1.2 eq) was added. The reaction was then heated to reflux overnight resulting in a dark red solution. The reaction was cooled to room temperature, diluted with CH₂Cl₂ (12.5 mL), and a half saturated aqueous solution of NaHCO₃ was added with stirring. The organic layer was subsequently washed with saturated aqueous NaHCO₃ (25 mL) and brine (25 mL). The organic phase was dried over Na₂SO₄ and evaporated to dryness. The crude brown foam was purified by column chromatography (50-100% EtOAc in 40-60 PE) to give 7b (1.25 g, 1.9 mmol) as a pale-yellow foam in 92% yield.

TLC (CH₂Cl₂:MeOH, 17:1 v/v) R_{f}: 0.53;

¹H NMR (400 MHz, CDCl₃): δ 8.97 (s, 1H), 7.93 (d, J=7.7, 2H), 7.73 (d, J=7.5, 1H), 7.65-7.50 (m, 4H), 5.67 (dd, J=7.4, 1.5, 1H), 5.58 (d, J=1.5, 1H), 4.62-4.49 (m, 2H), 4.45-4.36 (m, 2H), 4.16 (q, J=7.1, 2H), 3.66 (apparent dt, J=9.1, 7.0, 1H), 3.11 (s, 3H), 3.09 (s, 3H), 2.69 (dd, J=16.8, 9.1, 1H), 2.59 (dd, J=16.7, 6.8, 1H), 2.17 (s, 3H), 1.26 (t, J=7.1, 3H);

¹³C NMR (151 MHz, CDCl₃): δ 170.7, 170.4, 166.3 (broad due to rotamers), 162.6, 153.3 (broad due to rotamers), 148.5, 133.8, 132.4, 129.3, 128.2, 97.8, 96.9 (broad due to rotamers), 86.7, 78.7, 70.1, 67.6, 61.5, 41.4, 38.0, 37.7, 29.3, 20.8, 14.3;

HRMS (m/z): [M+H]1 calcd. for C₂₅H₃₂O₁₃N₃S₂⁺, 646.1371; found, 646.1367.

### N⁴-Benzoyl methylcytosine LNA precursor 7c

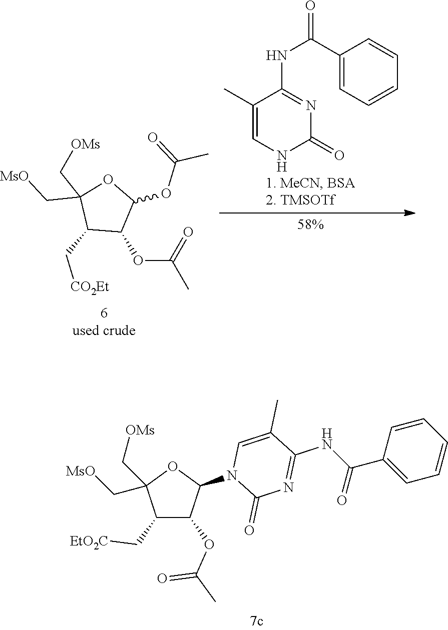 [SMILES: CCOC(=O)C[C@H]1[C@@H](OC(C)=O)C(OC(C)=O)OC1(COS(C)(=O)=O)COS(C)(=O)=O ; Cc1c[nH]c(=O)nc1NC(=O)c1ccccc1 ; CCOC(=O)C[C@H]1[C@@H](OC(C)=O)[C@H](n2cc(C)c(NC(=O)c3ccccc3)nc2=O)OC1(COS(C)(=O)=O)COS(C)(=O)=O]

A suspension of N⁴-benzoyl methylcytosine (808 mg, 3.5 mmol, 1.5 eq), compound 6 (1.13 g, 2.3 mmol) and BSA (1.5 mL, 6.1 mmol, 2.7 eq) in anhydrous MeCN (13.5 mL) was heated to reflux for 1 h. The solution was cooled to room temperature, TMSOTf (0.5 mL, 2.8 mmol, 1.2 eq) was added dropwise with stirring and the reaction was then heated to reflux overnight. After cooling to room temperature, the reaction was diluted with EtOAc (30 mL) and a saturated aqueous solution of NaHCO₃ (30 mL) was added slowly (generates bubbles). Stirring the biphasic mixture generated a precipitate, which was filtered removing the precipitate prior to workup. The organic layer was collected, washed with saturated aqueous NaHCO₃ (2×30 mL) followed by brine (30 mL), dried over MgSO₄, and evaporated to dryness to give an orange foam. This was purified by column chromatography (0-10% MeOH in CH₂Cl₂) to give 7c (885 mg, 1.3 mmol) as a beige foam in 58% yield.

TLC (CH₂Cl₂:MeOH, 17:1 v/v) R_{f}: 0.5;

¹H NMR (400 MHz, CDCl₃): δ 13.24 (s, 1H), 8.34-8.29 (m, 2H), 7.54 (tt, J=7.4, 1.4, 1H), 7.45 (t, J=7.4, 2H), 7.21 (d, J=1.2, 1H), 5.62 (dd, J=7.5, 1.7, 1H), 5.54 (d, J=1.7, 1H), 4.49 (d, J=10.8, 2H), 4.46 (d, J=10.8, 1H), 4.41 (d, J=11.0, 1H), 4.33 (d, J=11.0, 1H), 4.17 (q, J=7.1, 2H), 3.58 (apparent dt, J=9.2, 6.9, 1H), 3.11 (s, 3H), 3.09 (s, 3H), 2.67 (dd, J=16.8, 9.2, 1H), 2.58 (dd, J=16.8, 6.6, 1H), 2.17 (s, 3H), 2.12 (d, J=1.2, 3H), 1.27 (t, J=7.1, 3H);

¹³C NMR (151 MHz, CDCl₃): δ 180.0, 170.7, 170.2, 159.7, 147.8, 139.1, 137.1, 132.8, 130.2, 128.3, 112.7, 96.1, 85.7, 78.5, 69.8, 67.3, 61.5, 41.3, 38.0, 37.7, 29.1, 20.8, 14.3, 13.5;

HRMS (m/z): [M+H]⁺ calcd. for C₂₆H₃₄O₁₃N₃S₂⁺, 660.1528; found, 660.1522.

### N⁶-Benzoyladenine LNA acid precursor 7d

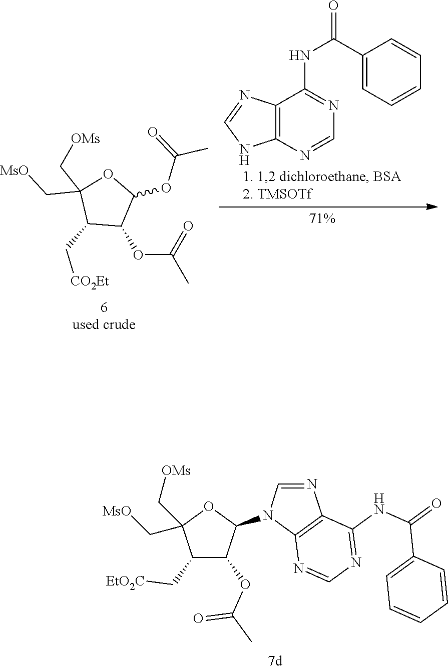

N⁶-Benzoyladenine (1.15 g, 4.8 mmol) and compound 6 (2.63 g, 5.3 mmol, 1.1 eq) were suspended in anhydrous 1,2-dichlorethane (22 mL) and BSA (3.13 mL, 12.8 mmol, 2.7 eq) was added. The solution was heated to reflux for 1 h. The reaction was cooled to room temperature and TMSOTf (2.0 mL, 11 mmol, 2.3 eq) was added. The reaction was then heated to reflux overnight resulting in a dark red solution. The reaction was cooled to room temperature, diluted with CH₂Cl₂ (12.5 mL), and added to a saturated aqueous solution of NaHCO₃ (22 mL) slowly with stirring (bubbles are generated). The organic layer was subsequently washed with saturated aqueous NaHCO₃ (25 mL) and brine (25 mL), dried over Na₂SO₄, and evaporated to dryness. The crude brown foam was purified by silica column chromatography (0-100% EtOAc in 40-60 PE) to give compound 7d (2.29 g, 3.4 mmol) as a beige foam in 71% yield.

TLC (EtOAc) R_{f}: 0.35;

¹H NMR (400 MHz, CDCl₃): δ 9.17 (s, 1H), 8.80 (s, 1H), 8.12 (s, 1H), 8.04-7.93 (m, 2H), 7.64-7.55 (m, 1H), 7.51 (t, J=7.51, 2H), 6.13 (d, J=1.1, 1H), 5.88 (dd, J=6.7, 1.1, 1H), 4.55 (d, J=1.9, 2H), 4.48 (d, J=10.9, 1H), 4.40 (d, J=10.9, 1H), 4.16 (q, J=7.1, 2H), 4.12-4.02 (m, 1H), 3.11 (s, 3H), 2.95 (s, 3H), 2.72 (apparent dd, J=7.8, 4.9, 2H), 2.19 (s, 3H), 1.25 (t, J=7.1, 3H);

¹³C NMR (101 MHz, CDCl₃): δ 170.7, 170.1, 164.8, 152.8, 151.1, 150.0, 142.6, 133.7, 133.0, 129.0, 128.0, 123.7, 90.8, 86.0, 79.0, 69.6, 67.0, 61.5, 41.6, 37.9, 37.6, 28.9, 20.8, 14.2;

HRMS (m/z): [M+H]1 calcd. for C₂₆H₃₂O₁₂N₅S₂, 670.1483; found, 670.1482.

### N²-Isobutyrylquanine LNA acid precursor 7e

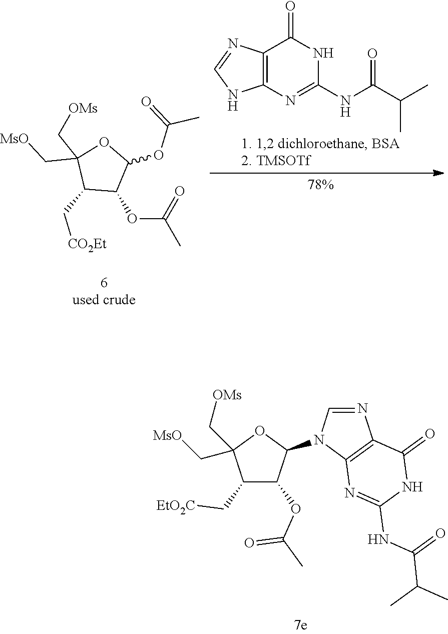

Compound 6 (2.6 g, 5.3 mmol) and N²-isobutyrylguanine (1.34 g, 6.1 mmol, 1.1 eq) were suspended in anhydrous 1,2 dichloroethane (22 mL) and BSA (3.1 mL, 12.5 mmol, 2.4 eq) was added. The suspension was heated to reflux for 1.5 h. The reaction was cooled to room temperature and TMSOTf (2.0 mL, 11 mmol, 2.1 eq) was added. The reaction was then heated to reflux for 2 h. The reaction was cooled to room temperature and added to a stirring solution of saturated aqueous NaHCO₃ (22 mL). The organic layer was subsequently washed with saturated aqueous NaHCO₃ (25 mL) and brine (25 mL). The organic phase was dried over Na₂SO₄ and evaporated to dryness. Purification by column chromatography (0-10% MeOH in EtOAc) gave 7e (2.64, 4.1 mmol) as a pale-yellow foam in 78% yield.

TLC (EtOAc:MeOH, 9:1 v/v) R_{f}: 0.45;

¹H NMR (400 MHz, CDCl₃): δ 12.16 (s, 1H), 9.44 (s, 1H), 7.75 (s, 1H), 5.96 (d, J=1.1, 1H), 5.72 (dd, J=6.5, 1.1, 1H), 4.72 (d, J=10.5, 1H), 4.49 (d, J=11.1, 1H), 4.42 (d, J=10.5, 1H), 4.32 (d, J=11.1, 1H), 4.25 (dt, J=8.9, 6.6, 1H), 4.13 (apparent qd, J=7.2, 1.1, 2H), 3.12 (s, 3H), 3.06 (s, 3H), 2.77-2.57 (m, 3H), 2.16 (s, 3H), 1.30-1.16 (m, 9H);

¹³C (101 MHz, CDCl₃): δ 179.4, 171.1, 170.0, 155.5, 148.1, 147.4, 139.2, 122.0, 91.0, 85.4, 78.5, 69.6, 67.6, 61.5, 41.4, 38.0, 37.7, 36.4, 28.6, 20.7, 19.0, 18.9, 14.2; HRMS (m/z): [M+H]1 calcd. for C₂₃H₃₄O₁₃N₅S₂⁺, 652.1589; found, 652.1586.

### Thymine LNA acid 8a

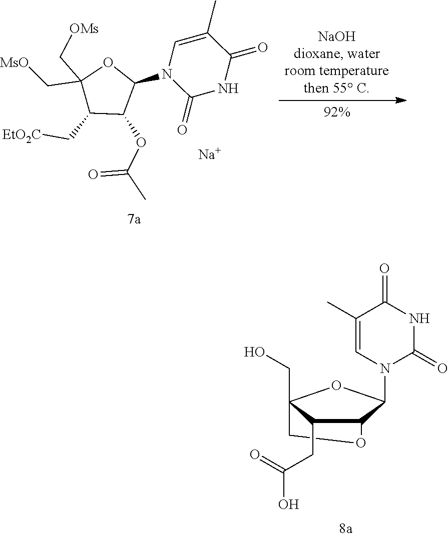 [SMILES: CCOC(=O)C[C@H]1[C@@H](OC(C)=O)[C@H](n2cc(C)c(=O)[nH]c2=O)OC1(COS(C)(=O)=O)COS(C)(=O)=O ; [Na+] ; Cc1cn([C@@H]2O[C@@]3(CO)COC2[C@H]3CC(=O)O)c(=O)[nH]c1=O]

Compound 7a (1.0 g, 1.8 mmol) was dissolved in 1,4-dioxane (4.5 mL) and water (4.5 mL) and 2 M NaOH in water (9 mL, 18 mmol, 10 eq) was added. The reaction was stirred at room temperature for 2 h until the locking step and ester hydrolysis was complete (reaction progress monitored using LCMS). The reaction was then heated to 55° C. for 1 h. The reaction was evaporated to dryness and partitioned between CH₂Cl₂ (40 mL) and water (30 mL). The aqueous layer was washed with CH₂Cl₂ (3×10 mL). The aqueous phase was acidified using 1 M HCl and washed with CH₂Cl₂ (3×20 mL). The product was then extracted from the aqueous layer using 25% iPrOH in CH₂Cl₂ (4×10 mL, until no product remained in the aqueous layer as determined by TLC), dried over Na₂SO₄, and evaporated to dryness to give 8a (516 mg, 1.7 mmol) as a white solid in 92% yield which was used without further purification.

TLC (CH₂Cl₂:MeOH, 3:2 v/v+2% Et₃N) R_{f}: 0.26;

¹H NMR (400 MHz, d₆-DMSO): δ 11.39 (s, 1H), 7.62 (d, J=1.3, 1H), 5.44 (s, 1H), 4.35 (s, 1H), 3.81 (d, J=13.0, 1H), 3.77 (d, J=13.0, 1H), 3.73 (d, J=8.5, 1H), 3.60 (d, J=8.4, 1H), 3.31 (s, 1H), 2.41 (dd, J=15.5, 2.7, 1H), 2.33-2.07 (m, 2H), 1.79 (d, J=1.2, 3H);

¹³C NMR (101 MHz, d₆-DMSO): δ 173.4, 164.3, 150.5, 135.4, 108.8, 91.1, 86.8, 80.2, 71.5, 57.1, 39.7, 29.0, 12.9;

HRMS (m/z): [M−H]⁻ calcd. for C₁₃H₁₅O₇N₂⁻, 311.0885; found, 311.0882.

### N⁴-Benzoylcytosine LNA acid 8b

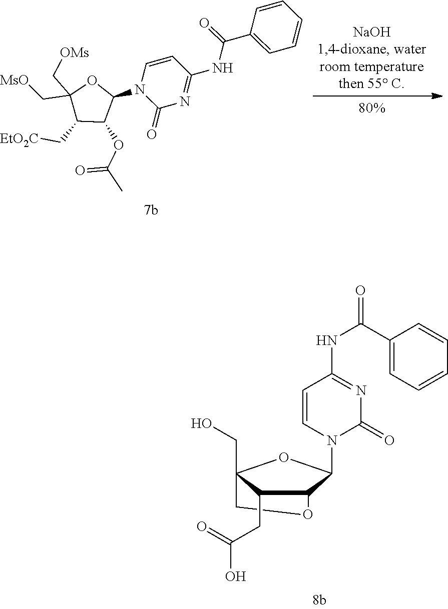 [SMILES: CCOC(=O)C[C@H]1[C@@H](OC(C)=O)[C@H](n2ccc(NC(=O)c3ccccc3)nc2=O)OC1(COS(C)(=O)=O)COS(C)(=O)=O ; O=C(O)C[C@@H]1C2OC[C@]1(CO)O[C@H]2n1ccc(NC(=O)c2ccccc2)nc1=O]

Compound 7b (400 mg, 0.62 mmol) was dissolved in 1,4-dioxane (4 mL) and 1 M NaOH in water (2 mL, 2 mmol, 3.2 eq) was added. The reaction was stirred at room temperature for 2 h until the locking step and ester hydrolysis was complete (reaction progress monitored using LCMS). The reaction was then heated to 55° C. for 2 h. The reaction was evaporated to dryness and partitioned between CH₂Cl₂ (40 mL) and water (30 mL). The aqueous phase was washed with CH₂Cl₂ (3×10 mL), acidified using 1 M HCl and further washed with CH₂Cl₂ (3×20 mL). The product was then extracted from the aqueous layer using 25% iPrOH in CH₂Cl₂ (4×10 mL, until no product remains in the aqueous layer as determined by TLC), dried over Na₂SO₄, and evaporated to dryness to give 8b (198 mg, 0.49 mmol) as a white solid in 80% yield which was used without further purification.

TLC (CH₂Cl₂:MeOH, 3:2 v/v, +2% Et₃N) R_{f}: 0.4;

¹H NMR (500 MHz, d₆-DMSO): δ 8.26 (d, J=7.5, 1H), 8.00 (dd, J=8.5, 1.3, 2H), 7.63 (tt, J=7.5, 1.3, 1H), 7.55-7.48 (m, 2H), 7.41 (d, J=7.5, 1H), 5.55 (s, 1H), 4.45 (s, 1H), 3.82 (d, J=13.1, 1H), 3.77 (d, J=13.1, 1H), 3.77 (d, J=8.4, 1H), 3.65 (d, J=8.4, 1H), 2.30 (s, 1H), 2.31-2.05 (m, 3H);

¹³C NMR (126 MHz, d₆-DMSO): δ 167.9, 167.4, 163.3, 154.0, 144.3, 133.2, 132.7, 128.5, 128.4, 95.9, 91.2, 87.4, 79.8, 71.1, 56.8, 40.1, 29.9;

HRMS (m/z): [M−H]⁻ calcd. for C₁₉H₁₈O₇N₃⁻, 400.1150; found, 400.1141.

### N⁴-Benzoyl methylcytosine LNA acid 8c

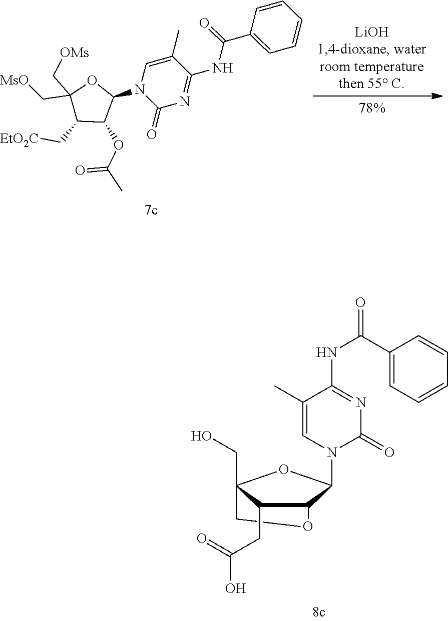 [SMILES: CCOC(=O)C[C@H]1[C@@H](OC(C)=O)[C@H](n2cc(C)c(NC(=O)c3ccccc3)nc2=O)OC1(COS(C)(=O)=O)COS(C)(=O)=O ; Cc1cn([C@@H]2O[C@@]3(CO)COC2[C@H]3CC(=O)O)c(=O)nc1NC(=O)c1ccccc1]

Compound 7c (400 mg, 0.61 mmol) was dissolved in 1,4-dioxane (4 mL) and 1 M LiOH in water (2 mL, 2 mmol, 3.3 eq) was added. The reaction was stirred at room temperature for 2 h and then heated to 55° C. After 1 h the reaction was complete as determined by LCMS. The reaction was evaporated to dryness and partitioned between CH₂Cl₂ (40 mL) and water (30 mL). The aqueous layer was washed with CH₂Cl₂ (3×40 mL), acidified with 1 M HCl, and washed once more with CH₂Cl₂ (40 mL). The product was then extracted from the aqueous layer using 15% iPrOH in CH₂Cl₂ (5×20 mL), until no product remained in the aqueous layer as determined by TLC, dried over Na₂SO₄, and evaporated to dryness to give 8c (198 mg, 0.48 mmol) as a white solid in 78% yield which was used without further purification.

TLC (CH₂Cl₂:MeOH, 3:2 v/v, +2% Et₃N) R_{f}: 0.36;

¹H NMR (500 MHz, d₆-DMSO): δ 8.23-8.16 (m, 2H), 7.95 (s, 1H), 7.60 (t, J=7.3, 1H), 7.51 (t, J=7.6, 2H), 5.53 (s, 1H), 5.25 (br s, 1H), 4.46 (s, 1H), 3.85 (d, J=13.1, 1H), 3.82 (d, J=13.1, 1H), 3.77 (d, J=8.5, 1H), 3.65 (d, J=8.5, 1H), 2.42 (dd, J=15.8, 3.2, 1H), 2.30-2.19 (m, 2H), 2.04 (s, 3H);

¹³C NMR (126 MHz, d₆-DMSO): δ 177.9, 172.9, 159.1, 147.2, 137.8, 136.5, 132.5, 129.3, 128.4, 109.1, 91.1, 87.0, 79.5, 71.1, 56.6, 39.0, 28.5, 13.4;

HRMS (m/z): [M+H]⁺ calcd. for C₂₀H₂₂O₇N₃⁺, 416.1452; found, 416.1452.

### N⁶-Benzoyladenine LNA acid 8d

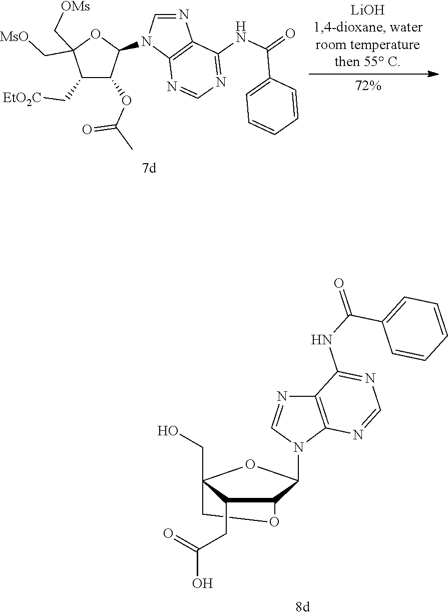 [SMILES: CCOC(=O)C[C@H]1[C@@H](OC(C)=O)[C@H](n2cnc3c(NC(=O)c4ccccc4)ncnc32)OC1(COS(C)(=O)=O)COS(C)(=O)=O ; O=C(O)C[C@@H]1C2OC[C@]1(CO)O[C@H]2n1cnc2c(NC(=O)c3ccccc3)ncnc21]

Compound 7d (500 mg, 0.75 mmol) was dissolved in 1,4-dioxane (4.8 mL) and 1 M LiOH in water (2.4 mL, 2.4 mmol, 3.2 eq). The reaction was stirred at room temperature for 2 h and then heated to 55° C. After 2 h the product formation was analysed by LCMS. The reaction was not complete and a further 0.33 eq of 1 M LiOH (266 L) was added and the reaction stirred at 55° C. for 1 h. Once complete, the reaction was evaporated to dryness and partitioned between CH₂Cl₂ (20 mL) and water (20 mL). The aqueous layer was washed with CH₂Cl₂ (3×20 mL), acidified with 1 M HCl, and washed once more with CH₂Cl₂ (20 mL). The product was then extracted from the aqueous layer using 15% iPrOH in CH₂Cl₂ (5×20 mL), dried over Na₂SO₄, and evaporated to dryness to give 8d (230 mg, 0.54 mmol) as a white solid in 72% yield which was used without further purification.

TLC (CH₂Cl₂:MeOH, 3:2 v/v, +2% Et₃N) R_{f}: 0.24;

¹H NMR (400 MHz, d₆-DMSO): δ 12.36 (s, 1H), 11.21 (s, 1H), 8.76 (s, 1H), 8.53 (s, 1H), 8.09-8.02 (m, 2H), 7.69-7.60 (m, 1H), 7.60-7.51 (m, 2H), 6.09 (s, 1H), 4.74 (s, 1H), 3.86 (m, 3H), 3.77 (d, J=8.5, 1H), 3.32 (s, 1H), 2.57 (dd, J=9.8, 4.1, 1H), 2.54-2.47 (m, 1H+solvent peak), 2.33 (dd, J=17.1, 9.8, 1H);

¹³C NMR (101 MHz, d₆-DMSO): δ 173.4, 165.8, 152.3, 151.8, 150.8, 141.5, 133.8, 132.9, 129.0, 128.9, 126.2, 90.8, 86.0, 80.5, 72.0, 57.6, 41.3, 29.1; HRMS (m/z): [M+H]⁺ calcd. for C₂₀H₂₀O₆N₅⁺, 426.1406; found, 426.1407.

### N²-Isobutyrylguanine LNA acid 8e

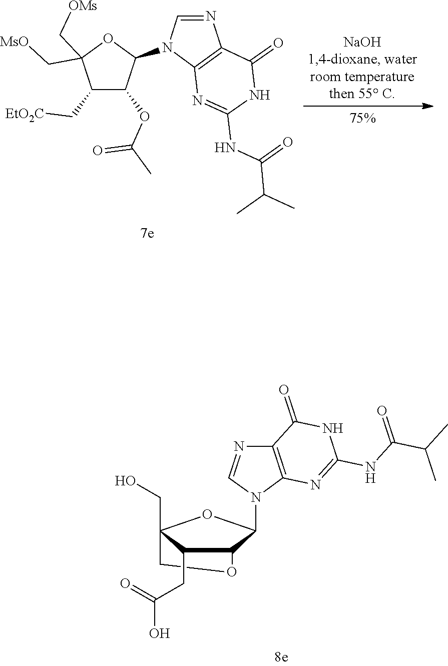 [SMILES: CCOC(=O)C[C@H]1[C@@H](OC(C)=O)[C@H](n2cnc3c(=O)[nH]c(NC(=O)C(C)C)nc32)OC1(COS(C)(=O)=O)COS(C)(=O)=O ; CC(C)C(=O)Nc1nc2c(ncn2[C@@H]2O[C@@]3(CO)COC2[C@H]3CC(=O)O)c(=O)[nH]1]

To a solution of compound 7e (105 mg, 0.17 mmol) in 1,4-dioxane (2 mL) was added 1 M NaOH in water (0.5 mL, 0.5 mmol, 3.0 eq). The reaction was stirred at room temperature for 3 h and then heated to 55° C. After 1 h the reaction was complete as determined by LCMS. The reaction was evaporated to dryness and was partitioned between CH₂Cl₂ (20 mL) and water (20 mL). The aqueous layer was washed with CH₂Cl₂ (3×20 mL), acidified with 1 M HCl, and washed once more with CH₂Cl₂ (20 mL). NaCl was added to saturate the aqueous layer and the product was extracted from the aqueous layer using 25% iPrOH in CH₂Cl₂ (5×20 mL), dried over Na₂SO₄, and evaporated to dryness to give 8e (52 mg, 0.13 mmol) as a white solid in 75% yield which was used without further purification.

TLC (CH₂Cl₂:MeOH, 3:2 v/v, +2% Et₃N) R_{f}: 0.18;

¹H NMR (400 MHz, d₆-DMSO): δ 12.14 (s, 1H), 11.80 (s, 1H), 8.14 (s, 1H), 5.82 (s, 1H), 4.61 (s, 1H), 3.82 (apparent d, J=9.8, 3H), 3.69 (d, J=8.6, 1H), 2.78 (app h, J=6.8, 1H), 2.55-2.46 (m, 2H and d₆-DMSO), 2.29 (dd, J=17.8, 10.5, 1H), 1.11 (d, J=6.8, 6H);

¹³C NMR (101 MHz, d₆-DMSO): δ 180.2, 172.9, 154.6, 148.4, 147.6, 136.1, 120.0, 90.3, 85.3, 80.1, 71.5, 57.1, 40.6, 34.7, 28.6, 18.9, 18.8;

HRMS (m/z): [M−H]⁻ calcd. for C₁₇H₂₀O₇N₅⁻, 406.1368; found, 406.1359.

### 5′-O-DMT thymine LNA acid 9a

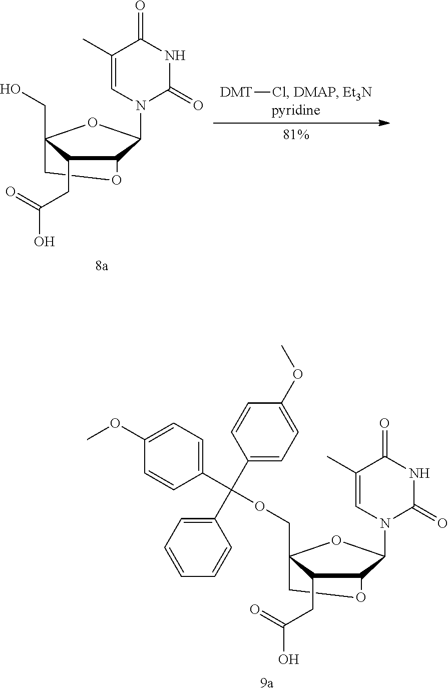

Compound 8a (400 mg, 1.28 mmol) was dissolved in pyridine (18 mL) and Et₃N (0.25 mL, 1.8 mmol, 1.1 eq) and activated 3 A molecular sieves were added. The solution was stirred at room temperature for 15 min before 4-dimethylaminopyridine (DMAP) (78 mg, 0.64 mmol, 0.5 eq) and 4,4′-dimethoxytrityl chloride (DMT-Cl) (1 g, 2.95 mmol, 2.3 eq) were added. The reaction was stirred at room temperature for 4 h before a second portion of DMT-Cl (0.8 g, 2.36 mmol, 1.8 eq) was added and the reaction was stirred at room temperature for 16 h. The molecular sieves were removed by filtration and the organic solvents were removed under vacuum. The resulting residue was purified by column chromatography (0-30% MeOH in EtOAc with a constant additive of 2% Et₃N). Following column chromatography, NMR showed significant amounts of Et₃N salts and the material was dissolved in EtOAc (25 mL) and was washed with H₂O (3×25 mL), dried over Na₂SO₄, and evaporated to dryness to yield 9a (687 mg). Rather than risk degradation, the final product was not evaporated to complete dryness and was stored and used with 0.5 eq of Et₃N present (as determined by NMR), making the final yield 81%.

TLC (CH₂Cl₂:MeOH, 4:1 v/v, +2% Et₃N) Rt: 0.18;

¹H NMR (400 MHz, DMSO-d₆): δ 11.43 (s, 1H), 7.61 (d, J=1.1, 1H), 7.49-7.37 (m, 2H), 7.37-7.21 (m, 7H), 6.91 (dd, J=9.0, 3.0, 4H), 5.48 (s, 1H), 4.42 (s, 1H), 3.74 (s, 6H), 3.64 (d, J=8.6, 1H), 3.60 (d, J=8.6, 1H), 3.50 (d, J=11.4, 1H), 3.30 (d, J=11.4, 1H underwater peak), 2.50-2.45 (m, 8H, Et₃N counterion under solvent peak), 2.42 (dd, J=9.4, 4.3, 1H), 2.17 (dd, J=16.9, 9.4, 1H), 2.00 (dd, J=16.9, 4.3, 1H), 1.59 (d, J=1.1, 3H), 0.95 (t, J=7.2, 4.5H, Et₃N);

¹³C NMR (151 MHz, d₆-DMSO): δ 172.6, 163.8, 158.2, 149.9, 144.6, 135.2, 135.0, 134.3, 129.7, 129.7, 128.0, 127.6, 126.9, 113.3, 113.3, 108.5, 89.2, 86.6, 85.8, 79.7, 71.3, 58.6, 55.0, 45.5 (Et₃N), 40.7, 28.6, 12.4, 10.7 (Et₃N);

HRMS (m/z): [M−H]⁻ calcd. for C₃₄H₃₃O₉N₂⁻, 613.2192; found, 613.2184.

### 5′-O-DMT N⁴-benzoylcytosine LNA acid 9b

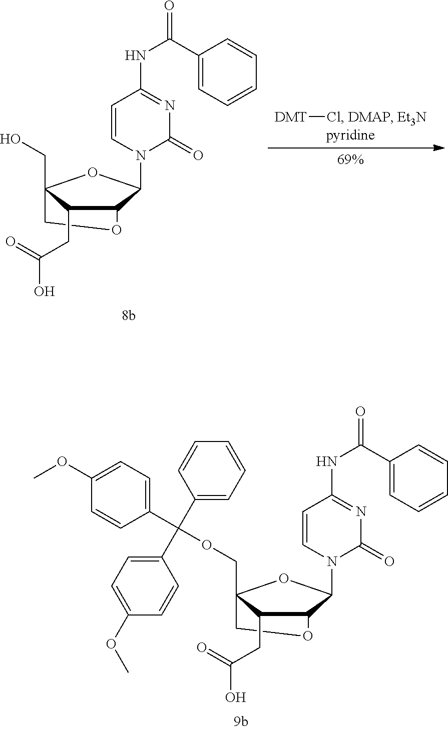

Compound 8b (100 mg, 0.25 mmol) was dissolved in pyridine (4 mL) and Et₃N (0.05 mL, 0.36 mmol, 1.4 eq) and activated 3 A molecular sieves were added. The solution was stirred at room temperature for 15 min before DMAP (16 mg, 0.13 mmol, 0.5 eq) and DMT-Cl (204 mg, 0.6 mmol, 2.5 eq) were added. The reaction was stirred at room temperature for 16 h before the molecular sieves were removed by filtration and the organic solvents removed under vacuum. The resulting residue was purified by column chromatography (0-30% MeOH in EtOAc with a constant additive of 2% pyridine) to give 9b (122 mg) in 69% yield.

TLC (CH₂Cl₂:MeOH, 3:2 v/v, +2% Et₃N) R_{f}: 0.37;

¹H NMR (400 MHz, DMSO-d₆): δ 11.17 (s, 1H), 8.33 (d, J=7.5, 1H), 8.01 (d, J=7.4, 2H), 7.64 (t, J=7.4, 1H), 7.52 (t, J=7.6, 2H), 7.46-7.22 (m, 10H), 6.94 (d, J=8.7, 4H), 5.62 (s, 1H), 4.53 (s, 1H), 3.77 (s, 6H), 3.67-3.63 (m, 2H), 3.51 (d, J=11.1, 1H), 3.41 (d, J=11.1, 1H), 2.39 (dd, J=9.3, 4.0, 1H), 2.21-2.09 (m, 1H), 1.97 (dd, J=16.6, 4.0, 1H);

¹³C NMR (151 MHz, d₆-DMSO): δ 173.2, 168.0. 163.4, 158.2, 154.0, 149.6 (pyridine), 144.4, 143.9, 135.1, 135.1, 133.2, 132.7, 129.7, 129.7, 128.5, 128.4, 128.0, 127.7, 126.9, 123.9, 113.3, 113.3, 95.8, 89.3, 87.6, 86.0, 79.3, 71.3, 58.4, 55.0, 40.1, 28.7.

HRMS (m/z): [M+H]⁺ calcd. for C₄₀H₃₈O₉N₃⁺, 704.2603; found, 704.2602.

### 5′-O-DMT N⁴-benzoyl methylcytosine LNA acid 9c

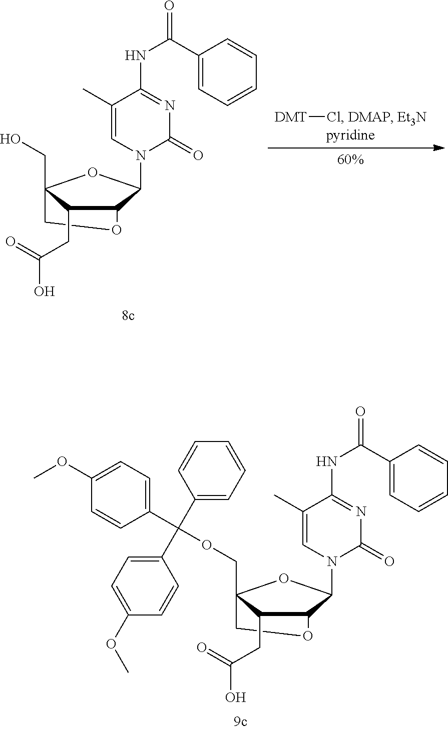

Compound 8c (100 mg, 0.24 mmol) was dissolved in pyridine (4 mL) and Et₃N (0.05 mL, 0.7 mmol, 1.5 eq) and activated 3 A molecular sieves were added. The solution was stirred at room temperature for 15 min before DMAP (16 mg, 0.36 mmol, 1.5 eq) and DMT-Cl (204 mg, 0.6 mmol, 2.5 eq) were added. The reaction was left to stir at room temperature for 16 h before the molecular sieves were removed by filtration and the organic solvents removed under vacuum. The resulting residue was purified by column chromatography (0-30% MeOH in EtOAc with a constant additive of 2% pyridine) to yield 9c (114 mg). Rather than risk degradation, the final product was not evaporated to complete dryness and was stored and used with 1 eq of pyridine present (as determined by NMR), making the final yield 60%.

TLC (CH₂Cl₂:MeOH, 3:2 v/v, +2% Et₃N) R_{f}: 0.37;

¹H NMR (500 MHz, DMSO-d₆): δ 8.65-8.45 (m, 2H, pyridine), 8.22-8.12 (m, 2H), 7.89 (s, 1H), 7.78 (tt, J=7.6, 1.9, 1H, pyridine), 7.60 (t, J=7.4, 1H), 7.50 (t, J=7.7, 2H), 7.48-7.42 (m, 2H), 7.44-7.30 (m, 8H, pyridine), 7.29-7.24 (m, 1H), 6.93 (dd, J=8.9, 4.0, 4H), 5.58 (s, 1H), 4.54 (s, 1H), 3.75 (s, 6H), 3.69 (d, J=8.7, 1H), 3.66 (d, J=8.7, 1H), 3.55 (d, J=11.4, 1H), 3.36 (d, J=11.4, 1H), 3.34 (br s, 1H), 2.47 (m, 1H under solvent peak), 2.25 (dd, J=17.0, 8.9, 1H), 2.05 (dd, J=17.0, 4.4, 1H), 1.87-1.75 (m, 3H);

¹³C NMR (126 MHz, d₆-DMSO): δ 177.6, 172.5, 159.2, 149.6 (pyridine), 147.1, 144.6, 137.1, 136.1 (pyridine), 135.2, 135.1, 132.6, 129.8, 129.3, 129.2, 128.4, 128.0, 127.6, 126.9, 123.9 (pyridine), 113.4, 113.3, 109.2, 89.6, 87.3, 85.9, 79.5, 71.3, 58.5, 55.1, 39.4, 28.4, 13.6; HRMS (m/z): [M+H]⁺ calcd. for C₄₁H₄₀N₃O₉⁺, 718.2759, found, 718.2756.

### 5′-O-DMT N⁶-benzoyladenine LNA acid 9d

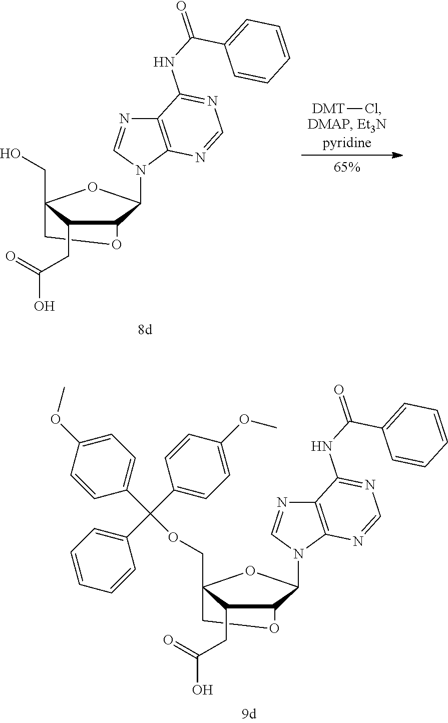

Compound 8d (62.5 mg, 0.15 mmol) was dissolved in pyridine (1.5 mL) and Et₃N (0.031 mL, 0.22 mmol, 1.5 eq) and activated 3 A molecular sieves were added. The solution was stirred at room temperature for 15 min before DMAP (18 mg, 2 mmol, 1.3 eq) and DMT-Cl (100 mg, 0.29 mmol, 2 eq) were added. The reaction was stirred at room temperature for 2 h, and a second portion of DMT-Cl (100 mg, 0.29 mmol, 2.0 eq) was added. After 16 h the molecular sieves were removed by filtration and the organic solvents removed under vacuum. The resulting residue was purified by column chromatography (5-15% MeOH in EtOAc with a constant additive of 2% pyridine) to yield 9d (76 mg). Rather than risk degradation, the final product was not evaporated to complete dryness and was stored and used with 0.72 eq of pyridine present (as determined by NMR), making the final yield 65%.

TLC (EtOAc:MeOH, 3:7 v/v, +2% Et₃N) R_{f}: 0.50;

¹H NMR (500 MHz, DMSO-d₆): δ 12.63 (s, 1H), 11.27 (s, 1H), 8.78 (s, 1H), 8.60-8.55 (m, 1H, pyridine), 8.48 (s, 1H), 8.08-8.02 (m, 2H), 7.78 (tt, J=7.6, 1.9, 0.5H, pyridine), 7.68-7.61 (m, 1H), 7.55 (t, J=7.7, 2H), 7.43-7.22 (m, 10H, includes pyridine), 6.90-6.85 (m, 4H), 6.14 (s, 1H), 4.81 (s, 1H), 3.84 (d, J=8.5, 1H), 3.77 (d, J=8.5, 1H), 3.73 (s, 6H), 3.47 (d, J=11.2, 1H), 3.44 (d, J=11.2, 1H), 2.73 (dd, J=9.7, 4.2, 1H), 2.24-2.16 (m, 1H), 2.08 (dd, J=16.8, 4.2, 1H);

¹³C NMR (126 MHz, d₆-DMSO): δ 173.2, 165.7, 158.2, 151.8, 151.4, 150.4, 149.6 (pyridine), 144.6, 140.8, 133.3, 136.1 (pyridine), 135.2, 135.2, 133.4, 132.5, 129.8, 129.7, 128.5, 128.5, 127.9, 127.6, 126.9, 126.8, 125.6, 123.9 (pyridine), 113.3, 88.8, 85.7, 85.6, 80.0, 71.8, 59.5, 55.0, 41.6, 29.2;

HRMS (m/z): [M+H]⁺ calcd. for C₄₁H₃₈O₈N₅⁺, 728.2711; found, 728.2715.

### 5′-O-DMT N²-isobutyrylguanine LNA acid 9e

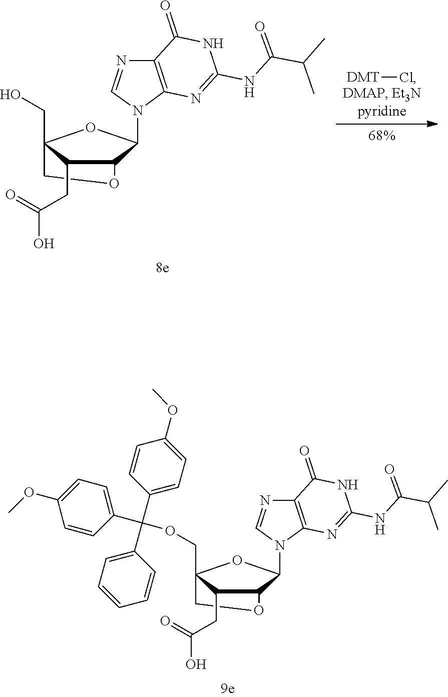

Compound 8e (41 mg, 0.10 mmol) was dissolved in pyridine (1.7 mL) and Et₃N (0.023 mL, 0.17 mmol, 1.7 eq) and activated 3 A molecular sieves were added. The solution was stirred at room temperature for 15 min before DMAP (6.7 mg, 0.05 mmol, 0.5 eq) and DMT-Cl (85 mg, 0.25 mmol, 2.5 eq) were added. The reaction was left to stir at room temperature for 16 h. The molecular sieves were removed by filtration and the organic solvents removed under vacuum. The resulting residue was purified by column chromatography (5-15% MeOH in EtOAc with a constant additive of 2% pyridine) to yield 9e (52 mg). Rather than risk degradation, the final product was not evaporated to complete dryness and was stored and used with 0.65 eq of pyridine present (as determined by NMR), making the final yield 68%.

TLC (EtOAc:MeOH, 3:7 v/v, +2% Et₃N) R_{f}: 0.53;

¹H NMR (500 MHz, DMSO-d₆): δ 8.97-8.40 (m, 1.2H, pyridine), 8.11 (s, 1H), 7.91-7.56 (m, 0.6H, pyridine) 7.39-7.34 (m, 3.4H, with 1.4H from pyridine), 7.30 (dd, J=8.6, 6.9, 2H), 7.29-7.17 (m, 5H), 6.93-6.84 (m, 4H), 5.85 (s, 1H), 4.71 (s, 1H), 3.76 (d, J=8.4, 1H), 3.73 (s, 6H), 3.71 (d, J=8.4, 1H), 3.41 (d, J=11.1, 1H), 3.31 (d, J=11.1, 1H), 3.16 (s, 1H, MeOH), 2.79 (p, J=6.9, 1H), 2.67 (dd, J=10.2, 3.9, 1H), 2.10-1.94 (m, 2H), 1.11 (dd, J=6.9, 1.9, 6H);

¹³C NMR (126 MHz, d₆-DMSO): δ 180.2, 175.1 (broad), 158.2, 154.9, 148.4, 148.0, 144.7, 136.0, 135.2, 135.1, 129.7, 127.9, 127.6, 126.8, 120.4, 113.3, 113.3, 88.7, 85.6, 85.1, 80.1, 71.9, 59.5, 55.1, 42.5, 34.7, 30.4, 18.9, 18.9;

HRMS (m/z): [M+H]⁺ calcd. for C₄₀H₄O₉N₅⁺, 710.2821; found 710.2817.

### N₃ Thymine LNA S14¹

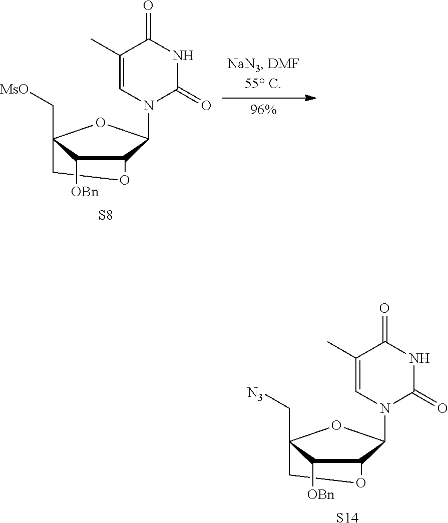 [SMILES: Cc1cn([C@@H]2O[C@@]3(COS(C)(=O)=O)COC2[C@H]3OCc2ccccc2)c(=O)[nH]c1=O ; "NaN3, DMF55° C." ; "S8" ; Cc1cn([C@@H]2O[C@@]3(CN=[N+]=[N-])COC2[C@H]3OCc2ccccc2)c(=O)[nH]c1=O ; "S14"]

Compound S14 was prepared based on a similar procedure outlined by Thorpe et al.¹ Compound S8² (3.5 g, 8.0 mmol) and NaN₃ (1.04 g, 16 mmol, 2 eq) were dissolved in DMF (40 mL) and the reaction was stirred at 50° C. for 5 h. Sodium azide is potentially explosive if handled in correctly. The solvent was removed under vacuum and the resulting residue was partitioned between EtOAc (40 mL) and water (40 mL). The organic layer was washed with water (2×40 mL), dried over Na₂SO₄, and evaporated to dryness to yield S14 (2.96 g, 7.7 mmol) as a white solid in 96% yield which was used without purification. If required the compound can be purified by column chromatography (50-100% EtOAc in 40-60 PE).

Data consistent with literature¹.

### Amino thymine LNA S11¹

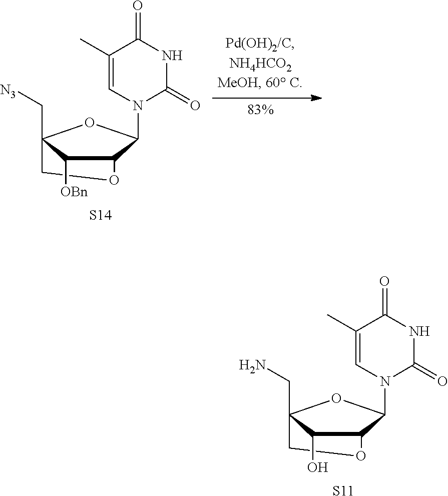 [SMILES: Cc1cn([C@@H]2O[C@@]3(CN=[N+]=[N-])COC2[C@H]3OCc2ccccc2)c(=O)[nH]c1=O ; "Pd(OH)2/C,NH4HCO2MeOH, 60° C." ; "S14" ; Cc1cn([C@@H]2O[C@@]3(CN)COC2[C@H]3O)c(=O)[nH]c1=O ; "S11"]

Compound S11 (2.0 g, 5.2 mmol) and ammonium formate (4.0 g, 63 mmol, 12 eq) were dissolved in MeOH (100 mL) and 20 wt % palladium hydroxide on carbon (0.36 g, 0.52 mmol, 10 mol %) was added. The flask was flushed with argon and the reaction was stirred at 60° C. for 4 h. A large volume of gas is generated within the first hour of the reaction presenting a risk of over-pressurisation. The reaction was filtered through celite to remove the catalyst and the solvent was removed under vacuum. The resulting solid was purified by column chromatography (0-30% MeOH in EtOAc) to give S11 (1.17 g, 4.3 mmol) as a white solid in 83% yield.

Data consistent with literature¹.

### 5′-N-MMT thymine LNA S12³

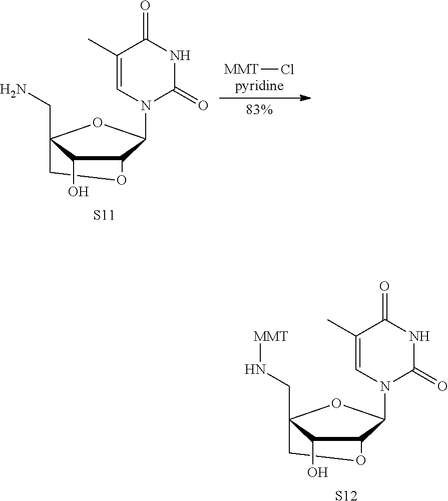

Amine S11 (1.17 g, 4.3 mmol) was dissolved in anhydrous pyridine (50 mL) and 4-methoxytriphenylmethyl chloride (1.6 g, 5.2 mmol, 1.2 eq) was added in small portions. The reaction was stirred at room temperature for 2 h before the solvents were removed under vacuum. The resulting residue was purified by column chromatography (0-30% EtOAc in 40-60 PE with a constant additive of 0.1% pyridine) to give S12 (1.93 g, 3.6 mmol) as a pale-yellow foam in 83% yield.

TLC (EtOAc:hexane, 3:2 v/v, +0.5% pyridine) R_{f}: 0.5;

¹H NMR (400 MHz, CDCl₃): δ 9.32 (s, 1H), 7.65 (s, 1H), 7.48-7.45 (m, 4H), 7.38-7.37 (m, 2H), 7.29-7.24 (m, 4H), 7.17 (t, J=7.3, 2H), 6.81 (d, J=9.0, 2H), 5.61 (s, 1H), 4.46 (s, 1H), 4.26 (s, 1H), 4.00 (br s, 1H), 3.92 (d, J=8.3, 1H), 3.78 (d, J=8.2, 1H), 3.74 (s, 3H), 2.65-2.49 (m, 2H), 2.09 (t, J=8.5, 1H), 1.92 (d, J=1.2, 3H).

¹³C NMR (101 MHz, CDCl₃): δ 164.1, 158.3, 150.0, 145.9, 145.8, 137.4, 134.7, 129.9, 128.5, 128.2, 126.8, 113.5, 110.5, 88.8, 87.2, 79.8, 72.7, 70.7, 70.4, 55.4, 40.2, 12.8;

HRMS (m/z): [M+Na]⁺ calcd. for C₃₁H₃₁O₆N₃Na⁺, 564.2105, found, 564.2103;

No spectroscopic data reported previously³.

### 5′-N-MMT thymine LNA phosphoramidite 103

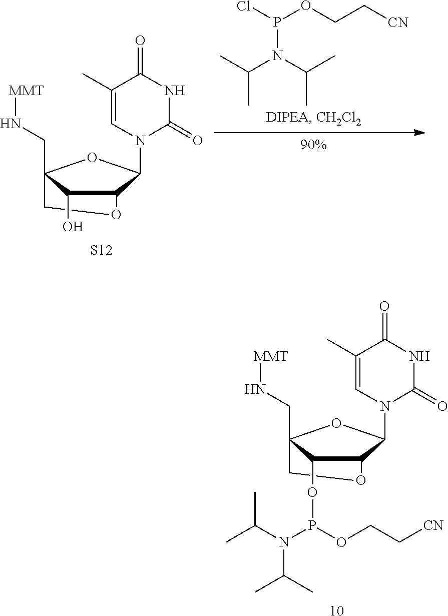

Nucleoside 10 (1.1 g, 2.0 mmol) was dissolved in anhydrous degassed CH₂Cl₂ (10 mL). Degassed N,N-diisopropylethylamine (DIPEA) (883 μL, 5.1 mmol, 2.5 eq) and 2-cyanoethyl N,N-diisopropylchlorophosphoramidite (677 μL, 3.0 mmol, 1.5 eq) were added and the reaction was stirred under an argon atmosphere at room temperature for 2 h. The reaction mixture was diluted with CH₂Cl₂ (40 mL) and washed with a saturated aqueous solution of KCl (30 mL). The organic phase was dried over Na₂SO₄ and the solvents were removed under vacuum. The resulting pale-yellow oil was purified by column chromatography (40% EtOAc in hexane with a constant additive of 0.5% pyridine) to give the phosphoramidite 10 (1.3 g, 1.8 mmol) as a white foam in 90% yield.

TLC (EtOAc:hexane, 2:3 v/v, +0.5% pyridine) R_{f}: 0.4;

³¹P NMR (162 MHz, CDCl₃): δ 148.7, 148.3

HRMS (m/z): [M−H]⁻ calcd. for C₄₀H₄₇O₇N₅P⁻, 740.3219; found 740.3219.

### Oligonucleotide Synthesis

### DNA Synthesis and Cleavage

DNA synthesis was performed on an Applied Biosystems 394 automated DNA/RNA synthesiser using a standard phosphoramidite cycle of detritylation, coupling, capping (unless stated elsewhere), and oxidation on a 1.0 μmole scale. Trichloroacetic acid (TCA) (3% in CH₂Cl₂) was used for detritylation, 5-benzylthio-1H-tetrazole (BTT) (0.25 M in MeCN) was used as an activator, and oxidation was achieved using iodine (0.02 M in THF, pyridine and water). Pre-packed nucleoside SynBase™ CPG 1000/110 (Link Technologies) were used and β-cyanoethyl phosphoramidite monomers (dA(Bz), dG(iBu), dC(Bz) and dT, Sigma-Aldrich) were dissolved in anhydrous MeCN (0.1 M) immediately prior to use with coupling time of 50 s. LNA β-cyanoethyl phosphoramidite monomers (QIAGEN) were dissolved to a concentration of 0.1 M in either MeCN (LNA-T) or 25% THF/MeCN (LNA-mC(Bz)) immediately prior to use with a coupling time of 6 min. Stepwise coupling efficiencies were determined by automated trityl cation conductivity monitoring and were >98% in all cases. Cleavage and deprotection were achieved by exposure to concentrated aqueous ammonia solution for 60 min at room temperature followed by heating in a sealed tube for 5 h at 55° C.

### RNA Synthesis and Cleavage

RNA synthesis was performed on an Applied Biosystems 394 automated DNA/RNA synthesiser using a standard phosphoramidite cycle of detritylation, coupling, capping, and oxidation on a 1.0 μmole scale. Coupling, capping and oxidation reagents were identical to those used for DNA synthesis except a solution of ethylthiotetrazole (ETT) (0.25 M in MeCN, Link Technologies) was used instead of BTT as the activator. Standard CPG resin (Link Technologies) was used and 2′-thiomorpholine-4-carbothioate (TC) protected monomers (A(Bz), C(Ac), G(iBu) and U, Sigma-Aldrich) were dissolved in anhydrous toluene/MeCN (1:1 v/v, 0.1 M) immediately prior to use. The coupling time for all monomers was 3 min. Stepwise coupling efficiencies were determined by automated trityl cation conductivity monitoring and in all cases were >97%. To deprotect and cleave the RNA, the solid support was exposed to dry ethylenediamine:toluene (1:1 v/v) for 6 h at room temperature, washed with toluene (3×1 mL), then MeCN (3×1 mL) and dried using argon. The cleaved RNA was eluted from the solid support with water.

### 2′OMe Phosphodiester Oligonucleotide Synthesis and Cleavage

2′OMe oligonucleotides were synthesised on an Applied Biosystems 394 automated DNA/RNA synthesiser using a standard phosphoramidite cycle of detritylation, coupling (unless otherwise stated), capping, and oxidation on a 1.0 μmole scale. Detritylation, coupling, capping, oxidation and activation reagents are identical to those used for DNA synthesis. Pre-packed nucleoside SynBase™ CPG 1000/110 (Link Technologies) were used, and β-cyanoethyl phosphoramidite monomers (DMT-2′O-Methyl-rA(Bz), DMT-2′O-Methyl-rG(iBu), DMT-2′O-Methyl-rC(Ac) and DMT-2′O-Methyl-rU, Sigma-Aldrich) were dissolved in anhydrous MeCN (10% CH₂Cl₂ was added when 2′OMe U phosphoramidite was used) to a concentration of 0.1 M immediately prior to use with a coupling time of 6 min. LNA β-cyanoethyl phosphoramidite monomers (QIAGEN) were dissolved to a concentration of 0.1 M in either MeCN (LNA-T) or 25% THF/MeCN (LNA-mC(Bz)) immediately prior to use with a coupling time of 6 min. Stepwise coupling efficiencies were determined by automated trityl cation conductivity monitoring and were >98% in all cases. Cleavage and deprotection were achieved by exposure to concentrated aqueous ammonia solution for 60 min at room temperature followed by heating in a sealed tube for 5 h at 55° C.

### Phosphodiester Oligonucleotide Purification

Oligonucleotides were purified using a Gilson reverse-phase high performance liquid chromatography (RP-HPLC) system with ACE® C8 column (particle size: 10 μm, pore size: 100 Å, column dimensions: 10 mm×250 mm) with a gradient of buffer A (0.1 M TEAB, pH 7.5) to buffer B (0.1 M TEAB, pH 7.5 containing 50% v/v MeCN) and flow rate of 4 mL/min. The gradient of MeCN in triethylammonium bicarbonate (TEAB) was increased from 0% to 50% buffer B over 30 min. Elution was monitored by UV absorbance at 298 nm. After HPLC purification, oligonucleotides were freeze dried then dissolved in water without the need for desalting.

### Phosphorothioate Oligonucleotide Synthesis, Cleavage and Purification

Oligonucleotides with a phosphorothioate rather than a phosphodiester backbone were synthesised as described above, except for a solution of 3-ethoxy-1,2,4-dithiazoline-5-one (EDITH, Link Technologies) in MeCN (0.05 M) was used as a sulfurising reagent in place of the oxidising solution. The sulfurisation time was extended to 3 min followed by sending fresh EDITH to the synthesis column and leaving it for another 3 min. Phosphorothioate modified oligonucleotides were isolated with the final 5′-DMT protecting group still in place (DMT-On). Following solid phase synthesis, the cyanoethyl groups were removed by a 15 min treatment with 20% diethylamine in MeCN. The resin was then washed with MeCN (5×1 mL) and dried by passing a stream of argon through the synthesis column. The oligonucleotides were cleaved from the solid support and deprotected by heating in a sealed glass vial at 55° C. for 5 h. The ammonia was removed under reduced pressure prior to oligonucleotide purification. The DMT-On oligonucleotides were purified by RP-HPLC and lyophilised. They were then dissolved in 0.5 mL of 80% acetic acid and incubated for 1 h at room temperature to remove the DMT group. The solution was neutralised with 0.5 mL of triethylammonium acetate buffer (2 M, pH 7) and the detritylated oligonucleotides were desalted using a NAP-10 column (Cytiva) then freeze dried.

### Oligonucleotide Analysis

All oligonucleotides were characterised by negative-mode ultra-performance liquid chromatography (UPLC) mass spectrometry using a Waters Xevo G2-XS QT of mass spectrometer with an Acquity UPLC system, equipped with an Acquity UPLC oligonucleotide BEH C18 column (particle size: 1.7 μm; pore size: 130 A; column dimensions: 2.1 mm×50 mm). Data were analysed using Waters MassLynx software or Waters UNIFI Scientific Information System software.

### LNA-Amide Modified Oligonucleotide Synthesis

### Oligonucleotide Segment Synthesis

Oligonucleotide segments were synthesised as described, except that the capping step was omitted.

### Amino Monomer Addition

The MMT-protected 5′-amino phosphoramidite monomer (either LNA 10³ or commercially available deoxythymidyl 11) was dissolved in anhydrous MeCN (0.1 M) immediately prior to use. The same conditions as above were used, but the coupling time was extended to 10 min. No capping step was used. The 5′-MMT protecting group was cleaved on the Applied Biosystems 394 automated synthesiser using TCA (3% in CH₂Cl₂) with an extended cleavage time of 2 min. The solid support was then washed with MeCN on the synthesiser for 3 min. To improve the coupling efficiency in the next step the solid support was washed with N-methylmorpholine in DMF (0.5% v/v, 1 mL) followed by DMF (3×1 mL).

### Amide Bond Formation on Resin (Peptide Coupling)

All amide couplings were performed manually in the synthesis column. A solution with 10 equivalents of acid monomer, 10 equivalents of PyBOP and 30 equivalents of N-methylmorpholine was first prepared in 400 μL of DMF. This was then taken up into a 1 mL syringe and loaded into the column before a second 1 mL syringe was attached to the other end of the synthesis column. The mixture was agitated every 10 min for 1 h. The columns were then washed with DMF (3×1 mL) followed by MeCN (5×1 mL) and dried by passing argon through the column. The column was then returned to the synthesiser to continue oligonucleotide synthesis.

Cleavage of Oligonucleotides from Resin, Deprotection and Purification

LNA-amide containing oligonucleotides were isolated with the final 5′-DMT protecting group still in place (DMT-On). Following solid phase synthesis, the cyanoethyl groups were removed by a 15 min treatment with 20% diethylamine in MeCN. The resin was then washed with MeCN (5×1 mL) and dried by passing a stream of argon through the synthesis column. The oligonucleotides were cleaved from the solid support and deprotected by heating in concentrated aqueous ammonia solution in a sealed glass vial at 55° C. for 5 h. The ammonia was removed under reduced pressure prior to oligonucleotide purification. The DMT-On oligonucleotides were purified by RP-HPLC. The elution of oligonucleotides was monitored by UV absorbance at 298 nm. The oligonucleotides were lyophilised and then dissolved in 0.5 mL of 80% acetic acid, and incubated for 1 h at room temperature to remove the DMT group. The solution was neutralised with 0.5 mL of triethylammonium acetate buffer (2 M, pH 7) and the detritylated oligonucleotides were desalted using a NAP-10 column (Cytiva), then freeze dried.

### Biophysical Studies

### UV Melting Experiments

UV melting experiments were performed using a Cary 4000 scan UV-vis spectrophotometer. 3 nmol of each oligonucleotide was dissolved in 1 mL of 10 mM phosphate buffer containing 200 mM NaCl at pH 7.0. The samples were first denatured by heating to 85° C. (10° C./min) and then annealed by slowly cooling to 20° C. (1° C./min). Six successive cycles of heating and cooling were performed at a gradient of 1° C./min whilst recording the change in UV absorbance at 260 nm. The built-in software was then used to calculate the melting temperature from the first derivative of the melting curve.

### Oligonucleotide X-Ray Crystallography

### Crystallisation

DNA and RNA oligonucleotides were purified by HPLC, desalted by gel filtration (NAP-10) and then freeze dried. Oligonucleotide stock solutions (2 mM) were prepared in aqueous KCl (10 mM). DNA samples were combined with an equimolar ratio of complementary RNA to form their respective modified DNA:RNA hybrids to form 1 mM duplex (60 μL). Single crystals of the DNA:RNA duplexes were obtained by the sitting drop vapour diffusion method. The Natrix HT sparse matrix screen (Hampton Research, HR2-131) was used to identify crystallisation hits for each modified duplex sample using high throughput (HT) methods. All HT screens were performed in CrystalMation Intelli-Plate 96-3 low-profile plates (Hampton Research, HR3-119). Reservoirs and drops were dispensed using an Art Robbins Phoenix automatic liquid handler. Reservoirs contained 80 μL of Natrix HT solution and crystallisation drops (200-300 nL total volume) were placed in each of the three subwells; subwell 1, 200 nL oligo:100 nL well solution; subwell 2, 100 nL oligo: 100 nL well solution; subwell 3, 100 nL oligo: 200 nL well solution (stock duplex concentration was 1 mM). Plates were sealed using optically clear Xtra-Clear Advanced Polyolefin StarSeal (StarLab) and incubated at 19° C., crystals usually formed within one week (range 2-90 days, crystal size <10-200 m). The unmodified DNA:RNA duplex was crystallised using adapted conditions from Kopka et al.⁴ Optimisation of these conditions were done in 24 well Cryschem sitting drop plates (Hampton Research, USA) using 4 μL sitting drops consisting of 0.5 mM duplex, 12 mM Mg(OAc)₂, 0.6 mM spermidine·HCl, 0.075% (w/v) β-octylglucoside, 12 mM sodium cacodylate and 12% 2-methyl-2,4-pentanediol (MPD). This was equilibrated against a reservoir of H₂O:MPD (1:1 v/v, 400 μL). To screen conditions, components of the drop were varied (6-16 mM Mg(OAc)₂, 0.2-1.2 mM spermidine·HCl, 0.075% (w/v) β-octylglucoside, 12 mM sodium cacodylate and 6-16% MPD. All other structures were obtained using hits from the NatrixHT screen (Hampton Research, USA). All samples were crystallised at 19° C. using the conditions outlined in Table S6.

### Data Collection and Processing

Sample wells were opened and cryo protectant 20% glycerol in reservoir solution (2 μL) was added. Crystals were harvested using cryoloops (0.01-0.05 mm) and immediately cryo-cooled by plunging into liquid N₂ (77 K), transferred into a cryo-vial and stored under liquid nitrogen at 77 K until data collection. Data collection was performed at Diamond Light Source (beamlines i03 or i04) or DESY in Hamburg (beamline P13). The high radiation damage resistance of the oligo duplex crystals permitted 100% beam transmission. Oscillation images (3600 images, 0.1° osc) were collected. The detector distance was set to obtain a maximum resolution of 0.5 A greater than the expected diffraction limit to maximise spot separation (see Table S5) and reduce overlapping reflections and obtain maximal completeness. Data were auto processed using either fast dp⁵, xia2_dials⁶ or xia2_3dii⁷. CC_{1/2}>0.3 and completeness >90%, crystal data quality was reviewed using Phenix.Xtriage. ON26^{xDNA}, ON29^{xDNA-Am-DNA}, and ON29^{xLNA-Am-DNA} duplexes all crystallised in the high symmetry space group P6₁ and contained a single DNA:RNA hybrid in the asymmetric unit. In contrast, the ON^{30xLNA-Am-LNA} duplex was in the lower symmetry Space Group P 3₂21 with two DNA:RNA hybrids in each asymmetric unit.

### Structure Solution, Model Building and Refinement

The structures were solved using the Molecular Replacement method and 1PJO PDB ID as the search model^{6, 9} using PHASER 2.8.2¹⁰. Structure solutions resulted in TFZ score>8.0 and LLG>50 and correct solution was confirmed by visual inspection of electron density maps. The DNA:RNA models (some with modified backbone) were built and fit to the electron density using winCOOT¹¹. Model refinement was performed using REFMAC5¹² and PHENIX.REFINE¹³. Geometric restraints for the non-standard phosphoribosyl backbones were generated using JLIGAND⁸ or ACEDRG¹⁴. Model building continued until the observed electron density was satisfied and the R_{free} no longer decreased. Software packages and project management was handled using CCP4¹⁵ and Phenix¹³. Images were made using PYMOL graphic software (The PyMOL Molecular Graphics System, Version 2.3.2 Schrödinger, LLC).

Where necessary, data were reprocessed to achieve acceptable final statistics (i.e. CC ½>0.3). Reprocessing was performed using iMosflm, XDS or in-house using automated xia2 pipelines⁷. The data were then scaled and merged using Aimless¹⁶.

### Biological Assays

### Evaluation of Stability in Fetal Bovine Serum (FBS)

Five nmol of each oligonucleotide was dissolved in Dulbecco's PBS (50 μL) and FBS (50 μL, Gibco, standard sterile-filtered) was added. The sample was mixed by pipetting and 20 μL of this solution was immediately removed, mixed with formamide (20 μL), snap frozen in liquid N₂, and stored at −80° C. as a control (0 h). The remaining reaction mixtures were incubated at 37° C. and aliquots (20 μL) were taken at different time intervals, mixed with formamide (20 μL), snap frozen in liquid N₂ and stored at −80° C. The samples were then analysed by denaturing 20% polyacrylamide gel.

### Cell Culture

HeLa pLuc/705 cells¹⁷ were cultured in Dulbecco's Modified Eagle Medium with GlutaMAX-I (Gibco) supplemented with 10% (v/v) FBS (Gibco) and 1×Antibiotic-Antimycotic (Gibco) at 37° C. in a humidified incubator with 5% CO₂.

Transfection with Lipofectamine 2000

Cells were seeded at a density of 7000 cells/well in 100 μL of culture media in 96 well plates 16 h before transfection to reach 70-80% cell confluency. Immediately prior to transfection, 5 μL of Lipofectamine 2000 (Invitrogen) was diluted in 500 μL OptiMEM (Gibco) and incubated at room temperature for 5 min before mixing with 4 pmol of lyophilised oligonucleotide dissolved in 500 μL of OptiMEM. The resulting mixture was incubated at room temperature allowing complexation to occur. The complexes were then further diluted in OptiMEM to the concentrations required for the experiments. Culture media was removed from the cells and 100 μL of the complexes added per well. The cells were then incubated at 37° C. in a humidified incubator with 5% CO₂. After 4 h the media was replaced with 100 μL of culture media and the cells were returned to the incubator for a further 20 h.

### Gymnosis Experiments

Cells were seeded at a density of 800 cells/well in 100 μL in culture media in 96 well plates 16 h before the oligonucleotides were added. Lyophilised oligonucleotides were dissolved in OptiMEM immediately before addition to the cells. The media in each well was removed and replaced with 100 μL of the oligonucleotide containing OptiMEM. The cells were then incubated for 96 h at 37° C. in a humidified incubator with 5% CO₂.

### Luciferase Assay

The culture media was removed from the well and the cells were washed with 200 μL of PBS. 100 μL of GloLysis™ buffer (Promega) was added to each well. The plate was incubated at room temperature on the orbital shaker for 10 min to lyse the cells. 50 μL of the cell lysate was added to 50 μL of Bright-Glo™ luciferase reagent (Promega) in a white 96 well plate and the luminescence was measured using a Clariostar plate reader. 25 μL of the cell lysate was then used for protein quantification using a Pierce BCA protein assay kit in accordance with the manufacturer's guidelines, using GloLysis buffer as a blank standard. The luminescence values were divided by the total protein quantities and normalised to the values for untreated cells.

### WST-1 Cell Viability Assay

The cell viability was evaluated using the WST-1 cell proliferation reagent (Roche) in accordance with the manufacturer's guidelines. Briefly, cells were seeded, transfected using Lipofectamine 2000, and the media was changed to culture media after 4 h, as described above. The cells were then incubated for 20 h at 37° C. in a humidified incubator with 5% CO₂ before WST-1 reagent (10 μL) was added to each well. The cells were returned to the incubator for 4 h. The cells were shaken at 500 rpm for 1 min before 10 μL of media was removed from each well and added to a clear 96 well plate containing 90 μL of water in each well and the absorbance at 440 nm was measured using a ClarioStar plate reader. This dilution step was necessary as the absorbance went above the accurate range of the instrument. Cells that were treated with OptiMEM instead of the oligonucleotide complexes were used as a 100% viability reference.

### Supplementary Tables

TABLE S1
List of oligonucleotides (ONs) used in this study with calculated and found mass
spectrometry (MS) data. UPLC-MS traces for all ONs are given.

| | | | | |
|---|---|---|---|---|
| ON | Sequence (5′ → 3′) | Backbone | Expected | Found |

Single amide addition UV melting studies

| | | | | |
|---|---|---|---|---|
| ON1^{DNA-Am-DNA} | CGACGCT*TGCAGC | DNA | 3896.7 | 3897.0 |
| ON2^{LNA-Am-DNA} | CGACGCT*TGCAGC | DNA | 3924.8 | 3925.0 |
| ON3^{DNA-Am-LNA} | CGACGCT*TGCAGC | DNA | 3924.8 | 3925.0 |
| ON4^{LNA-Am-LNA} | CGACGCT*TGCAGC | DNA | 3952.9 | 3953.0 |
| ON5^{LNA-LNA} | CGACGCTTGCAGC | DNA | 3995.5 | 3992.0 |
| ON6^{DNA-DNA} | CGACGCTTGCAGC | DNA | 3935.5 | 3936.0 |

Single amide addition UV melting targets

| | | | | |
|---|---|---|---|---|
| ON7 | GCTGCAAGCGTCG | DNA | 3975.5 | 3976.0 |
| ON8 | GCUGCAAGCGUCG | RNA | 4155.2 | 4156.5 |
| ON9 | GCUGCA [symbol] GCGUCG | RNA | 4131.2 | 4132.0 |
| ON10 | GCUGC [symbol] AGCGUCG | RNA | 4131.2 | 4132.0 |
| ON11 | GCUGCA [symbol] GCGUCG | RNA | 4171.2 | 4171.5 |
| ON12 | GCUGC [symbol] AGCGUCG | RNA | 4171.2 | 4172.4 |

Multiple amide addition in different backbones

| | | | | |
|---|---|---|---|---|
| ON13^{DNA/1LAL/16PO} | CCTCTTACC*TCAGTTACA | DNA, PO | 5386.8 | 5387.5 |
| ON14^{DNA/4LAL/13PO} | CC*TCT*TACC*TCAGT*TACA | DNA, PO | 5438.7 | 5439.0 |
| ON15^{DNA/17PO} | CCTCTTACCTCAGTTACA | DNA, PO | 5369.6 | 5370.0 |
| ON16^{2′OMe/4LAL/13PO} | C^{Me}C*TCT*TAC^{Me}C*TCAGT*T | 2′OMe, PO | 5766.9 | 5767.0 |
| | ACA | | | |
| ON17^{2′OMe/17PO} | CCUCUUACCUCAGUUACA | 2′OMe, PO | 5824.7 | 5825.0 |
| ON18^{2′OMe/4LAL/13PS} | C^{Me}C*TCT*TAC^{Me}C*TCAGT*T | 2′OMe, PS | 5976.2 | 5975.5 |
| | ACA | | | |
| ON19^{2′OMe/8LNA/17PS} | C^{Me}CTCTTAC^{Me}CTCAGTTAC | 2′OMe, PS | 6193 | 6195.7 |
| | A | | | |
| ON20^{2′OMe/17PS} | CCUCUUACCUCAGUUACA | 2′OMe, PS | 6096.3 | 6097.6 |

Multiple amide addition in different backbones UV melting targets

| | | | | |
|---|---|---|---|---|
| ON21 | TGTAACTGAGGTAAGAGG | DNA | 5627.6 | 5628.5 |
| ON22 | UGUAACUGAGGUAAGAGG | RNA | 5858.9 | 5859.0 |
| ON23 | AGGTAAGAGG | DNA | 3141.0 | 3141.5 |
| ON24 | AGGUAAGAGG | RNA | 3286.8 | 3288.0 |

Additional digestion oligonucleotide

| | | | | |
|---|---|---|---|---|
| ON25^{DNA/8LNA/17PO} | C^{Me}CTCTTAC^{Me}CTCAGTTAC | DNA | 5621.6 | 5622.8 |
| | A | | | |

XRD oligonucleotides

| | | | | |
|---|---|---|---|---|
| ON26^{xDNA} | CTTTTCTTTG | DNA | 2974.9 | 2976.0 |
| ON27^{xRNA} | CAAAGAAAAG | RNA | 3238.0 | 3240.0 |
| ON28^{xDNA-Am-DNA} | CTT*TTCTTTG | DNA | 2936.1 | 2937.0 |
| ON29^{xLNA-Am-DNA} | CTT*TTCTTTG | DNA | 2964.2 | 2964.5 |
| ON30^{xLNA-Am-LNA} | CTT*TTCTTTG | DNA | 2992.3 | 2992.5 |

Scrambled control splice-switching oligonucleotide

| | | | | |
|---|---|---|---|---|
| ON31^{2′OMe/17PS} | CCUCAUUCACUCGAUUCA | 2′OMe, PS | 6096.3 | 6099.7 |
| scrambled | | | | |

Supplementary Oligonucleotides

| | | | | |
|---|---|---|---|---|
| ONS1 | CGACGCC*TGCAGC | DNA | 3937.8 | 3938.5 |
| ONS2 | CGACGC^{Me}C*TGCAGC | DNA | 3951.9 | 3953.0 |
| ONS3 | CGACGCA*TGCAGC | DNA | 3961.9 | 3963.0 |
| ONS4 | CGACGCG*TGCAGC | DNA | 3977.9 | 3978.5 |

Underlined bases indicates a locked sugar; * is an amide bond in place of a phosphodiester, underlined italic and highlighted bases indicates the position of the mismatch. Backbone denotes to the chemistry of inter-sugar linkages and the sugars not flanking an amide bond.

Underlined bases indicates a locked sugar; * is an amide bond in place of a phosphodiester, underlined italic and highlighted bases indicates the position of the mismatch. Backbone denotes to the chemistry of inter-sugar linkages and the sugars not flanking an amide bond.

TABLE S2
Comparison of the melting temperatures of duplexes containing a single amide
substitution of the phosphodiester backbone flanked by LNA on the 5′, 3′ or both sides within a
DNA backbone hybridised to DNA or RNA.

| | | DNA | RNA | | | | |
|---|---|---|---|---|---|---|---|
| | | match | match | RNA 5′ C | RNA 3′ C | RNA 5′ G | RNA 3′ G |
| | | Tₘ | Tₘ | mismatch | mismatch | mismatch | mismatch |
| | | (ΔTₘ) | (ΔTₘ) | Tₘ (ΔTₘ) | Tₘ (ΔTₘ) | Tₘ (ΔTₘ) | Tₘ (ΔTₘ) |
| ON | Sequence | ON7 | ON8 | ON9 | ON10 | ON11 | ON12 |
| ON1 | CGACGCT*TGCAGC | 61.2 | 61.0 | 47.0 | 50.2 | 57.3 | 56.6 |
| ^{DNA-} | | (−2.3) | (−0.4) | (−14.1) | (−10.8) | (−3.7) | (−3.4) |
| ^{Am-} | | | | | | | |
| ^{DNA} | | | | | | | |
| ON2 | CGACGCT*TGCAGC | 63.1 | 64.4 | 50.2 | 51.0 | 58.5 | 60.3 |
| ^{LNA-} | | (−0.3) | (+3.0) | (−14.2) | (−13.4) | (−5.9) | (−4.1) |
| ^{Am-} | | | | | | | |
| ^{DNA} | | | | | | | |
| ON3 | CGACGCT*TGCAGC | 60.9 | 63.9 | 50.1 | 52.5 | 58.8 | 56.9 |
| ^{DNA-} | | (−2.5) | (+2.5) | (−13.8) | (−11.4) | (−5.1) | (−7.0) |
| ^{Am-} | | | | | | | |
| ^{LNA} | | | | | | | |
| ON4 | CGACGCT*TGCAGC | 63.4 | 66.5 | 53.3 | 55.1 | 59.8 | 60.5 |
| ^{LNA-} | | (−0.1) | (+5.1) | (−13.2) | (−11.4) | (−6.7) | (−6.0) |
| ^{Am-} | | | | | | | |
| ^{LNA} | | | | | | | |
| ON5 | CGACGCTTGCAGC | 69.6 | 74.1 | 59.7 | 59.9 | 68.6 | 67.3 |
| ^{LNA-} | | (+6.1) | (+12.7) | (−14.4) | (−14.2) | (−5.5) | (−2.3) |
| ^{LNA} | | | | | | | |
| ON6 | CGACGCTTGCAGC | 63.5 | 61.4 | 46.2 | 51.2 | 57.4 | 57.4 |
| ^{DNAcontrol} | | | | (−15.2) | (−10.2) | (−4.0) | (−4.0) |

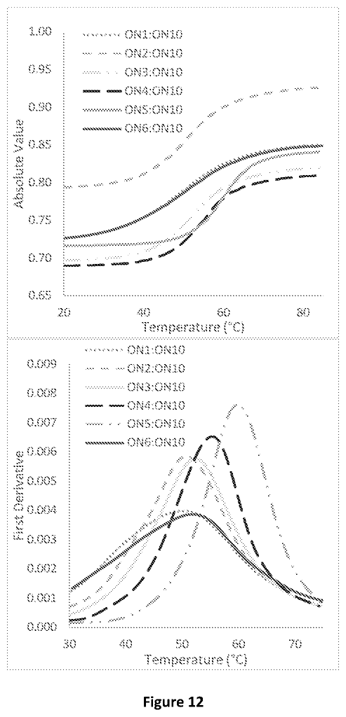

Tₘ values were measured using 3.0 μM concentrations of each oligonucleotide strand in 10 mM phosphate buffer (pH 7.0) containing 200 mM NaCl. Underlined, e.g. T indicates a locked sugar and * is an amide bond in place of a phosphodiester. Tₘ values were calculated as the maximum of the first-derivative of the melting curve (A₂₆₀ vs T) and reported as the average of at least two independent experiments. ΔTₘ for matched sequences=modified−ON6^{DNAcontrol}; ΔTₘ for mismatched=Match−RNA mismatch. Target ON sequences, where X denotes the mismatch. ON7=GCTGCAAGCGTCG; ON8=GCUGCAAGCGUCG; ON9=GCUGCACGCGUCG; ON10=GCUGCCAGCGUCG; RNA ON11=GCUGCAGGCGUCG. ON12=GCUGCGAGCGUCG. Representative melting curves are given (FIG. 9-14).

TABLE S3
Comparison of the relative melting temperatures of duplexes containing 0, 1
or 4 amide linkages flanked by LNA on both sides hybridised to DNA or RNA.

| | | | DNA | RNA |
|---|---|---|---|---|
| ON | Sequence (5′ → 3′) | backbone | Tₘ (ΔTₘ) | Tₘ (ΔTₘ) |
| a | | | | |
| ON13^{DNA/1LAL/16PO} | CCTCTTACC*TCAGTTACA | DNA, PO | 60.9 | 65.8 |
| | | | (+1.8) | (+2.2) |
| ON14^{DNA/4LAL/13PO} | CC*TCT*TACC*TCAGT*TACA | DNA, PO | 64.2 | 76.6 |
| | | | (+5.1) | (+13.0) |
| ON15^{DNA/17PO} | CCTCTTACCTCAGTTACA | DNA, PO | 59.1 | 63.6 |
| b | | | | |
| ON14^{DNA/4LAL/13PO} | CC*TCT*TACC*Tcagt*taca | DNA, PO | 46.5 | 64.3 |
| | | | (+7.0) | (+17.5) |
| ON15^{DNA/17PO} | CCTCTTACCTcagttaca | DNA, PO | 39.5 | 46.8 |
| c | | | | |
| ON16^{2′OMe/4LAL/17PO} | CMeC*TCT*TACMeC*Tcagt*taca | 2′OMe, | 53.2 | 72.3 |
| | | PO | (+19.1) | (+9.3) |
| ON17^{2′OMe/17PO} | CCUCUUACCUcaguuaca | 2′OMe, | 34.1 | 63.0 |
| | | PO | | |
| d | | | | |
| ON18^{2′OMe/4LAL/13PS} | CMeC*TCT*TACMeC*Tcagt*taca | 2′OMe, | 48.1 | 67.4 |
| | | PS | (+25.4) | (+10.3) |
| ON19^{2′OMe/8LNA/17PS} | CMeCTCTTACMeCTcagttaca | 2′OMe, | 59.0 | >80 |
| | | PS | (+36.3) | |
| ON20^{2′OMe/17PS} | CCUCUUACCUcaguuaca | 2′OMe, | 22.7 | 57.1 |
| | | PS | | |

Experimental conditions as in Table S2. a=comparison of DNA ONs against full length targets, b=comparison of DNA ONs against 10-mer targets, c=comparison of 2′OMe/PO ONs, d=comparison of 2′OMe/PS ONs. Backbone: PO=phosphodiester, PS=phosphorothioate, DNA=deoxyribose sugars, 2′OMe=2′OMe RNA sugars. X indicate a locked sugar and * indicates an amide bond in place of a phosphodiester, LAL indicates the number of LNA-flanked amide bonds. DNA target (ON21)=TGTAACTGAGGTAAGAGG; RNA target (ON22)=UGUAACUGAGGUAAGAGG. Truncated DNA target (ON23)=AGGTAAGAGG. Truncated RNA target (ON24)=AGGUAAGAGG. ΔTₘ=modified−control. Bases in lower case italic remain single stranded on duplex formation and do not contribute to Tₘ. Representative melting curves are given (FIG. 15-18).

TABLE S4
Sequences of oligonucleotides used in crystallo-
graphic studies.

| ON | Sequence (5′ → 3′) | backbone |
|---|---|---|
| ON26^{xDNA} | CTTTTCTTTG | DNA |
| ON27^{xRNA} | CAAAGAAAAG | RNA |
| ON28^{xDNA-Am-} | CTT*TTCTTTG | DNA |
| ^{DNA} | | |
| ON29^{xLNA-Am-} | CTT*TTCTTTG | DNA |
| ^{DNA} | | |
| ON30^{xLNA-Am-} | CTT*TTCTTTG | DNA |
| ^{LNA} | | |

T indicates a locked sugar and * indicates an amide bond in place of phosphodiester.

T indicates a locked sugar and * indicates an amide bond in place of a phosphodiester.

TABLE S5
Summary of data processing for XRD structures of DNA:RNA hybrids containing amide and LNA
modifications. Data was validated using pdb validation. Each dataset was collected from
a single crystal, values shown in parenthesis are for the highest resolution shell.

| | | | | | ON29^{xLNA-Am-DNA}: | | ON30^{xLNA-Am-LNA}: | |
|---|---|---|---|---|---|---|---|---|
| Data collection | ON26^{xDNA}:ON27^{XRNA} | | ON28^{xDNA-Am-DNA}:ON27^{×RNA} | | ON27^{xRNA} | | ON27^{xRNA} | |
| PDB ID | 7NRP | | 7OOS | | 7OZZ | | 7OOO | |
| Space group | P 6₁ | | P 6₁ | | P 6₁ | | P 3₂21 | |
| Cell dimensions | | | | | | | | |
| a, b, c / Å | 53.34, 53.34, 45.88 | | 55.14, 55.14, 45.96 | | 54.12, 54.12, 48.84 | | 49.15, 49.15, 90.96 | |
| α, β, γ / ° | 90.00 90.00 | | 90.00 90.00 | | 90.00 90.00 | | 90.00 90.00 | |
| | 120.00 | | 120.00 | | 120.00 | | 120.00 | |
| Detector | 19.8 | | 48.5 | | 48.5 | | 17.0 | |
| distance / cm | | | | | | | | |
| Exposure time | 0.090 | | 0.038 | | 0.056 | | 0.040 | |
| per image / s | | | | | | | | |
| Resolution (Å) | 27.17-2.67 | | 47.75-2.60 | | 27.06-2.70 | | 31.08-2.56 | |
| | (2.80-2.67) | | (2.70-2.60) | | (2.77-2.70) | | (2.63-2.56) | |
| No. unique | 2243 | | 2517 | | 2283 | | 4398 | |
| reflections | | | | | | | | |
| Rₘₑₐₛ | 0.081 | (1.776) | 0.121 | (1.124) | 0.055 | (1.816) | 0.078 | (3.673) |
| I/σ(I) | 25.2 | (1.7) | 8.9 | (2.2) | 23.0 | (1.1) | 19.8 | (0.8) |
| CC_{1/2} | 0.996 | (0.646) | 0.943 | (0.645) | 0.999 | (0.421) | 0.999 | (0.329) |
| Completeness/% | 98.8 | (97.0) | 99.9 | (100.0) | 99.9 | (92.6) | 99.4 | (94.2) |
| Multiplicity | 19.3 | (16.0) | 7.5 | (7.1) | 9.7 | (9.0) | 18.7 | (18.5) |
| Refinement | | | | | | | | |
| Resolution / Å | 27.17-2.67 | | 47.75-2.60 | | 27.06-2.70 | | 31.08-2.56 | |
| | (2.76-2.67) | | (2.69-2.60) | | (2.79-2.70) | | (2.65-2.56) | |
| No. reflections | 2226 | | 2502 | | 2267 | | 4349 | |
| R_{work} / R_{free} | 0.170, 0.191 | | 0.218, 0.234 | | 0.200, 0.237 | | 0.237, 0.261 | |
| No. atoms / ASU | 422 | | 419 | | 416 | | 837 | |
| DNA | 197 | | 196 | | 198 | | 434 | |
| RNA | 217 | | 217 | | 217 | | 400 | |
| Mg²⁺ | — | | — | | — | | 2 | |
| As | 1 | | — | | — | | — | |
| Sr²⁺ | — | | 2 | | — | | — | |
| K⁺ | — | | — | | — | | — | |
| Ligand atoms | 7 | | 4 | | — | | 1 | |
| Wilson B factor | 75.0 | | 68.0 | | 102.1 | | 95.1 | |
| (Å²) | | | | | | | | |
| r.m.s. deviations | | | | | | | | |

| | | | | |
|---|---|---|---|---|
| Bond lengths / Å | 0.009 | 0.005 | 0.007 | 0.008 |
| Bond angles / Å | 1.20 | 0.53 | 0.76 | 0.94 |
| r.m.s.d Unmod / Å | — | 0.45 | 0.42 | 2.51 |

TABLE S6
DNA:RNA duplex crystallisation conditions.

| | Conditions | |
|---|---|---|
| Structure | Drop solution | Well solution |
| Unmodified | 0.5 mM duplex | MPD:H2O, 1:1 |
| ON26^{xDNA}:ON27^{xRNA} | 12 mM Mg(OAc)₂ | |
| | 0.6 mM spermidine•HCl | |
| | 0.075% β-octylglucoside | |
| | 12 mM sodium cacodylate pH 5.8 | |
| | 12% MPD | |
| Amide | 0.5 mM duplex | double the concentration |
| ON28^{xDNA-Am-DNA}:ON27^{xRNA} | 20 mM LiCl | of the drop solution without |
| | 40 mM SrCl₂•6H₂O | the oligonucleotide duplex |
| | 20 mM sodium cacodylate trihydrate | |
| | pH 7.0 | |
| | 15% (v/v) MPD | |
| | 6.0 mM spermine tetrahydrochloride | |
| LNA-amide | 0.5 mM duplex | double the concentration |
| ON29^{xLNA-Am-DNA}:ON27^{xRNA} | 40 mM NaCl | of the drop solution without |
| | 6.0 mM KCl | the oligonucleotide duplex |
| | 20 mM sodium cacodylate trihydrate | |
| | pH 7.0 | |
| | 17.5% MPD | |
| | 6.0 mM spermine tetrahydrochloride | |
| LNA-Am-LNA | 0.5 mM duplex | double the concentration |
| ON30^{xLNA-Am-LNA}:ON27^{xRNA} | 40 mM NaCl | of the drop solution without |
| | 6.0 mM KCl | the oligonucleotide duplex |
| | 20 mM sodium cacodylate trihydrate | |
| | pH 6.0 | |
| | 15% (v/v) MPD | |
| | 6.0 mM spermine tetrahydrochloride | |

While specific embodiments of the invention have been described for the purpose of reference and illustration, various modifications will be apparent to a person skilled in the art without departing from the scope of the invention as defined by the appended claims.

### REFERENCES

1. Smith, C. I. E. & Zain, R. Therapeutic Oligonucleotides: State of the Art. Annu. Rev. Pharmacol. Toxicol. 59, 605-630 (2019).
2. Khvorova, A. & Watts, J. K. The chemical evolution of oligonucleotide therapies of clinical utility. Nat. Biotechnol. 35, 238-248 (2017).
3. Rinaldi, C. & Wood, M. J. A. Antisense oligonucleotides: the next frontier for treatment of neurological disorders. Nat. Revi. Neurol. 14, 9-21 (2018).
4. Liang, X.-H., Sun, H., Nichols, J. G. & Crooke, S. T. RNase H1-Dependent Antisense Oligonucleotides Are Robustly Active in Directing RNA Cleavage in Both the Cytoplasm and the Nucleus. Mol. Ther. 25, 2075-2092 (2017).
5. Dominski, Z. & Kole, R. Restoration of correct splicing in thalassemic pre-mRNA by antisense oligonucleotides. Proc. Natl. Acad. Sci. U.S.A. 90, 8673-8677 (1993).
6. Sierakowska, H., Sambade, M. J., Agrawal, S. & Kole, R. Repair of thalassemic human beta-globin mRNA in mammalian cells by antisense oligonucleotides. Proc. Natl. Acad. Sci. U.S.A. 93, 12840-12844 (1996).
7. Aartsma-Rus, A. et al. Therapeutic antisense-induced exon skipping in cultured muscle cells from six different DMD patients. Hum. Mol. Genet. 12, 907-914 (2003).
8. Setten, R. L., Rossi, J. J. & Han, S.-p. The current state and future directions of RNAi-based therapeutics. Nat. Rev. Drug Discov. 18, 421-446 (2019).
9. Warner, K. D., Hajdin, C. E. & Weeks, K. M. Principles for targeting RNA with drug-like small molecules. Nat. Rev. Drug Discov. 17, 547-558 (2018).
10. Freier, S. M. & Altmann, K.-H. The ups and downs of nucleic acid duplex stability: Structure-stability studies on chemically-modified DNA:RNA duplexes. Nucleic Acids Res. 25, 4429-4443 (1997).
11. Cummins, L. L. et al. Characterization of fully 2′-modified oligoribonucleotide hetero- and homoduplex hybridization and nuclease sensitivity. Nucleic Acids Res. 23, 2019-2024 (1995).
12. Monia, B. P., Johnston, J. F., Sasmor, H. & Cummins, L. L. Nuclease resistance and antisense activity of modified oligonucleotides targeted to Ha-ras. J. Biol. Chem. 271, 14533-14540 (1996).
13. Martin, P. A New Access to 2′-O-Alkylated Ribonucleosides and Properties of 2′-O-Alkylated Oligoribonucleotides. Helv. Chim. Acta 78, 486-504 (1995).
14. Eckstein, F. Phosphorothioates, essential components of therapeutic oligonucleotides. Nucleic Acid Ther. 24, 374-387 (2014).
15. Xiong, H., Veedu, R. N. & Diermeier, S. D. Recent Advances in Oligonucleotide Therapeutics in Oncology. Int. J. Mol. Sci. 22, 3295 (2021).
16. Roberts, T. C., Langer, R. & Wood, M. J. A. Advances in oligonucleotide drug delivery. Nat. Rev. Drug Discov. 19, 673-694 (2020).
17. Godfrey, C. et al. Delivery is key: lessons learnt from developing splice-switching antisense therapies. EMBO Mol. Med. 9, 545-557 (2017).
18. Meade, B. R. et al. Efficient delivery of RNAi prodrugs containing reversible charge-neutralizing phosphotriester backbone modifications. Nat. Biotechnol. 32, 1256-1261 (2014).
19. Nielsen, P., Egholm, M., Berg, R. & Buchardt, O. Sequence-selective recognition of DNA by strand displacement with a thymine-substituted polyamide. Science 254, 1497-1500 (1991).
20. Nielsen, P. E., Egholm, M. & Buchardt, O. Peptide nucleic acid (PNA). A DNA mimic with a peptide backbone. Bioconjugate Chem. 5, 3-7 (1994).
21. Koppelhus, U. & Nielsen, P. E. Cellular delivery of peptide nucleic acid (PNA). Adv. Drug. Deliv. Rev. 55, 267-280 (2003).
22. Saarbach, J., Sabale, P. M. & Winssinger, N. Peptide nucleic acid (PNA) and its applications in chemical biology, diagnostics, and therapeutics. Curr. Opin. Chem. Biol. 52, 112-124 (2019).
23. Idziak, I., Just, G., Damha, M. J. & Giannaris, P. A. Synthesis and hybridization properties of amide-linked thymidine dimers incorporated into oligodeoxynucleotides. Tetrahedron Lett. 34, 5417-5420 (1993).
24. Lebreton, J., Waldner, A., Lesueur, C. & De Mesmaeker, A. Antisense Oligonucleotides with Alternating Phosphodiester-“Amide-3” Linkages. Synlett 1994, 137-140 (1994).
25. De Mesmaeker, A. et al. Amides as a New Type of Backbone Modification in Oligonucleotides. Angew. Chem. Int. Ed. 33, 226-229 (1994).
26. Hardcastle, T. et al. A Single Amide Linkage in the Passenger Strand Suppresses Its Activity and Enhances Guide Strand Targeting of siRNAs. ACS Chem. Biol. 13, 533-536 (2018).
27. Mutisya, D. et al. Amide linkages mimic phosphates in RNA interactions with proteins and are well tolerated in the guide strand of short interfering RNAs. Nucleic Acids Res. 45, 8142-8155 (2017).
28. Selvam, C., Thomas, S., Abbott, J., Kennedy, S. D. & Rozners, E. Amides as Excellent Mimics of Phosphate Linkages in RNA. Angew. Chem. Int. Ed. 50, 2068-2070 (2011).
29. Kotikam, V. & Rozners, E. Amide-Modified RNA: Using Protein Backbone to Modulate Function of Short Interfering RNAs. Acc. Chem. Res. 53, 1782-1790 (2020).
30. Koshkin, A. A. et al. LNA (Locked Nucleic Acids): Synthesis of the adenine, cytosine, guanine, 5-methylcytosine, thymine and uracil bicyclonucleoside monomers, oligomerisation, and unprecedented nucleic acid recognition. Tetrahedron 54, 3607-3630 (1998).
31. Obika, S. et al. Synthesis of 2′-O,4′-C-methyleneuridine and -cytidine. Novel bicyclic nucleosides having a fixed C3, -endo sugar puckering. Tetrahedron Lett. 38, 8735-8738 (1997).
32. Hagedorn, P. H. et al. Locked nucleic acid: modality, diversity, and drug discovery. Drug Discov. Today 23, 101-114 (2018).
33. Singh, S. K., A. Koshkin, A., Wengel, J. & Nielsen, P. LNA (locked nucleic acids): synthesis and high-affinity nucleic acid recognition. Chem. Commun., 455-456 (1998).
34. Singh, S. K. & Wengel, J. Universality of LNA-mediated high-affinity nucleic acid recognition. Chem. Commun., 1247-1248 (1998).
35. Bondensgaard, K. et al. Structural Studies of LNA:RNA Duplexes by NMR: Conformations and Implications for RNase H Activity. Chem. Eur. J. 6, 2687-2695 (2000).
36. Sharma, V. K. et al. Synthesis and biological properties of triazole-linked locked nucleic acid. Chem. Commun. 53, 8906-8909 (2017).
37. Kumar, P., Truong, L., Baker, Y. R., EI-Sagheer, A. H. & Brown, T. Synthesis, Affinity for Complementary RNA and DNA, and Enzymatic Stability of Triazole-Linked Locked Nucleic Acids (t-LNAs). ACS Omega 3, 6976-6987 (2018).
38. Kumar, P., EI-Sagheer, A. H., Truong, L. & Brown, T. Locked nucleic acid (LNA) enhances binding affinity of triazole-linked DNA towards RNA. Chem. Commun. 53, 8910-8913 (2017).
39. Thorpe, C., Epple, S., Woods, B., EI-Sagheer, A. H. & Brown, T. Synthesis and biophysical properties of carbamate-locked nucleic acid (LNA) oligonucleotides with potential antisense applications. Org. Biomol. Chem. 17, 5341-5348 (2019).
40. Lauritsen, A. & Wengel, J. Oligodeoxynucleotides containing amide-linked LNA-type dinucleotides: synthesis and high-affinity nucleic acid hybridization. Chem. Commun., 530-531 (2002).
41. Seth, P. P. & Swayze, E. E. Oligomeric compounds comprising neutrally linked terminal bicyclic nucleosides. WO/2009/124238 (2009).
42. Shivalingam, A., Tyburn, A. E. S., EI-Sagheer, A. H. & Brown, T. Molecular Requirements of High-Fidelity Replication-Competent DNA Backbones for Orthogonal Chemical Ligation. J. Am. Chem. Soc. 139, 1575-1583 (2017).
43. Koshkin, A. A., Fensholdt, J., Pfundheller, H. M. & Lomholt, C. A Simplified and Efficient Route to 2′-O, 4′-C-Methylene-Linked Bicyclic Ribonucleosides (Locked Nucleic Acid). J. Org. Chem. 66, 8504-8512 (2001).
44. Lebreton, J., Waldner, A., Fritsch, V., Wolf, R. M. & De Mesmaeker, A. Comparison of two amides as backbone replacement of the phosphodiester linkage in oligodeoxynucleotides. Tetrahedron Lett. 35, 5225-5228 (1994).
45. Peterson, M. A. et al. Amide-Linked Ribonucleoside Dimers Derived from 5′-Amino-5′-deoxy- and 3′-(Carboxymethyl)-3′-deoxynucleoside Precursors(1). J. Org. Chem. 64, 8183-8192 (1999).
46. Kotikam, V. & Rozners, E. Concurrent Hydrogenation of Three Functional Groups Enables Synthesis of C3′-Homologated Nucleoside Amino Acids. Org. Lett. 19, 4122-4125 (2017).
47. Albrecht, H., Jones, G. & Moffatt, J. 3′-Deoxy-3′-(dihydroxyphosphinylmethyl)nucleosides. Isophosphonate Analogs of Nucleoside 3′-Phosphates. J. Am. Chem. Soc. 92, 5511-5513 (1970).
48. Arzel, L. et al. Synthesis of Ribonucleosidic Dimers with an Amide Linkage from d-Xylose. J. Org. Chem. 81, 10742-10758 (2016).
49. Vorbrüggen, H. Some recent trends and progress in nucleoside synthesis. Acta Biochim. Pol. 43, 25-36 (1996).
50. Obika, S., Nakagawa, O., Hiroto, A., Hari, Y. & Imanishi, T. Synthesis and properties of a novel bridged nucleic acid with a P3′→N5′ phosphoramidate linkage, 5′-amino-2′,4′-BNA. Chem. Commun., 2202-2203 (2003).
51. Stork, G., Zhang, C., Gryaznov, S. & Schultz, R. Modified ollgonucleotides. Effect of 4 vs 5-atom chimeric internucleoside linkages on duplex stability. Tetrahedron Lett. 36, 6387-6390 (1995).
52. Ammenn, J., Altmann, K.-H. & Belluš, D. Aza-Claisen Rearrangement: Synthesis of 5′-branched 5′-aminothymidines. Helv. Chim. Acta 80, 1589-1606 (1997).
53. Brandsen, B. M., Hesser, A. R., Castner, M. A., Chandra, M. & Silverman, S. K. DNA-Catalyzed Hydrolysis of Esters and Aromatic Amides. J. Am. Chem. Soc. 135, 16014-16017 (2013).
54. Kopka, M. L., Lavelle, L., Han, G. W., Ng, H.-L. & Dickerson, R. E. An Unusual Sugar Conformation in the Structure of an RNA/DNA Decamer of the Polypurine Tract May Affect Recognition by RNase H. J. Mol. Biol. 334, 653-665 (2003).
55. Kang, S.-H., Cho, M.-J. & Kole, R. Up-Regulation of Luciferase Gene Expression with Antisense Oligonucleotides: Implications and Applications in Functional Assay Development. Biochemistry 37, 6235-6239 (1998).
56. Epple, S., Thorpe, C., Baker, Y. R., EI-Sagheer, A. H. & Brown, T. Consecutive 5′- and 3′-amide linkages stabilise antisense oligonucleotides and elicit an efficient RNase H response. Chem. Commun. 56, 5496-5499 (2020).
57. Bailey, J. K., Shen, W., Liang, X.-h. & Crooke, S. T. Nucleic acid binding proteins affect the subcellular distribution of phosphorothioate antisense oligonucleotides. Nucleic Acids Res. 45, 10649-10671 (2017).
58. Rigo, F. et al. Synthetic oligonucleotides recruit ILF2/3 to RNA transcripts to modulate splicing. Nat. Chem. Biol. 8, 555-561 (2012).

### REFERENCES FOR SUPPLEMENTARY INFORMATION

1. Thorpe, C., Epple, S., Woods, B., EI-Sagheer, A. H. & Brown, T. Synthesis and biophysical properties of carbamate-locked nucleic acid (LNA) oligonucleotides with potential antisense applications. Org. Biomol. Chem. 17, 5341-5348 (2019).
2. Koshkin, A. A., Fensholdt, J., Pfundheller, H. M. & Lomholt, C. A Simplified and Efficient Route to 2′-O, 4′-C-Methylene-Linked Bicyclic Ribonucleosides (Locked Nucleic Acid). J. Org. Chem. 66, 8504-8512 (2001).
3. Obika, S., Nakagawa, O., Hiroto, A., Hari, Y. & Imanishi, T. Synthesis and properties of a novel bridged nucleic acid with a P3′→N5′ phosphoramidate linkage, 5′-amino-2′,4′-BNA. Chem. Commun., 2202-2203 (2003).
4. Kopka, M. L., Lavelle, L., Han, G. W., Ng, H.-L. & Dickerson, R. E. An Unusual Sugar Conformation in the Structure of an RNA/DNA Decamer of the Polypurine Tract May Affect Recognition by RNase H. J. Mol. Biol. 334, 653-665 (2003).
5. Winter, G. & McAuley, K. E. Automated data collection for macromolecular crystallography. Methods 55, 81-93 (2011).
6. Winter, G. et al. DIALS: implementation and evaluation of a new integration package. Acta Crystallogr. D Struct. Biol. 74, 85-97 (2018).
7. Winter, G. xia2: An Expert System for Macromolecular Crystallography Data Reduction. J. Appl. Crystallogr. 43, 186-190 (2010).
8. Lebedev, A. A. et al. JLigand: a graphical tool for the CCP4 template-restraint library. Acta Crystallogr. D Biol. Crystallogr. 68, 431-440 (2012).
9. Rossmann, M. G. The molecular replacement method. Acta Crystallogr. A 46 (Pt 2), 73-82 (1990).
10. McCoy, A. J. et al. Phaser crystallographic software. J. Appl. Crystallogr. 40, 658-674 (2007).
11. Murshudov, G. N., Vagin, A. A. & Dodson, E. J. Refinement of macromolecular structures by the maximum-likelihood method. Acta Crystallogr. D Biol. Crystallogr. 53, 240-255 (1997).
12. Murshudov, G. N. et al. REFMAC5 for the refinement of macromolecular crystal structures. Acta Crystallogr. D Biol. Crystallogr. 67, 355-367 (2011).
13. Liebschner, D. et al. Macromolecular structure determination using X-rays, neutrons and electrons: recent developments in Phenix. Acta Crystallogr. D Struct. Biol. 75, 861-877 (2019).
14. Long, F. et al. AceDRG: a stereochemical description generator for ligands. Acta Crystallogr. D Struct. Biol. 73, 112-122 (2017).
15. Winn, M. D. et al. Overview of the CCP4 suite and current developments. Acta Crystallogr. D Biol. Crystallogr. 67, 235-242 (2011).
16. Evans, P. R. An introduction to data reduction: space-group determination, scaling and intensity statistics. Acta Crystallogr. D Biol. Crystallogr. 67, 282-292 (2011).
17. Kang, S.-H., Cho, M.-J. & Kole, R. Up-Regulation of Luciferase Gene Expression with Antisense Oligonucleotides: Implications and Applications in Functional Assay Development. Biochemistry 37, 6235-6239 (1998).
18. Palatinus, L. & Chapuis, G. SUPERFLIP—a computer program for the solution of crystal structures by charge flipping in arbitrary dimensions. J. Appl. Crystallogr. 40, 786-790 (2007).
19. Parois, P., Cooper, R. I. & Thompson, A. L. Crystal structures of increasingly large molecules: meeting the challenges with CRYSTALS software. Chem. Cent. J. 9, 30 (2015).
20. Cooper, R. I., Thompson, A. L. & Watkin, D. J. CRYSTALS enhancements: dealing with hydrogen atoms in refinement. J. Appl. Crystallogr. 43, 1100-1107 (2010).

1. An oligonucleotide having a 5′ and a 3′ end and comprising a sequence of nucleosides linked together by inter-nucleoside linkages, wherein:
at least one inter-nucleoside linkage is an amide linker moiety;
at least one inter-nucleoside linkage is a phosphorothioate linker moiety; and
at least one nucleoside present in the oligonucleotide is a locked nucleoside;
wherein the at least one locked nucleoside is directly attached to the 3′ end or the 5′ end of the amide linker moeity;
or a pharmaceutically acceptable salt or solvate thereof.

2. The oligonucleotide according to claim 1, or a pharmaceutically acceptable salt or solvate thereof, wherein the amide linker has the structure shown below; 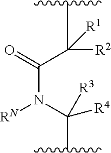
wherein:
R¹ and R² are each independently selected from hydrogen, (1-2C)alkyl, hydroxy, amino or halo;
R³ and R⁴ are each independently selected from hydrogen, (1-2C)alkyl, hydroxy, amino or halo; and
R^{N} is selected from hydrogen or (1-2C)alkyl.

3. The oligonucleotide according to claim 1 or claim 2, or a pharmaceutically acceptable salt or solvate thereof, wherein the locked nucleoside has the general structure shown below: 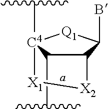
wherein:
Q₁ is selected from CR^{p}R^{q}, O, S or NR^{a}, wherein R^{p} and R^{q} are each independently selected from H, (1-4C)alkyl or halo, and R^{a} is selected from hydrogen or (1-4C)alkyl;
B′ is a nucleobase or nucleobase analogue; and
either
a) one of X₁ and X₂ is (CR^{a}R^{b})ₓ (where x is selected from 1 or 2) and the other is selected from CR^{a1}R^{b1}, O, NR^{c} or S;
wherein each of R^{a}, R^{b}, R^{a1} and R^{b1} are independently selected from hydrogen, (1-2C)alkyl, hydroxy, amino, halo or mercapto; and R^{c} is selected from hydrogen or a (1-6C)alkyl; or
b) one of X₁ and X₂ is O and the other is NR^{c}.

4. An oligonucleotide according to any of claims 1 to 3, or a pharmaceutically acceptable salt or solvate thereof, wherein the oligonucleotide comprises a moiety of the formula (I) below: 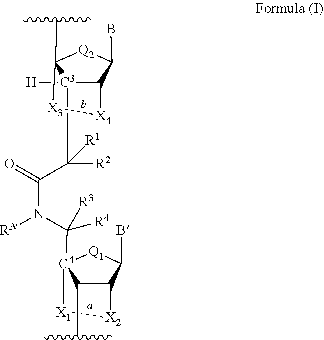
wherein:
C³ is a 3′ carbon;
C⁴ is a 4′ carbon;
Q₁ is selected from CR^{p}R^{q}, O, S or NR^{a}, wherein R^{p} and R^{q} are each independently selected from H, (1-4C)alkyl or halo and R^{a} is selected from hydrogen or (1-4C)alkyl;
Q₂ is selected from CR^{p}R^{q}, O, S or NR^{a}, wherein R^{p} and R^{q} are each independently selected from H, (1-4C)alkyl or halo and R^{a} is selected from hydrogen or (1-4C)alkyl;
B and B′ are each independently a nucleobase;
either both of bonds a and b are present, or only one of bonds a and b is present;
either:
a) if bond a is present, one of X₁ and X₂ is (CR^{a}R^{b})ₓ (where x is selected from 1 or 2) and the other is selected from CR^{a1}R^{b1}, O, NR^{c} or S; or
b) if bond a is present, one of X₁ and X₂ is O and the other is NR^{c}; or
c) if bond a is absent, one of X₁ and X₂ is H and the other is selected from H, C₁₋₄alkoxy, F, OH, OR^{c}, O(CH₂)ₙOR^{c} (where n is selected from 1, 2 or 3) or NH₂;
wherein each of R^{a}, R^{b}, R^{a1} and R^{b1} are independently selected from hydrogen, (1-2C)alkyl, hydroxy, amino, halo or mercapto; and
R^{c} is selected from hydrogen or a (1-6C)alkyl;
either:
a) if bond b is present, one of X₃ and X₄ is (CR^{d}R^{e})_{y} (wherein y is selected from 1 or 2) and the other is selected from CR^{d1}R^{e1}, O, NR^{f} or S; or
b) if bond b is present, one of X₃ and X₄ is O and the other is NR^{f};
c) if bond b is absent, one of X₃ and X₄ is H and the other is selected from H, C₁₋₄alkoxy, F, OH, OR^{f}, O(CH₂)ₘOR^{f} (where m is selected from 1, 2 or 3) or NH₂;
wherein R^{d} and R^{e} are independently selected from hydrogen, (1-2C)alkyl, hydroxy, amino, halo or mercapto; and
R^{f} is selected from hydrogen or a (1-6C)alkyl;
R¹ and R² are each independently selected from hydrogen, (1-2C)alkyl, hydroxy, amino or halo;
R³ and R⁴ are each independently selected from hydrogen, (1-2C)alkyl, hydroxy, amino or halo; and
R^{N} is selected from hydrogen or (1-2C)alkyl.

5. An oligonucleotide according to claim 4, or a pharmaceutically acceptable salt or solvate thereof, wherein the at least one phosphorothioate linker is:
directly attached to the 3′ end of the dinucleotide moiety according to formula (I); and/or
directly attached to the 5′ end of the dinucleotide moiety according to formula (I).

6. An oligonucleotide according to any one of claim 4 or 5, or a pharmaceutically acceptable salt or solvate thereof, wherein Q₁ is selected from CH₂, CF₂, O or S.

7. An oligonucleotide according to any of claims 4 to 6, or a pharmaceutically acceptable salt or solvate thereof, wherein Q₁ is O.

8. An oligonucleotide according to any one of claims 4 to 7, or a pharmaceutically acceptable salt or solvate thereof, wherein:
if bond a is present, then one of X₁ and X₂ is CR^{a}R^{b} and the other is selected from CR^{a1}R^{b1}, O, NR^{c} or S; or
if bond a is absent, one of X₁ and X₂ is H and the other is selected from H, C₁₋₄alkoxy, F, OH, OR^{c}, O(CH₂)ₙOR^{c} (where n is selected from 1, 2 or 3) or NH₂;
wherein each of R^{a}, R^{b}, R^{a1} and R^{b1} are independently selected from hydrogen, (1-2C)alkyl, hydroxy, amino or halo; and
R^{c} is selected from hydrogen or (1-6C)alkyl.

9. An oligonucleotide according to any of claims 4 to 8, wherein:
if bond a is present, X₁ is CR^{a}R^{b} and X₂ is selected from O, NR^{c} or S; or
if bond a is absent, X₁ is H and X₂ is selected from H, methoxy, F, OH, O(CH₂)₂OMe;
wherein R^{a} and R^{b} are independently selected from hydrogen or methyl, and
R^{c} is selected from hydrogen or methyl.

10. An oligonucleotide according to any of claims 4 to 9, or a pharmaceutically acceptable salt or solvate thereof, wherein:
a) if bond a is present, X₁ is CH₂ and X₂ is O; or
b) if bond a is absent, X₁ is H and X₂ is H or OH;

11. An oligonucleotide according to any of claims 4 to 10, wherein Q₂ is selected from CH₂, CF₂, O or S.

12. An oligonucleotide according to claim 11, or a pharmaceutically acceptable salt or solvate thereof, wherein Q₂ is O.

13. An oligonucleotide according to any of claims 4 to 12, or a pharmaceutically acceptable salt or solvate thereof, wherein:
if bond b is present, then one of X₃ and X₄ is CR^{d}R^{e} and the other is selected from CR^{d1}R^{e1} O, NR^{f} or S; or
if bond b is absent, one of X₃ and X₄ is H and the other is selected from H, C₁₋₄alkoxy, F, OH, OR^{f}, O(CH₂)ₘOR^{f} or NH₂;
wherein m is selected from 1, 2 or 3,
wherein each of R^{d}, R^{e}, R^{d1} and R^{e1} are independently selected from hydrogen, (1-2C)alkyl, hydroxy, amino or halo; and
R^{f} is selected from hydrogen or (1-6C)alkyl.

14. An oligonucleotide according to any of claims 4 to 13, or a pharmaceutically acceptable salt or solvate thereof, wherein:
if bond b is present, X₃ is CR^{d}R^{e} and X₄ is selected from O, NR^{c} or S; or
if bond b is absent, X₃ is H and X₄ is selected from H, methoxy, F, OH, O(CH₂)₂OMe;
wherein:
R^{d} and R^{e} are independently selected from hydrogen or methyl, and
R^{f} is selected from hydrogen or methyl.

15. An oligonucleotide according to any of claims 4 to 14, or a pharmaceutically acceptable salt or solvate thereof, wherein:
if bond b is present, X₃ is CH₂ and X₄ is O; or
if bond b is absent, X₃ is H and X₄ is H or OH.

16. An oligonucleotide according to any of claims 4 to 15, or a pharmaceutically acceptable salt or solvate thereof, wherein R¹, R², R³, R⁴ and R^{N} are each independently selected from hydrogen or methyl.

17. An oligonucleotide according to any of claims 4 to 16, or a pharmaceutically acceptable salt or solvate thereof, wherein both of bonds a and b are present, thus the oligonucleotide comprises a moiety of Formula (Ia) below: 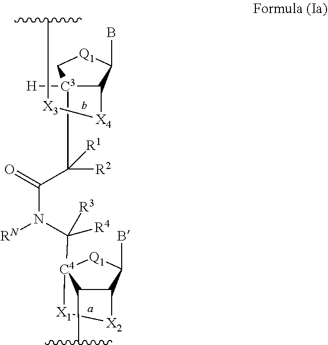
wherein C³, C⁴, Q₁, Q₂, B, B′, X₁, X₂, X₃, X₄, R¹, R², R³, R⁴ and R^{N} are as defined in any one of the preceding claims.

18. An oligonucleotide according to any one of claims 1 to 17, or a pharmaceutically acceptable salt or solvate thereof, wherein the oligonucleotide comprises a moiety of formula (IIa) or (IIb) below: 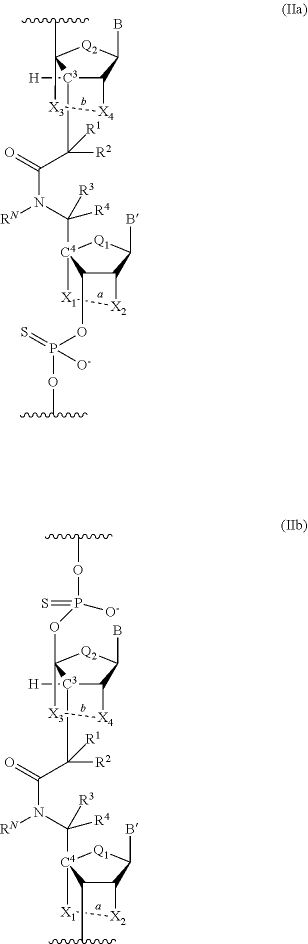
wherein C³, C⁴, Q₁, Q₂, B, B′, X₁, X₂, X₃, X₄, R^{1a}, R^{1b}, R^{2a}, R^{2b} and R^{N} are as defined in any one of claims 2 to 15.

19. An oligonucleotide according to claim 18, or a pharmaceutically acceptable salt or solvate thereof, wherein the oligonucleotide comprises a moiety of the structure (IIc) or (IId) below: 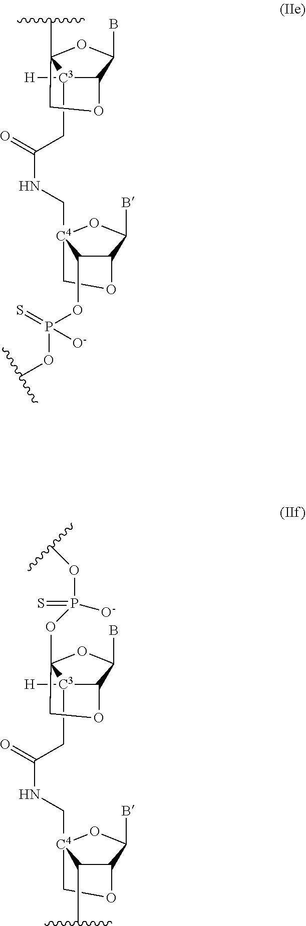
wherein B and B′ are as defined in any one of the preceding claims.

20. An oligonucleotide according to any one of the preceding claims, or a pharmaceutically acceptable salt or solvate thereof, wherein the oligonucleotide comprises a moiety of the structure (IIIa) below: 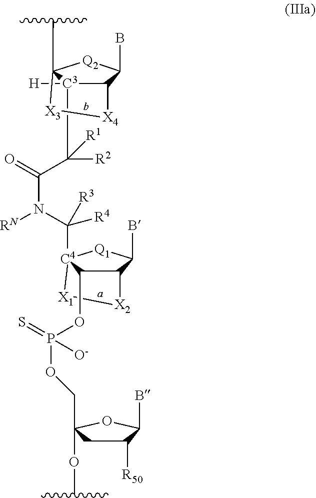
C³, C⁴, Q₁, Q₂, B, B′, X₁, X₂, X₃, X₄, R¹, R², R³, R⁴ and R^{N} are as defined in any one of the preceding claims;
wherein B″ is independently a nucleobase; and
R₅₀ is selected from H, C₁₋₄alkoxy, F, OH, OR⁹, O(CH₂)ₚOR⁹ or NH₂, wherein p is selected from 1, 2 or 3 and R⁹ is selected from hydrogen or a (1-6C)alkyl.

21. An oligonucleotide according to any one of the preceding claims, or a pharmaceutically acceptable salt or solvate thereof, for use in therapy.

22. An oligonucleotide according to any one of the preceding claims, or a pharmaceutically acceptable salt or solvate thereof, for use in the treatment of a viral infection, cancer, a genetic disorder, a metabolic disease or a bacterial infection.
